(19) United States
(12) Patent Application Publication
(10) Pub. No.: US 2023/0014181 A1
(43) Pub. Date: Jan. 19, 2023

(54) CULTURE SYSTEM AND METHODS FOR IMPROVED MODELING OF NEUROLOGICAL CONDITIONS

(71) Applicant: Genentech, Inc., South San Francisco, CA (US)

(72) Inventors: Shirley Sum Yi NG PALACE, Brisbane, CA (US); Benny CHIH, Millbrae, CA (US); Reina Angelica Salam BASSIL, Lancaster, CA (US); Kenneth McCarter SHIELDS, Gig Harbor, WA (US)

(21) Appl. No.: 17/842,715

(22) Filed: Jun. 16, 2022

Related U.S. Application Data
(60) Provisional application No. 63/212,063, filed on Jun. 17, 2021.

(51) Int. Cl. C12M 1/36; C12N 5/0793; C12M 1/32; C12M 1/00; G01N 33/50

(52) U.S. Cl. CPC C12M 41/48; C12N 5/0619; C12M 23/12; C12M 29/26; G01N 33/5058; C12N 2506/45

(57) ABSTRACT

The present application provides a pluripotent stem cell-derived neuronal culture system for use in modeling neurodegenerative diseases, drug screening and target discovery; and methods of generating homogenous, terminally differentiated neuronal culture from pluripotent stem cells, and compositions resulting thereof; as well as automated cell culture systems that sustain long-term differentiation, maturation and/or growth of neuronal cells for use in modeling neurodegenerative diseases.

### CROSS REFERENCE TO RELATED APPLICATIONS

This application claims the priority benefit of U.S. Provisional Application No. 63/212,063, filed Jun. 17, 2021, which is incorporated herein by reference in its entirety.

### FIELD OF THE INVENTION

The present disclosure relates generally to automated culture systems, methods of using the automated culture systems to generate homogenous populations of fully differentiated progeny cells and neurological disease models, as well as improved systems for modeling neurological conditions and diseases.

### BACKGROUND OF THE INVENTION

Current rodent Alzheimer's disease (AD) models recapitulate amyloid plaque-associated pathologies, however, amyloid-mediated tau pathologies and neuronal loss have not been robustly modeled, precluding the study of Aβ-induced Tau pathological events and translation to human patients. Developing of preclinical models that could robustly mimic AD pathophysiology is needed for translational drug development. Advancement in human induced pluripotent stem cell (iPSC) neuron and microglial differentiation protocols have created new possibilities for preclinical human disease modeling using physiologically relevant cells, and can be combined with powerful genetic and molecular tools to discover new targets and drug screening. However, iPSC differentiation and culturing protocols are long and variable, posing challenges to maintaining consistency. In addition, although many iPSC models have been generated, robust amyloid plaque formation, phosphorylated Tau, or neuronal loss phenotypes have not been observed. Here, we generated an automated, consistent, and long-term culturing platform of human iPSC neurons, astrocytes, and microglia for high throughput, high content imaging, and disease modeling. Using this platform, we generated a human iPSC AD model, which manifested multiple key human AD pathological hallmarks including amyloid-O (AD) plaques, dystrophic neurites around plaques, synapse loss, dendrite retraction, axon fragmentation, phospho-Tau induction, and neuronal cell death in one model. Using this model, we showed human iPSC microglia internalized and compacted Aβ to generate and surround the plaques, thereby conferring some neuroprotection. This protection was lost in a neuroinflammatory culture condition even though plaque formation increased. Anti-Aβ antibodies protected neurons from these pathologies and were most effective prior to pTau induction. We conducted a focused screen and identified several known kinases in AD signaling pathways such as GSK3, DLK, Fyn, indicating that pathological signaling events are preserved in this system. Taken together, these results demonstrate that this model can be used for target discovery and drug development.

### BRIEF SUMMARY OF THE INVENTION

In some aspects, the disclosure provides an automated cell culture system for facilitating neuronal differentiation and/or promoting long-term neuronal growth, wherein the automated cell culture system comprises one or more rounds of automated culture media replacements; and wherein the automated cell culture system sustains differentiation, maturation and/or growth of neuronal cells for at least about any one of: 30, 60, 80, 90, 120, or 150 days. In some embodiments, the automated culture media replacement comprises automated culture media aspiration and automated culture media replenishment; and/or the cell culture system comprises one or more 96-well plates; or one or more 384-well plates. In some embodiments, the automated culture media aspiration comprises aspiration with a pipet tip, wherein: the distal end of the pipet tip is at about 1 mm above the bottom surface of the well before, during and/or after the aspiration. In some embodiments, the automated culture media aspiration comprises aspiration with a pipet tip, wherein: the pipet tip is at an angle of about 90° to the bottom surface of the well before, during and/or after the aspiration. In some embodiments, the automated culture media aspiration comprises aspiration with a pipet tip, wherein: the pipet tip has a displacement of no more than 0.1 mm from the center of the well before, during and/or after the aspiration; optionally wherein the pipet tip is at the center of the well before, during and/or after the aspiration (no displacement). In some embodiments, the automated culture media aspiration comprises aspiration with a pipet tip, wherein: (a) the speed of media aspiration is no more than about 7.5 μl/s; and/or (b) the start of media aspiration is about 200 ms subsequent to the pipet tip being placed 1 mm above the bottom surface of the well. In some embodiments, the automated culture media aspiration comprises aspiration with a pipet tip, wherein: (a) the pipet tip is inserted into the well at a speed of about 5 mm/s prior to aspiration; and/or (b) the pipet tip is withdrawn from the well at a speed of about 5 mm/s after the aspiration. In some embodiments, the cell culture system comprises a 384-well plate; further wherein the automated cell culture system comprises automated discarding of a used rack of 384-pipet tips and automated engagement of a new rack of 384-pipet tips subsequent to each round of media aspiration. In some embodiments, the cell culture system comprises one or more batches of 384-well plates, wherein each batch comprises up to twenty-five 384-well plates arranged in 5 columns and 5 rows; further wherein: the automated cell culture system comprises automated discarding of up to 25 corresponding used racks of 384-pipet tips and automated engagement of up to 25 corresponding new racks of 384-pipet tips subsequent to each round of media aspiration.

In some embodiments according to any one of the cell culture systems described herein, the automated culture media replenishment comprises dispensing media with a pipet tip, wherein: (a) the distal end of the pipet tip is at about 1 mm above the bottom surface of the well before the dispensing; and/or (b) the pipet tip is withdrawn from the well at a speed of about 1 mm/s during the dispensing. In some embodiments, the automated culture media replenishment comprises dispensing media with a pipet tip, wherein: the pipet tip is at an angle of about 90° to the bottom surface of the well before and/or during the dispensing. In some embodiments, the automated culture media replenishment comprises dispensing media with a pipet tip, wherein: the pipet tip has a displacement of no more than 0.1 mm from the center of the well before, and/or during the dispensing, optionally wherein the pipet tip is at the center of the well before, and/or during the dispensing (no displacement). In some embodiments, the cell culture system comprises a 384-well tissue plate; wherein the automated culture media replenishment comprises dispensing media with a pipet tip, wherein: (a) the pipet tip is displaced to contact a first side of the well 1 mm from the center in a first direction, at a height of about 12.40 mm above the bottom of the well at a speed of about 100 mm/s; and/or (b) the pipet tip is displaced to contact a second side of the well 1 mm from the center in a second direction, at a height of about 12.40 mm above the bottom of the well at a speed of about 100 mm/s, optionally wherein the first direction is at an angle of about 1800 to the second direction. In some embodiments, the automated culture media replenishment comprises dispensing media with a pipet tip, wherein: (a) the speed of media dispensing is no more than about 1.5 μl/s; (b) the acceleration of media dispensing is about 500 μl/s²; (c) the deceleration of media dispensing is about 500 μl/s²; and/or (d) the start of media dispensing is about 200 ms subsequent to the pipet tip being placed 1 mm above the bottom surface of the well. In some embodiments, the automated culture media replenishment comprises dispensing media with a pipet tip, wherein: (a) the pipet tip is inserted into the well at a speed of about 5 mm/s prior to dispensing; and/or (b) the pipet tip is withdrawn from the well at a speed of about 5 mm/s after the dispensing. In some embodiments, the cell culture system comprises a 384-well plate; further wherein the automated cell culture system comprises automated discarding of a used rack of 384-pipet tips and automated engagement of a new rack of 384-pipet tips subsequent to each round of media dispensing. In some embodiments, the cell culture system comprises one or more batches of 384-well plates, wherein each batch comprises up to twenty-five 384-well plates arranged in 5 columns and 5 rows; further wherein the automated cell culture system comprises automated discarding of up to 25 corresponding used racks of 384-pipet tips and automated engagement of up to 25 corresponding new racks of 384-pipet tips subsequent to each round of media dispensing.

In some embodiments according to any one of the cell culture systems described herein, the time interval between two rounds of culture media replacements is about any one of: 1, 2, 3, 4, 5, 6, 7, 8, 9, or 10 days. In some embodiments, the time interval between two rounds of culture media replacements is about 3 or 4 days. In some embodiments, about any one of: 30%, 40%, 50%, 60%, 70%, or 80% of culture media is replaced in one or more rounds of culture media replacement. In some embodiments, about any one of: 30%, 40%, 50%, 60%, 70%, or 80% of culture media is replaced in each round of culture media replacement. In some embodiments, about 50% of culture media is replaced in one or more rounds of culture media replacement. In some embodiments, about 50% of culture media is replaced in each round of culture media replacement.

In some aspects, the disclosure provides a method of generating homogenous and terminally differentiated neurons from pluripotent stem cells, comprising: (a) generating a pluripotent stem cell- (PSC-) derived neural stem cell (NSC) line expressing NGN2, and ASCL1 under an inducible system; (b) culturing the NSC line under conditions to induce the expression of NGN2 and ASCL1, in combination with a cell cycle inhibitor for at least about 7 days, thereby generating PSC-derived neurons; (c) replating the PSC-derived neurons in presence of primary human astrocytes; (d) differentiating and maturing the PSC-derived neurons for at least about 60 to about 90 days in an automated cell culture system.

In some aspects, the disclosure provides a homogenous population of terminally differentiated neurons derived from pluripotent stem cells, wherein at least 95% of the neurons express: Map2; Synapsin 1 and/or Synapsin 2; and beta-III tubulin. In some aspects, provided is a homogenous population of terminally differentiated neurons derived from pluripotent stem cells, wherein: (a) at least 95% of the neurons express one or more pre-synaptic markers selected from vGLUT2, Synapsin 1, and Synapsin 2; and/or (b) at least 95% of the neurons express one or more post-synaptic markers selected from: PSD95, SHANK, PanSHANK, GluR1, GluR2, PanSAPAP, and NR1; and/or (c) at least 100 postsynaptic endings of a neuron overlap with presynaptic endings of other neurons and/or at least 100 presynaptic endings of the neuron overlap with postsynaptic endings of other neurons. In some embodiments, at least 95% of the neurons express: two or more pre-synaptic markers selected from: vGLUT2, Synapsin 1, and Synapsin 2; and/or two or more post-synaptic markers selected from: PSD95, SHANK, PanSHANK, GluR1, GluR2, PanSAPAP, and NR1. In some embodiments, at least 95% of the neurons express one or more upper-layer cortical neuron markers, optionally wherein no more than 5% of the neurons express one or more lower layer cortical neuron markers. In some embodiments, at least 95% of neurons express CUX2, optionally wherein no more than 5% of neurons express CTIP2 or SATB2. In some embodiments, the process of deriving terminally differentiated neurons from pluripotent stem cells comprises: (a) generating a pluripotent stem cell- (PSC-) derived neural stem cell (NSC) line expressing NGN2, and ASCL1 under an inducible system; (b) culturing the NSC line under conditions to express NGN2 and ASCL1, in combination with cell cycle inhibitor for at least about 7 days, thereby generating PSC-derived neurons; (c) replating the PSC-derived neurons in presence of primary human astrocytes; (d) differentiating and maturing the PSC-derived neurons for at least about 60 to about 90 days in an automated cell culture system. In some embodiments, the neurons express representative markers for dendrites, cell bodies, axons and synapses in highly replicable manner. In some embodiments, the expressions of dendritic marker MAP2, cell body marker CUX2, axon marker Tau, and synapse marker Synapsin 1/2 in neurons are highly replicable across replicate experiments, wherein the z-factor for each of MAP2, CUX2, Tau and Synapsin 1/2 is at least 0.4.

In some aspects, the disclosure provides a pluripotent stem cell-derived neuronal culture system for use in modeling neurodegenerative diseases, wherein the culture system comprises substantially defined culture media and wherein the culture system is amenable to modular and tunable inputs of: one or more disease-associated components and/or one or more neuroprotective components. In some embodiments, the neurodegenerative disease is Alzheimer's disease, wherein: (a) the disease-associated components comprises soluble Aβ species; (b) the disease-associated component comprises overexpression of mutant APP, optionally wherein the disease-associated component comprises inducible overexpression of mutant APP; (c) the disease-associated component comprises pro-inflammatory cytokine; (d) the neuroprotective component comprises anti-Aβ antibody; (e) the neuroprotective component comprises DLK inhibitor, GSK3β inhibitor, CDK5 inhibitor, and/or Fyn kinase inhibitor; and/or (f) the neuroprotective component comprises microglia. In some embodiments, the system does not comprise matrigel. In some embodiments, the system comprises completely defined culture media and/or matrices. In some embodiments, the soluble Aβ species comprises soluble Aβ oligomers and/or soluble Aβ fibrils.

In some embodiments according to any one of the neuronal culture systems described herein, the neuronal culture system comprises the disease-associated component comprising soluble Aβ species, wherein: Tau protein in the neuronal culture is hyperphosphorylated in one or more of S396/404, S217, S235, S400/T403/S404, and T181 residues. In some embodiments, the culture system comprises the one or more disease-associated components comprising soluble Aβ species, wherein: the neuronal culture system displays increased neuronal toxicity as compared to a corresponding neuronal culture system not comprising the soluble Aβ species. In some embodiments, the neuronal culture system comprises the disease-associated component comprising soluble Aβ species, wherein: the culture system displays a decrease of MAP2-positive neurons as compared to a corresponding neuronal culture system not comprising the soluble Aβ species. In some embodiments, the neuronal culture system comprises the disease-associated component comprising soluble Aβ species, wherein: the culture system displays a decrease of synapsin-positive neurons as compared to neuronal culture system not comprising the soluble Aβ species. In some embodiments, the neuronal culture system comprises the disease-associated component comprising soluble Aβ species, wherein: the neuronal culture system displays an increase in Tau phosphorylation in neurons as compared to a neuronal culture system not comprising the soluble Aβ species, wherein the concentration of Aβ is no less than a first concentration; the neuronal culture system displays a decrease of synapsin-positive neurons as compared to a neuronal culture system not comprising the soluble Aβ species, wherein the concentration of Aβ is no less than a second concentration; the culture system displays a decrease of CUX2-positive neurons as compared to neuronal culture system not comprising the soluble Aβ species, wherein the concentration of Aβ is no less than a third concentration; and the culture system displays a decrease of MAP2-positive neurons as compared to neuronal culture system not comprising the soluble Aβ species, wherein Aβ is at no less than a forth concentration. In some embodiments, the first concentration is higher than the second, third and fourth concentrations; and/or the second concentration is higher than the third and fourth concentrations; and/or the third concentration is higher than the fourth concentration. In some embodiments, the first concentration is about 5 μM, the second concentration is about 2.5 μM, the third concentration is about 1.25 μM and the fourth concentration is about 0.3 μM.

In some embodiments according to any one of the neuronal culture system described herein, the neuronal culture system comprises the disease-associated component comprising soluble Aβ species, wherein: the neuronal culture system further comprises astrocytes in co-culture, wherein the astrocytes exhibit increased GFAP expression and/or the astrocytes exhibit increased GFAP fragmentation as compared to astrocytes co-cultured in a neuronal culture system not comprising the soluble Aβ species. In some embodiments, the neuronal culture system comprises the disease-associated component comprising soluble Aβ species, wherein: the neuronal culture system exhibits Methoxy X04-positive Aβ plaques or plaque-like structures. In some embodiments, the neuronal culture system exhibits neuritic dystrophy. In some embodiments, at least a subset of the Methoxy X04-positive Aβ plaques or plaque-like structures are surrounded by neurites, optionally wherein the neurites are marked by neurofilament heavy chain (NFL-H) axonal swelling and/or phosphorylated Tau (S235) positive blebbings, further optionally wherein the neurites are dystrophic. In some embodiments, the plaques or plaque-like structures surrounded by neurites exhibit: ApoE expression localized in the amyloid plaques and/or APP in the membranes of the neurites.

In some embodiments according to any one of the neuronal culture systems described herein, the culture system comprises: the disease-associated component comprising soluble Aβ species, the disease-associated component comprising neuroinflammatory cytokine, and the neuroprotective component comprising microglia. In some embodiments, the microglia is iPSC-derived microglia and expresses one or more of: TREM2, TMEM 119, CXCR1, P2RY12, PU.1, MERTK, CD33, CD64, CD32 and IBA-1. In some embodiments, the neuronal culture system comprising (1) soluble Aβ species, and (2) microglia exhibits decreased neuronal toxicity as compared to a corresponding neuronal culture system not comprising microglia. In some embodiments, the neuronal culture system comprising (1) soluble Aβ species, and (2) microglia exhibits increased microglial-Aβ plaque association and/or increased Aβ plaque formation as compared to a corresponding neuronal culture system not comprising microglia. In some embodiments, the neuronal culture system comprising (1) soluble Aβ species, (2) neuroinflammatory cytokine and (3) microglia exhibits less than 10% change in neuronal toxicity as compared to a corresponding neuronal culture system not comprising microglia. In some embodiments, the neuronal culture system comprising (1) soluble Aβ species, (2) neuroinflammatory cytokine and (3) microglia exhibits increased microglial-sAβ plaque association and/or increased sAβ plaque formation as compared to a corresponding neuronal culture system not comprising microglia. In some embodiments, the neuronal culture system comprises the disease-associated component comprising (1) the disease-associated component comprising soluble Aβ species, and (2) the neuroprotective component comprising microglia. In some embodiments, the neurons exhibit one or more of DLK, GSK3, CDK5, and Fyn kinase signaling.

In some embodiments according to any one of the neuronal culture systems described herein, the neuronal culture comprises homogenous and terminally differentiated neurons from pluripotent stem cells, wherein the homogenous and terminally differentiated neurons from pluripotent stem cells are generated in a process comprising the steps of: (a) generating a pluripotent stem cell- (PSC-) derived neural stem cell (NSC) line expressing NGN2, and ASCL1 under an inducible system; (b) culturing the NSC line under conditions to induce the expression of NGN2 and ASCL1, in combination with a cell cycle inhibitor for at least about 7 days, thereby generating PSC-derived neurons; (c) replating the PSC-derived neurons in presence of primary human astrocytes; (d) differentiating and maturing the PSC-derived neurons for at least about 60 to about 90 days in an automated cell culture system.

In some embodiments according to any one of the homogenous populations, methods or neuronal culture systems described herein, the step of differentiating and maturing the PSC-derived neurons comprises one or more rounds of automated culture media replacements; and wherein the automated cell culture system sustains differentiation, maturation and/or growth of neuronal cells for at least about any one of: 30, 60, 80, 90, 120, or 150 days. In some embodiments, the automated culture media replacement comprises automated culture media aspiration and automated culture media replenishment; and/or wherein the cell culture system comprises one or more 384-well plates. In some embodiments, the automated culture media aspiration comprises aspiration with a pipet tip, wherein: (a) the distal end of the pipet tip is at about 1 mm above the bottom surface of the well before, during and/or after the aspiration; (b) the pipet tip is at an angle of about 90° to the bottom surface of the well before, during and/or after the aspiration; (c) the pipet tip has a displacement of no more than 0.1 mm from the center of the well before, during and/or after the aspiration; optionally wherein the pipet tip is at the center of the well before, during and/or after the aspiration (no displacement); (d) the speed of media aspiration is no more than about 7.5 μl/s; (e) the start of media aspiration is about 200 ms subsequent to the pipet tip being placed 1 mm above the bottom surface of the well (f) the pipet tip is inserted into the well at a speed of about 5 mm/s prior to aspiration; and/or (g) the pipet tip is withdrawn from the well at a speed of about 5 mm/s after aspiration.

In some embodiments according to any one of the homogenous populations, methods or neuronal culture systems described herein, the automated culture media replenishment comprises dispensing media with a pipet tip, wherein: (a) the distal end of the pipet tip is at about 1 mm above the bottom surface of the well before the dispensing; (b) the distal end of the pipet tip is withdrawn from the well at about 1 mm/s during the dispensing; (c) the pipet tip is at an angle of about 90° to the bottom surface of the well before and/or during the dispensing; (d) the pipet tip has a displacement of no more than 0.1 mm from the center of the well before, and/or during the dispensing, optionally wherein the pipet tip is at the center of the well before, and/or during the dispensing (no displacement); (e) the pipet tip is displaced to contact a first side of the well about 1 mm from the center in a first direction, at a height of about 12.40 mm above the bottom of the well at a speed of about 100 mm/s; (f) the pipet tip is displaced to contact a second side of the well about 1 mm from the center in a second direction, at a height of about 12.40 mm above the bottom of the well at a speed of about 100 mm/s, optionally wherein the first direction is at an angle of about 1800 to the second direction; (g) the speed of media dispensing is no more than about 1.5 μl/s; (h) the acceleration of media dispensing is about 500 μl/s²; (i) the deceleration of media dispensing is about 500 μl/s²; (j) the start of media dispensing is about 200 ms subsequent to the pipet tip being placed 1 mm above the bottom surface of the well; (k) the pipet tip is inserted into the well at a speed of about 5 mm/s prior to dispensing; and/or (l) the pipet tip is withdrawn from the well at a speed of about 5 mm/s after dispensing.

In some embodiments according to any one of the homogenous populations, methods or neuronal culture systems described herein, the cell culture system comprises a 384-well plate; further wherein: (a) the automated cell culture system comprises automated discarding of a used rack of 384-pipet tips and automated engagement of a new rack of 384-pipet tips subsequent to each round of media aspiration; and/or (b) the automated cell culture system comprises automated discarding of a used rack of 384-pipet tips and automated engagement of a new rack of 384-pipet tips subsequent to each round of media dispensing. In some embodiments, the cell culture system comprises one or more batches of 384-well plates, wherein each batch comprises up to twenty-five 384-well plates arranged in 5 columns and 5 rows; further wherein: (a) the automated cell culture system comprises automated discarding of up to 25 corresponding used racks of 384-pipet tips and automated engagement of up to 25 corresponding new racks of 384-pipet tips subsequent to each round of media aspiration; and/or (b) the automated cell culture system comprises automated discarding of up to 25 corresponding used racks of 384-pipet tips and automated engagement of up to 25 corresponding new racks of 384-pipet tips subsequent to each round of media dispensing.

In some embodiments according to any one of the homogenous populations, methods or neuronal culture systems described herein, (a) the time period between two rounds of culture media replacements are about any one of: 1, 2, 3, 4, 5, 6, 7, 8, 9, or 10 days; and/or (b) about any one of: 30%, 40%, 50%, 60%, 70%, or 80% of culture media is replaced in one or more rounds of culture media replacement. In some embodiments, (a) the time period between two rounds of culture media replacements are about 3 or 4 days; and/or (b) about 50% of culture media is replaced in one or more rounds of culture media replacement.

In some aspects, provided is a method of screening compounds that increase neuroprotection, comprising: contacting the compound with the neuronal culture in any one of the neuronal culture systems described, and quantifying improvements in neuroprotection. In some embodiments, the improvements in neuroprotection comprises: increase in amounts of one or more of: dendrites, synapses, cell counts, and/or axons in the neuronal culture. In some embodiments, the method comprises quantifying the increase in amounts of one or more of: dendrites, synapses, cell counts, and/or axons in the neuronal culture, wherein: (a) the amount of dendrites is measured by levels of MAP2 in the neuronal culture; (b) the amount of synapses is measured by levels of Synapsin 1 and/or Synapsin 2 in the neuronal culture; (c) the amount of cell counts is measured by levels of CUX2 in the neuronal culture; and/or (d) the amount of axons is measured by levels of beta III tubulin in the neuronal culture. In some embodiments, a compound is selected for further testing if: (a) the level of MAP2 in the neuronal culture is increased by ≥30%; (b) the level of Synapsin 1 or Synapsin 2 is increased by ≥30%; (c) the level of CUX2 is increased by ≥30%; and/or (d) the level of beta III tubulin is increased by ≥30%; when compared to a corresponding neuronal culture not contacted with the compound. In some embodiments, a compound is determined to be neuroprotective if: (a) the level of MAP2 in the neuronal culture is increased by ≥30%; (b) the level of Synapsin 1 or Synapsin 2 is increased by ≥30%; (c) the level of CUX2 is increased by ≥30%; and/or (d) the level of beta III tubulin is increased by ≥30%; when compared to a corresponding neuronal culture not contacted with the compound.

### BRIEF DESCRIPTION OF THE DRAWINGS

Representative embodiments of the invention are disclosed by reference to the following figures. It should be understood that the embodiments depicted are not limited to the precise details shown.

The patent or application file contains at least one drawing executed in color. Copies of this patent or patent application publication with color drawing(s) will be provided by the Office upon request and payment of the necessary fee.

FIG. 1A shows a schematic workflow of human induced pluripotent stem cell (iPSC) neuron differentiation, plating, maintenance, and maturation with automated media change using Fluent® liquid handler (Tecan). Mature culture (12 weeks or older) is ready for various experimental treatment and conditions. At the end of experiments, fixed cells are processed for immunostaining using automated plate washers, and then quantified with high content image analysis via IN Cell Analyzer 6000 (GE).

FIG. 1B shows representative images of unsynchronized, heterogeneous wild-type (WT) iPSC-derived neuronal stem cells (NSC) differentiation (red arrows indicate differentiated neurons; green arrows indicate undifferentiated NSCs). Scale Bar=50 μm.

FIG. 1C shows the stable expression of cumate-inducible NGN2/ASCL1/GFP (NAG) construct and treatment with cell cycle inhibitors synchronizes and homogenizes human iPSC neuron differentiation. Scale Bar=50 μm.

FIGS. 1D-1J show a representative work-flow of the high throughput, automated human iPSC-derived neuron differentiation and culturing platform. FIG. 1D shows a 20 culture plate media change using the Fluent© Automated Workstation (Tecan). FIG. 1E shows the Fluent© 384 tip liquid handler head, that consistently and systematically removes old media and adds new media across all wells per plate. FIG. 1F shows the integrated incubator and barcoded plates enable automated plate tracking and care. FIG. 1G shows the automated plate ejection from the integrated incubator of FIG. 1F. FIG. 1H shows the gripper arm retrieving the plate of FIG. 1G. FIG. 1I shows the gripper arm of FIG. 1H placing the plate of FIG. 1G on the plate deck for subsequent media change. FIG. 1J shows the gripper arm removing the lid and placing it on the plate lid hotel during media change.

FIG. 1K shows that differentiated NAG neurons express dendritic marker MAP2 (red), layer II/III cortical marker CUX2 (green), with a small subpopulation expressing layer V/VI marker CTIP2 (blue) indicated by white arrows. Scale Bar=50 μm.

FIGS. 1L-1R show that mature NAG neurons express various cell markers: MAP2 (blue), synaptic markers VGLUT2 (red) and Shank (green), Scale Bar=20 μm (FIG. 1L); Synapsin (red) and PSD95 (green), Scale Bar=10 μm (FIG. 1M); Pan SHANK (green), Scale Bar=10 μm (FIG. 1N); Pan-SAPAP (green), Scale Bar=10 μm (FIG. 1O); GluR1 (green), Scale Bar=10 μm (FIG. 1P); GluR2 (green), Scale Bar=10 μm (FIG. 1Q); and NR1 (green), Scale Bar=10 μm (FIG. 1R).

FIG. 1S shows a schematic illustrating that high content image analyses are made from 9 fields/well in a 384-well plate covering 70% well area.

FIGS. 1T-1Y show exemplary image analysis using the IN Cell Developer toolbox companion software to quantitate phenotypes in an automated, systematic and unbiased way. Precise scripts were developed to isolate exact regions of interest, which are shown in red on the right panels. Multiple measurements such as total area, total intensity and count are made for each markers. Representative images of the cellular phenotypes include those of dendrites (FIG. 1T-1U), synapses (FIG. 1V-1W), and axons (FIG. 1X-1Y).

FIG. 1Z shows Z-factors that were calculated from the results of FIGS. 1T-1Y using the neuron culturing platform and high content image analysis software. Z-factors are in the range from 0.5-0.75, and averaged from 10-20 different experiments using different batches of neurons. Each experiment with four wells, 1,000+ neurons/well quantified. Error bars+/−s.e.m. and n=4 wells.

FIG. 2A shows a schematic depicting the process of soluble Aβ species generation. Soluble Aβ species were generated by resuspending lyophilized Aβ42 monomers in PBS and incubating monomers at 4 C for 14, 24, 48, 72 hours then frozen to stop the oligomerization process.

FIGS. 2B-2D show dendrite toxicity (MAP2) (FIG. 2B), synapse loss (Synapsin 1/2) (FIG. 2C), and p-Tau induction (S396/S404) (FIG. 2D) of Aβ42 monomers oligomerized for 14, 24, 48, and 72 hours. Error bars+/−s.e.m. and n=4 wells.

FIGS. 2E-2G show the characterization of soluble Aβ species oligomerized for 24 hours for oligomeric and fibril conformation using Aβ oligomer selective and Aβ fibril selective ELISA assays. FIG. 2E shows a 6E10-6E10 assay utilizing the same anti-AP42 (6E10) for capture and detection to selectively bind to oligomeric Aβ42 species. FIG. 2F shows a T622-6E10 oligomer assay uses Aβ oligomer specific antibody clone GT622 as capture and pan Aβ antibody clone (6E10) as detection. FIG. 2G shows a OC-6E10 assay uses Aβ fibril selective antibody clone OC as capture and pan Aβ antibody clone (6E10) as detection. All values were normalized to Aβ42 monomer negative control, and Aβ42 fibrils were generated by oligomerization in 37° C. as a positive control to demonstrate specificity of this assay.

FIGS. 2H-2J show dendrite toxicity (MAP2) (FIG. 2H), synapse loss (Synapsin 1/2) (FIG. 2I), and p-Tau induction (5396/5404) (FIG. 2J) of Aβ42 monomers and scramble control tested at 0, 2.5, 5 μM for dose response. Error bars+/−s.e.m. and n=4 wells.

FIG. 2K shows an exemplary image of rat cortical neurons treated with 5 μM soluble Aβ species for 7 days. The rat neurons form many plaque-like, Methoxy-X04 positive structures (blue), and a few of these plaque-like structures are surrounded by dystrophic neurite-like blebbings of NFL-H (green), and phospho-Tau (AT270, red). Neuritic plaques are indicated by dotted white boxes. Scale Bar==100 μM.

FIGS. 2L-2M show zoomed in images of FIG. 2K, showing axonal swelling (NFL-H; green) and p-Tau induction (S235; red) in axons around AD-plaque structures (Methoxy-X04; blue). The extent of the neuritic dystrophy is significantly less than that of iPSC human neurons in the same amount of time (7 Days). Scale Bar=20 μM.

FIGS. 2N-2O show that rat neurons fail to show Aβ42 oligomer toxicity in response to many lots of Aβ42 oligomer preparations in comparison to human neurons in terms of dendrite (MAP2) loss (FIG. 2N) and severe synapse loss (Synapsin 1/2) (FIG. 2O). Error bars+/−s.e.m. and n=4 wells.

FIGS. 3A-3B show that differentiated NAG neurons (12 weeks+) show loss of dendrites (MAP2, green) and cell bodies (CUX2, red) when treated with soluble A1 species for 7 days (FIG. 3B) in comparison to no treatment condition (FIG. 3A).

FIG. 3C shows that anti-Aβ antibody co-treatment with soluble Aβ species blocks Aβ-induced cell death. Scale Bar=50 μm.

FIG. 3D shows dose-dependent, progressive differentiated NAG neuron cell death, as quantified by the percentage of cell body (CUX2) numbers in AD-treated normalized to an untreated control.

FIG. 3E shows dose-dependent, progressive dendritic (MAP2) loss, as quantified by percentage of MAP2 area in AD-treated differentiated NAG neurons, normalized to an untreated control.

FIGS. 3F-3G show that Aβ42 treatment of differentiated NAG neurons induces phosphorylation of Tau (p-Tau 396-404, white) and mislocalization to the cell body.

FIG. 3H shows that anti-Aβ antibody co-treatment of differentiated NAG neurons with sAβ42s blocks AD-induced Tau hyperphosphorylation. Scale Bar=50 μm.

FIG. 3I shows a dose-dependent, and time course of phosphorylation of tau at S396/404 in differentiated NAG neurons. Phospho-Tau induction increased at 5 μM Aβ treatment before a decrease associated with cell death occurred as quantified by fold p-tau 396/404 staining in AD-treated differentiated NAG neurons, normalized to untreated control.

FIGS. 3J-3K show that Aβ42 treatment of differentiated NAG neurons causes synapse loss in neurons (synapsin, green).

FIG. 3L shows that anti-Aβ antibody co-treatment of differentiated NAG neurons with sAβ42s blocks synapse loss phenotype. Scale Bar=5 μm.

FIG. 3M shows a dose-response and time course of synapse (synapsin 1/2) loss in Aβ-treated differentiated NAG neurons culture normalized, to untreated control.

FIG. 3N-O show that sAβ42s treatment of differentiated NAG neurons induces axon fragmentation (beta-3 tubulin Tuj1, white).

FIG. 3P shows that anti-Aβ antibody co-treatment of differentiated NAG neurons blocks axon fragmentation. Scale Bar=50 μm.

FIG. 3Q shows dose-response and a time course of axon fragmentation as quantified by percentage of axon (NFL-H) area in AD-treated differentiated NAG neurons, normalized to an untreated control.

FIG. 3R shows that anti-Aβ antibody treatment of differentiated NAG neurons rescues all three markers in a dose dependent manner and IC50 curves can be drawn and calculated (IC50 curve fitted by Prism software). Error bars+/−s.e.m. and n=4 wells.

FIGS. 4A-4D show that 5 μM Aβ42 treatment of differentiated NAG neurons induces somatodendritic accumulation of tau (overlap with MAP2, third panel) and phosphorylation at S202/T205 and as detected by AT8 antibody (green). Scale Bar=50 μm.

FIGS. 4E-T show the staining of Tau phosphorylation site S217 (FIGS. 4E-H), site S235 (FIGS. 4I-4L), site S400/T403/S404 (FIGS. 4M-4P), and site T181 (AT270) (FIGS. 4Q-4T), of 5 μM Aβ42 treated differentiated NAG neurons. Scale Bar=50 μm.

FIGS. 4U-4Y show the quantification of induction of phosphorylated Tau of AP42 treated differentiated NAG neurons, which increases in dose response to Aβ treatment concentration as specified. The induction fold was calculated by ratio of p-Tau area to total Tau (H1T7) area in AP treated induction over ratio of p-Tau area to total Tau (HT7) in untreated control. Error bars+/−s.e.m. and n=4 wells.

FIG. 4Z shows western blot images showing soluble (right) and insoluble (left) fractions of protein lysates obtained from iPSC neurons and astrocytes treated with 0, 0.3, 0.6, or 1.25 μM sA342s twice weekly for three weeks, then probed for 3R Tau protein, total Tau (HT7) and loading control histone H3. Upon treatment with soluble A1 species, there is a dose dependent increase in the insoluble 3R and total Tau and depletion of these proteins from the soluble fraction. In high concentrations of soluble Aβ species, there are lower molecular weight truncated Tau proteins (red asterisks) and larger molecular weight Tau aggregates (black asterisks).

FIGS. 5A-5B show representative images of iPSC derived neurons and primary astrocytes that were treated with 2.5 μM soluble Aβ species for 7 days, and stained for A3-plaque structures. FIG. 5A shows Methoxy-X04; blue and 6E10 (AD; green), and FIG. 5B shows axons (NFL-H; green) and p-Tau (S235; red), with neuritic plaques indicated by dotted white boxes.

FIGS. 5C-5E show zoomed in images of B showing axonal swelling (NFL-H; green) and p-Tau induction (S235; red) in axons around AD-plaque structures (Methoxy-X04; blue).

FIGS. 5F-5K show representative images of neurons that were treated with 2.5 μM soluble Aβ species and analyzed over a 21-day time course for axonal fragmentation (NFL-H; green), p-Tau induction (S235; red), and plaque formation (Methoxy-X04; blue). Dystrophic neurites composed of NFL-H and p-Tau swellings surrounding X04-positive Aβ-plaques were observed. Scale Bar=50 μm.

FIGS. 5L-5N show the phenotypes of neuronal cultures that were treated with soluble Aβ species at concentrations of 5 μM (red), 2.5 μM (orange), 1.25 μM (green), 0.6 μM (blue) and 0.32 μM (purple) on day 0. Neurons were subsequently fixed at day 1, 3, 7, 10, 14, and 21, and stained for various markers. Plaque formation (Methoxy-X04 dye positive regions) begins early after Aβ oligomer treatment and total plaque area (FIG. 5L) increases with high Aβ oligomer concentrations and over time while average plaque area (FIG. 5M) stays relatively consistent over time. Neurons exhibit dystrophic neurite formation (as measured by S235 p-Tau and NFL-H positive axon area), and these neuritic plaques increase in number with high Aβ oligomer concentrations and over time (FIG. 5N). Error bars+/−s.e.m. and n=4 wells.

FIG. 5O shows a schematic showing a summary of hypothesized sequential events of neurodegeneration, plaque, and dystrophic neurite formation.

FIGS. 6A-D shows stained AD-plaque structures (Methoxy-X04; blue), axons (NFL-H; green), and p-Tau (AT270; red) of NAG-NSC Line 2 and primary astrocytes treated with 5 μM soluble Aβ species for 7 days. FIGS. 6C and D each shows a zoomed in image of a neuritic plaque. Scale Bar=50 μm.

FIG. 6E shows loss of dendrites (MAP2, blue) and loss of synapses (synapsin, green) of NAG-NSC Line 2 and primary astrocytes treated with 5 μM soluble Aβ species for 7 days, compared to no treatment control on right.

FIGS. 6F and 6K show the quantification of MAP2 and synapsin in NAG-NSC Line 2 and primary astrocytes treated with 5 μM soluble Aβ species for 7 days, respectively. The results show dose-dependent and time-dependent loss of dendrites (MAP2) and synapses (synapsin), and both can be rescued with treatment with anti-Aβ antibody (Crenezumab).

FIGS. 6I-J show loss of dendrites (MAP2, blue), Tau fragmentation (HT7, red), as well as upregulation and mislocalization of phospho-Tau (pS396-404, green) from axons to cell bodies and dendrites (FIG. 6J), of NAG-NSC Line 2 and primary astrocytes treated with 5 μM soluble Aβ species for 7 days.

FIGS. 6L-6M show the phospho-Tau p396-404 (FIG. 6L) and phospho-Tau p400-403-404 (FIG. 6M) fold induction, illustrating that phospho-Tau are upregulated in a dose and time-dependent manner, and that this can be blocked with the treatment of anti-A antibody (Crenezumab).

FIGS. 7A-7C show that primary human astrocytes cultured alone in Neuron Maintenance Medium express astrocyte markers GFAP (green), Vimentin (red, FIG. 7A), ALDH1L1 (red, FIG. 7B), and EAAT1 (red, FIG. 7C). Scale Bar=100 μm.

FIG. 7D shows that primary human astrocytes cocultured with neurons in Neuron Maintenance Medium develop elaborate processes and more mature morphology (GFAP, white). Scale Bar=100 μm.

FIG. 7E shows that primary human astrocytes cultured alone in Neuron Maintenance Medium upregulated GFAP (right, white; left, green), starting at 3 divisions (3DIV) upon treatment with 5 μM soluble Aβ species, aggregate Aβ (6E10, blue), and form diffuse dye-positive structures (Methoxy-X04, red) that are morphologically different from dye-positive structures that microglia form. At 1DIV (top), we observe small aggregates of Aβ around cell processes that grow and begin to result in some cell death, which worsens at 7 divisions (7DIV). Yellow arrows indicate astrocytes with increased GFAP expression. Red arrows indicate dead/dying cells. White dotted box indicates zoomed in region on the right. Scale Bar=100 μm.

FIG. 7F shows the quantification of the average GFAP intensity/cell (primary human astrocytes cultured alone), which shows that at 3DIV astrocytes treated with soluble Aβ species upregulate GFAP, and this is blocked by treatment with anti-Aβ antibody (Crenezumab). Error bars+/−s.e.m. and n=4 wells; ANOVA ****P<0.0001, ***P<0.001, **P<0.01.

FIG. 7G shows cell death quantified by fragmentation of the cell body (primary human astrocytes cultured alone) using GFAP shows that primary human astrocytes treated with soluble Aβ species show marked cell death at 3DIV which worsens at 7DIV. Error bars+/−s.e.m. and n=4 wells; ANOVA ****P<0.0001, ***P<0.001, **P<0.01.

FIGS. 7H-7J show that primary human astrocytes cocultured with neurons treated with 5 μM soluble Aβ species also demonstrate similar upregulation of GFAP (FIG. 71) and cell fragmentation indicating cell death (FIG. 7J) in a dose- and time-dependent manner. Error bars+/−s.e.m. and n=4 wells; ANOVA ****P<0.0001, ***P<0.001, **P<0.01. Scale Bar=100 μm.

FIGS. 8A-8E show iPSC derived microglia stained with antibodies against microglia markers: TREM2, TMEM119, CXCR1, P2RY12, PU.1 (green); MERTK, CD33, CD64, CD32 (red); IBA1 (blue). The results show that human iPSC microglia express common microglial markers and have typical ramified morphology. Scale Bar=50 μm.

FIGS. 9A-9B show representative images empty wells (FIG. 9A; Scale Bar=20 μm) or 12 week old iPSC neurons (FIG. 9B; Scale Bar=50 μm) treated with soluble Aβ species at the indicated concentrations, and stained with X04 (blue), AP (green), NFL-H (green) and p-Tau S235 (red). Empty wells show AP precipitates but no X04 positive structure (FIG. 9A). In iPSC neuron wells, a dose dependent increase of X04 staining is shown (FIG. 9B). A subset of X04 are also surrounded by dystrophic neurites (NFL-H and S235 positive axonal swellings).

FIG. 9C shows representative images of microglia treated with soluble Aβ species ranging from 0-5 μM, and also treated in combination with INFγ. The bottom panel shows a zoomed in section. Aβ plaques are stained by X04 (blue), microglia are labeled with Actin (green), and IBA1 (red). Scale Bar=50 μm.

FIG. 9D shows representative images from indicated conditions of neuron and astrocytes co-culture, and tri-culture of neurons, astrocytes, and microglia treated with soluble Aβ species with or without pro-inflammatory cytokine combination (IFNγ+IL1b+LPS). The bottom panel shows a zoomed in section. Aβ plaques were stained with X04 (blue), dystrophic neurites swellings were stained with NFL-H (green), and microglia were labeled with IBA1 (red). In triple culture, Aβ oligomer addition led to Aβ plaque formations surrounded by dystrophic neurites and encircled by microglia similar to plaque presentation in vivo. Scale Bar=20 μm.

FIGS. 9E-9F show that IFNγ increases plaque formation and plaque interaction as quantified from the images shown in FIG. 9C. FIG. 9E shows the quantification of X04 intensity, and FIG. 9F shows quantification of IBA1 number of the images shown in FIG. 9C. Error bars+/−s.e.m. and n=4 wells; ANOVA ****P>0.0001.

FIG. 9G shows the quantification of the area of IBA1 overlap with X04 in FIG. 9D. Pro-inflammatory cytokines increased microglia association with plaque. Error bars+/−s.e.m. and n:=4 wells; ANOVA ****P>0.0001.

FIG. 9H shows the quantification of the total area of X04 staining in FIG. 9D. Microglia increased the X04 plaque area, and proinflammatory cytokine addition increased the plaque area furthermore. Error bars+/−s.e.m. and n=4 wells; ANOVA ****P>0.0001.

FIG. 9I shows the quantification of the total area of MAP2 staining in FIG. 9D. Aβ oligomer addition caused severe reduction to neurons culture, and microglia culture provided 25% MAP2 protection from Aβ oligomer. This protection is lost when proinflammatory cytokine is added. Error bars+/−s.e.m. and n=4 wells; ANOVA ****P>0.0001.

FIG. 10 shows that (left) human iPSC-derived microglia (IBA1, red) receiving no treatment show no accumulation of Aβ (6E10, blue), no plaque-like structures (Methoxy-X04, green). The middle panel shows that human iPSC-derived microglia (IBA1, red) treated with 2.5 μM soluble Aβ species (6E10, blue) show accumulation of discrete plaque-like structures (Methoxy-X04, green) that are surrounded by cells. The right panel shows HeLa cells (Phalloidin, red) treated with 2.5 μM soluble Aβ species (6E10, blue) show low surface binding of AD, but do not demonstrate discrete plaque-structures (Methoxy-X04, green) observed in human iPSC derived-microglia. Overall, FIG. 10 shows that amyloid plaque-like structures are generated by human iPSC microglia but not by HeLa cells.

FIGS. 11A-11D show synapse % rescue versus MAP2% rescue (FIGS. 11A-11B) and beta III tubulin % rescue versus MAP2% rescue (FIGS. 11C-11D), in neurons and astrocytes (FIGS. 11A and 11C) or neurons, astrocytes, and microglia (FIGS. 11B and 11D) treated with 5 μM sAβ42s and small molecules from a focused screen of known neuroprotective agents at multiple concentrations (50 μM, 25 μM, 12.5 μM and 6.25 μM (double culture), 50 μM, 12.5 μM, 3.1 μM and 0.78 μM (triple culture). Small molecules that prevented toxicity in dendrites (MAP2), synapses (Synapsin 1/2), cell count (CUX2), or axons (NFL-H) at or above 30% were considered hits (red dotted line). An anti-Aβ antibody was used as a positive control that prevented all types of toxicity.

FIGS. 11E-11G show the further validation of the top hits DLKi (FIG. 11E), Indirubin-3′-monoxime (FIG. 11F), and AZD0530 (FIG. 11G) from focused screen by IC50 curves, against MAP2 (blue), Synapsin 1/2 (green), CUX2 (red), and NFL-H (purple). Error bars+/−s.e.m. and n=4 wells. IC50 curves were fitted by Prism software.

FIG. 11H shows that Aβ42 oligomer treatment induced expression of p-cJun (green) in nucleus (HuCD, red). Scale Bar=50 μm.

FIG. 11I shows the quantification of MAP2 (blue), HuC/D (red), p-c-Jun (green) staining. The results indicate an increase in c-Jun phosphorylation with prolonged Aβ42 oligomer treatment. Error bars+/−s.e.m. and n=4 wells.

FIG. 11J shows that 22 week old iPSC neuron culture treated with Aβ42 oligomer displays a dose-dependent, sustained phosphorylation of c-Jun as shown by western blot. GAPDH served as a loading control.

FIG. 11K shows the quantification of the western blot from FIG. 11J. p-c-Jun induction was normalized to GAPDH. Error bars+/−s.e.m. and n=4 wells.

FIGS. 11L-11O show that the inhibition of known components of the DLK-JNK-c-Jun pathway, using small molecules VX-680 (FIG. 11L), GNE-495 (FIG. 11M), PF06260933 (FIG. 11N), and JNK-IN-8 (FIG. 11O), prevents Aβ42 oligomer-induced neural toxicity in all measured markers in a dose-dependent manner. Error bars+/−s.e.m. and n=4 wells. IC50 curves were fitted by Prism software.

FIGS. 12A-G show results where hits from focused screen (FIGS. 11A-110) were tested in dose response curve for markers MAP2 (blue), Synapsin (green), CUX1/2 (red), NF-H (purple). Error bars+/−s.e.m. and n=4 wells. IC50 curves were fitted using Prism software.

FIG. 13A is a schematic showing soluble Aβ species that were made using 5% HiLyte-555 labeled Aβ42 monomers.

FIG. 13B shows representative images taken from Incucyte Zoom software over 7-day time lapse showing the same field of view to track microglial formation of one Aβ42 plaque (red) indicated by white arrow in the indicated time frame. Scale Bar=50 μm.

FIG. 13C shows an exemplary image of microglia movement around the plaques. After 2 days plaque formation has occurred within this 2 hour window, some microglial cells join plaque indicated by yellow arrows and some cells that leave plaque indicated by green arrows. Scale Bar=50 μm.

FIG. 14A shows a schematic depicting soluble Aβ species labeled by HiLyte555 and pHrodo Green continuously fluoresce red, but only fluoresce green under intracellular pH 5 conditions.

FIG. 14B shows quantitative analysis of red Aβ plaque area and green internalized AD. Internalized green Aβ outpace the red extracellular Aβ plaque formation, indicating active Aβ uptake throughout the 7 days and occurring before the appearance of red Aβ plaques.

FIG. 14C shows exemplary images of a plaque formation time lapse movie. Four different plaque formations are retrospectively labelled. Soluble Aβ species are first internalized by microglia (green) before plaque formation (red) in the center of the cultured microglia. Scale Bar=50 μm.

FIG. 14D shows iPSC derived microglia treated with 5 μM soluble Aβ species, and fixed and stained 30 minutes, 6 hours, 1 day, and 4 days following treating. Microglia (IBA1, red) internalize small Aβ puncta (green; white—second row) indicated by white arrowheads (green) after 30 minutes, then externalize these puncta as large aggregates that are faintly X04 positive (blue; white—lower panel) indicated by white arrows, then form large, extracellular X04 positive plaque structures surrounded by microglia from 1-6 days following treatment. Scale Bar=50 μm.

FIG. 14E shows human iPSC-derived microglia treated with 5 μM soluble Aβ species and various dynamin inhibitors (Dynasore, Dynole 4a, Dynole 34-2) at 0.6 μM for 24 h, and plaque-like structures (Methoxy-X04-positive) quantified as percentage of untreated control. Treating with dynamin inhibitors decreased plaque formation approximately 4-fold in all conditions. Error bars+/−s.e.m. and n=4 wells; ANOVA ***P<0.001, **P<0.01.

FIG. 14F shows a summary of proposed step of microglia plaque formation. Error bars+/−s.e.m. and n=4 wells; ANOVA ***P<0.001, **P<0.01.

FIG. 15 shows representative images of human CD14-derived macrophages treated with 5 μM soluble Aβ species, then fixed and stained after 30 minutes, 6 hours, 1 day, and 4 days. The images show that macrophages (IBA1, red) continuously internalize Aβ (green; white—second row) over the course of 4 days and form intracellular X04-positive (blue; white—bottom row) aggregates.

FIGS. 16A-16C show a time course comparison of 12 weeks old iPSC neurons treated with single dose of soluble Aβ species (solid lines) versus repeated dose of Aβ42 at the same concentration (dotted lines), at the indicated concentrations. The MAP2 area (FIG. 16A), synapse count (FIG. 16B), and p-Tau 396-404 induction fold (FIG. 16C) were quantified. Error bars+/−s.e.m. and n=4 wells; ANOVA ****P>0.0001, ***P>0.001, **P>0.01, *P>0.05.

FIG. 16D shows a repeated dosing schedule of 12-week-old iPSC neurons with 0.6 μM of AD. Anti-Aβ antibodies dosing regimens were started at indicated time point. All cells were treated in the same plate and fixed at 21 days post first dose.

FIGS. 16E-16G show the quantified MAP2 area (FIG. 16E), synapsin count (FIG. 16F) and p-Tau induction fold (FIG. 16G) of the treated iPSC neutrons based on the dosing schedule of FIG. 16D. Anti-gD antibodies were dosed similarly to the schedule of FIG. 16D as control (blue bars), along with anti-Aβ antibody (red bar). Error bars+/−s.e.m. and n=4 wells; ANOVA ****P>0.0001, ***P>0.001, **P>0.01, *P>0.05.

FIG. 16H shows a time course study design of anti-A antibodies repeat dosing. Aβ oligomer are added at every indicated timepoint. Anti-Aβ antibodies were added at day 0 (red) as protection model or at day 7 (green) as intervention model. Anti-gD antibodies were used as control (blue).

FIG. 16I shows representative images from the indicated experimental treatments, based on the dosing schedule of FIG. 16H. Neurons were stained for dendrite marker MAP2 (red) and nuclear marker CUX2 (green) at 7DIV and 21DIV. The lower panel shows Aβ plaque staining (X04, white) and p-Tau S235 (red) staining. Scale bar=50 μm. Error bars+/−s.e.m. and n=4 wells.

FIGS. 16J-16K show the quantification of MAP2 area over time (FIG. 16J) and plaque area (FIG. 16K) from the images in FIG. 16I. The results show that the anti-Aβ intervention model is capable of slowing down neuron degeneration and plaque formation.

FIGS. 17A-17C show the quantification of MAP2 area (FIG. 17A), synapsin count (FIG. 17B) and p-Tau induction fold (FIG. 17C), following a repeated dosing schedule of 12-week old human iPSC neuron cultured with twice a week dosed 0.625 μM of soluble Aβ species. 0.625 μM Anti-Aβ antibodies (red) or anti-gD control antibodies (blue) were added at indicated time points for repeated dosing regimens. All cells were treated in the same plate and fixed at 21 days post-first dose.

FIGS. 17D-17F show the quantification of MAP2 area (FIG. 17D), synapsin count (FIG. 17E) and p-Tau induction fold (FIG. 17F), following a repeated dosing schedule of 12-week old human iPSC neuron cultured with twice a week dosed 1.25 μM of soluble Aβ species. 1.25 μM Anti-Aβ antibodies (red) or anti-gD control antibodies (blue) were added at indicated time points for repeated dosing regimens. All cells were treated in the same plate and fixed at 21 days post-first dose.

FIGS. 17G-17I show the quantification of MAP2 area (FIG. 17G), synapsin count (FIG. 17H) and p-Tau induction fold (FIG. 17I), following a repeated dosing schedule of 12-week old human iPSC neuron cultured with twice a week dosed 2.5 μM of soluble Aβ species. 2.5 μM Anti-Aβ antibodies (red) or anti-gD control antibodies (blue) were added at indicated time points for repeated dosing regimens. All cells were treated in the same plate and fixed at 21 days post-first dose.

FIGS. 18A-18B show dendrite protection (MAP2 area) (FIG. 18A) and synapse protection (synapsin count) (FIG. 18B) of iPSC neurons and astrocytes treated with 5 μM soluble Aβ species, followed by serial dilutions of anti-gD and anti-Aβ antibodies with IgG1 and LALAPG backbones, with and without iPSC microglia. Results were analyzed via IC50 curve fitting using Prism software. Microglia provide baseline protection as shown by upward shift in anti-gD graph when microglia are added (gD IgG1 alone, blue; gD IgG1+microglia, dark blue). Anti-Aβ antibody backbones protect dendrites and synapses similarly without microglia (Anti-Aβ IgG1, red; Anti-Aβ LALAPG, green) and with microglia (Anti-Aβ IgG1, dark red; Anti-Aβ LALAPG, dark green). Error bars+/−s.e.m. n=4 wells; ANOVA ****P<0.0001, ***P<0.001, **P<0.01, *P<0.05.

FIGS. 18C-18D show basal dendrite protection (MAP2 area) (FIG. 18C) and plaque formation (Methoxy X04 total intensity) (FIG. 18D) of neuron, astrocyte, microglia triculture treated with 5 μM soluble Aβ species (solid lines) and pro-inflammatory cytokines (dashed lines), followed by the addition of serial dilutions of gD antibody (black lines) and anti-Aβ antibody (red lines). FIG. 18C shows that basal dendrite protection (MAP2 area) is lost in the neuroinflammatory environment, and anti-Aβ treatment shows dose-dependent efficacy. FIG. 18D shows that plaque formation (Methoxy X04 total intensity) increases in pro-inflammatory conditions, however anti-Aβ treatment shows similar plaque reduction. Error bars+/−s.e.m. n=4 wells; ANOVA ****P<0.0001, ***P<0.001, **P<0.01, *P<0.05.

FIG. 18E shows the total Aβ concentration in iPSC microglia (red) treated with 5 μM soluble Aβ species and serial dilutions of anti-A antibody as measured from supernatant; no cells wells were used as control (blue). Anti-Aβ antibody treatment increases soluble Aβ species present in culture supernatant. Error bars+/−s.e.m. n=4 wells; ANOVA ****P<0.0001, ***P<0.001, **P<0.01, *P<0.05.

FIG. 18F shows a summary of sequential events in the iPSC AD model.

### DETAILED DESCRIPTION OF THE INVENTION

In some aspects, provided is a pluripotent stem cell-derived neuronal culture system for use in modeling neurodegenerative diseases (such as Alzheimer's disease), wherein the culture system comprises substantially defined culture media and wherein the culture system is amenable to modular and tunable inputs of one or more disease-associated components and/or one or more neuroprotective components. Also provided are methods of using such a neuronal culture system for use in drug screening and target discovery for neurodegenerative diseases. Further provided are methods of generating homogenous, terminally differentiated neuronal culture from pluripotent stem cells, compositions resulting thereof, and uses of such neuronal culture and compositions for neurodegenerative disease and modeling. In addition, automated cell culture systems that sustain long-term differentiation, maturation and/or growth of neuronal cells are also disclosed, as are the uses of such systems in generating the terminally differentiated neuronal cultures for use in modeling neurodegenerative diseases and drug screening.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N. Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011)

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

As used herein, “treatment” is an approach for obtaining beneficial or desired clinical results. “Treatment” as used herein, covers any administration or application of a therapeutic for disease in a mammal, including a human. For purposes of this invention, beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods of the invention contemplate any one or more of these aspects of treatment.

In the context of neurodegenerative disease, the term “treating” includes any or all of: inhibiting growth of diseased cells, inhibiting replication of diseased cells, lessening of overall disease progression and ameliorating one or more symptoms associated with the disease.

The term “homogeneous” as used herein refers to something which is consistent or uniform in structure or composition throughout. In some examples, the term refers to cells having consistent maturation status, marker expression or phenotype within a given population.

As used herein, the term “inhibit” may refer to the act of blocking, reducing, eliminating, or otherwise antagonizing the presence, or an activity of, a particular target. For example, inhibiting the phosphorylation of Tau protein may refer to any act leading to decreasing, reducing, antagonizing eliminating, blocking or otherwise diminishing the phosphorylation of Tau protein. Inhibition may refer to partial inhibition or complete inhibition. In other examples, inhibition of the expression of a nucleic acid may include, but not limited to reduction in the transcription of a nucleic acid, reduction of mRNA abundance (e.g., silencing mRNA transcription), degradation of mRNA, inhibition of mRNA translation, and so forth.

As used herein, the term “suppress” may refer to the act of decreasing, reducing, prohibiting, limiting, lessening, or otherwise diminishing the presence, or an activity of, a particular target. Suppression may refer to partial suppression or complete suppression. For example, suppressing phosphorylation of Tau protein may refer to any act leading to decreasing, reducing, prohibiting, limiting, lessening, or otherwise diminishing the phosphorylation of Tau protein. In other examples, suppression of the expression of a nucleic acid may include, but not limited to reduction in the transcription of a nucleic acid, reduction of mRNA abundance (e.g., silencing mRNA transcription), degradation of mRNA, inhibition of mRNA translation, and so forth.

As used herein, the term “enhance” may refer to the act of improving, boosting, heightening, or otherwise increasing the presence, or an activity of, a particular target. For example, enhancing neuronal health may refer to any act leading to improving, boosting, heightening, or otherwise increasing neuronal health.

As used herein, the term “modulate” may refer to the act of changing, altering, varying, or otherwise modifying the presence, or an activity of, a particular target. For example, modulating a disease-associated component may include but not limited to any acts leading to changing, altering, varying, or otherwise modifying the amount of the disease-associated component. In some examples, “modulate” refers to enhancing the presence or activity of a particular target. In some examples, “modulate” refers to suppressing the presence or activity of a particular target. For example, modulating the amount of disease-associated component may include but is not limited to suppressing or enhancing the amount of the disease-associated component.

As used herein, the term “induce” may refer to the act of initiating, prompting, stimulating, establishing, or otherwise producing a result. For example, inducing an expression of mutant gene may refer to any act leading to initiating, prompting, stimulating, establishing, or otherwise producing the desired expression of the mutant gene. In other examples, inducing the expression of a nucleic acid may include, but not limited to initiation of the transcription of a nucleic acid, initiation of mRNA translation, and so forth.

As used herein “stem cell”, unless defined further, refers to any non-somatic cell. Any cell that is not a terminally differentiated or terminally committed cell may be referred to as a stem cell. This includes embryonic stem cells, induced pluripotent stem cells, hematopoietic stem cells, progenitor cells, and partially differentiated progenitor cells. Stem cells may be totipotent, pluripotent, or multipotent stem cells. Any cell which has the potential to differentiate into two different types of cells is considered a stem cell for the purpose of this application.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

For any of the structural and functional characteristics described herein, methods of determining these characteristics are known in the art.

### Derivation, Differentiation and Maturation of PSC-Derived Neurons

Human iPSCs have become powerful tools in modeling human diseases and hold tremendous potential for translational research in target discovery and drug development. Human iPSC derived neurons are sensitive and require extended culturing time (80 days) to develop mature neuron characteristics (Shi et al., 2012). Long term neuronal cell maintenance proves challenging using traditional manual techniques and thus, most small molecule and CRISPR screens were conducted using neurons cultured for less than 30 days (Boissart et al., 2013; Tian et al., 2019; Wang et al., 2017). Given that many neurodegenerative diseases are adult onset, such as Alzheimer's disease (AD), high throughput screening platforms combined with longer neuronal culture times could be more translationally relevant. With the development of modern automation technology and the increased use of human iPSC for disease modeling, an increased demand and implementation of automated culturing platforms for iPSC neurons is anticipated.

Alzheimer's disease (AD) is characterized by the pathological hallmarks of amyloid-O (AD) plaques, neurofibrillary tangles, astrogliosis, and neuronal loss. As plaques are composed of aggregated Aβ peptides, often surrounded by Phospho-Tau (pTau) positive dystrophic neurites (neuritic plaques) and activated microglia. Neurofibrillary tangles contain hyperphosphorylated Tau, with increased phosphorylation at several amino acid sites (Braak and Braak, 1991; Goedert et al., 2006; Petry et al., 2014; Spillantini and Goedert, 2013; Yu et al., 2009). Additional previously identified AD pathologies include cerebrovascular amyloid angiopathy, microgliosis, neuroinflammation, and major synaptic alteration (Crews and Masliah, 2010; Katzman, 1986; McGeer et al., 1988; Spillantini and Goedert, 2013).

The amyloid hypothesis proposes that abnormally folded Aβ peptides initiate a causal cascade beginning with Aβ oligomer aggregation into plaques, which then trigger Tau hyperphosphorylation and neurofibrillary tangle formation, ultimately resulting in neuronal cell death (De Strooper and Karran, 2016; Hardy and Selkoe, 2002). This hypothesis has been the theoretical foundation for the generation of numerous animal models, diagnostics, and drug development programs for AD (De Strooper and Karran, 2016). Supporting some aspects of this hypothesis, rodent AD models often overexpress mutated forms of the familial AD (FAD)-causing genes, APP and/or PSEN, leading to overproduction of Aβ peptides, extensive amyloid plaque formation, neuroinflammation, and some synaptic dysfunction (Ashe and Zahs, 2010; LaFerla and Green, 2012). However, important aspects of AD pathology such as p-Tau induction and severe neuronal loss have not been robustly established (Crews and Masliah, 2010; Kokjohn and Roher, 2009; Morrissette et al., 2009). The recent failures of many anti-Aβ therapeutics has cast some doubt on the amyloid hypothesis (Long and Holtzman, 2019; McDade and Bateman, 2017; von Schaper, 2018). Therefore, the relevance of existing rodent models for AD drug development is still debated (Ashe and Zahs, 2010; Morrissette et al., 2009; Sasaguri et al., 2017). Without robust animal, cellular, or translational models, the mechanisms by which Aβ oligomers trigger p-Tau induction and neuronal death have remained elusive; consequently, there are currently no disease modifying treatments for AD despite 40 years of intense research efforts.

For this reason, the development of improved model systems that more robustly mimic human AD pathophysiology is important for drug development and translation. Innovations in developing human induced pluripotent stem cell (iPSC) neuronal and microglial differentiation protocols have opened up new possibilities in translational models for human disease (Penney et al., 2020). Recent studies show that 3D cultures of human neurons overexpressing mutant APP in vitro led to pTau induction (Choi et al., 2014). Furthermore, implanting human iPSC neurons into AD mouse models recapitulates pTau induction and human neuronal sensitivity phenotypes not previously observed in traditional mouse models (Espuny-Camacho et al., 2017). While more translationally relevant, the techniques described above can be labor intensive and highly variable, and thus not ideal for drug screening and development.

Previous findings indicate that human neurons could be more translationally relevant to AD pathology. Disclosed herein is a human iPSC neuron culturing platform that is quantitative, high throughput, multiplexed, systematic, and reproducible to allow for pharmacological studies, mechanistic studies, and screening efforts. Also presented herein is a novel, high throughput human iPSC-based model of AD that recapitulates key hallmark pathologies that have been historically difficult to replicate in one model system. This model recapitulates robust Aβ plaque formation with surrounding pTau positive dystrophic neurites and human iPSC microglia for the first time in vitro. Consistent with AD pathologies, observed in the system are severe synapse loss, axon degeneration, and pTau induction resulting in severe neuronal loss. A focused compound library screen is also disclosed herein. We identified known kinase pathways—such as glycogen synthase kinase 3 (GSK3), Fyn, and dual leucine zipper kinase (DLK)—that have previously been implicated in AD, thereby validating the system as a useful screening tool. This platform is amenable for use to explore mechanisms of microglia-driven plaque formation. In addition, the model platform can also be used to investigate the mechanism of action (MOA) of anti-Aβ therapeutics and the findings highlight the importance of early administration and high exposure of therapeutic compounds. (Kaufman et al., 2015; Leclerc et al., 2001; Patel et al., 2015). In some aspects, disclosed herein is a robust platform that could facilitate target discovery, drug development, and impactful MOA studies in AD research towards a potential treatment.

### Automated Cell Culture System

Given that many neurodegenerative diseases are adult onset, such as Alzheimer's disease (AD), high throughput screening platforms combined with longer neuronal culture times could be more translationally relevant. In some aspects, the present invention provides an automated cell culture system for facilitating neuronal differentiation and/or promoting long-term neuronal growth, wherein the automated cell culture system comprises one or more rounds of automated culture media replacements. In some embodiments, the automated cell culture system sustains differentiation, maturation and/or growth of neuronal cells for at least about any one of 30, 60, 80, 90, 120, or 150 days.

In some embodiments, the automated cell culture system sustains differentiation, maturation and/or growth of neuronal cells for at least about any one of: 20, 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, or 200 days. In some embodiments, the automated cell culture system sustains differentiation, maturation and/or growth of neuronal cells for at least about any one of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the automated cell culture system sustains differentiation, maturation and/or growth of neuronal cells for at least about any one of: 55 to 60, 60 to 65, 65 to 70, 70 to 75, 75 to 80, 80 to 85, 85 to 90, or 90 to 100 days.

In some embodiments, the automated culture media replacement comprises automated culture media aspiration and automated culture media replenishment. In some embodiments, each round of automated culture media replacement comprises one or more rounds of automated culture media aspiration and one or more rounds of automated culture media replenishment. In some embodiments, the automated cell culture system comprises one or more tissue culture vessels. In some embodiments, the automated cell culture system comprises one or more tissue culture plates. In some embodiments, the automated cell culture system comprises one or more multi-well tissue culture plates. In some embodiments, the automated cell culture system comprises one or more 96-well tissue culture plates. In some embodiments, the automated cell culture system comprises one or more 384-well tissue culture plates.

### Automated Culture Media Aspiration

In some embodiments according to any of the embodiments described herein, the automated culture media aspiration comprises aspiration with a pipet tip. In some embodiments, the pipet tip comprises a distal end, wherein the distal end is a tapered end. In some embodiments, wherein the automated culture media aspiration comprises aspiration with a pipet tip, the distal end of the pipet tip is at about 1 mm above the bottom surface of the well before, during and/or after the aspiration. In some embodiments, the distal end of the pipet tip is at about any one of: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0 or 5.0 mm above the bottom surface of the well before the aspiration. In some embodiments, the distal end of the pipet tip is at about any one of: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0 or 5.0 mm above the bottom surface of the well during the aspiration. In some embodiments, the distal end of the pipet tip is at about any one of: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0 or 5.0 mm above the bottom surface of the well after the aspiration. In some embodiments, wherein the automated culture media aspiration comprises aspiration with a pipet tip, the distal end of the pipet tip is at any one of about: 0.1 to 0.2, 0.2 to 0.3, 0.3 to 0.4, 0.4 to 0.5, 0.5 to 0.6, 0.6 to 0.7, 0.7 to 0.8, 0.8 to 0.9, 0.9 to 1.0, 1.0 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 2.5, 2.5 to 3.0, or 3.0 to 5.0 mm above the bottom surface of the well before, during and/or after the aspiration.

In some embodiments, wherein the automated culture media aspiration comprises aspiration with a pipet tip, the pipet tip is at an angle of about 90° to the bottom surface of the well before, during and/or after the aspiration. In some embodiments, the pipet tip is at an angle of about any one of: 30°, 40°, 50°, 60°, 70°, 80°, or 90° before, during and/or after the aspiration. In some embodiments, the pipet tip is at an angle of about any one of: 70°, 72°, 74°, 76°, 78°, 80°, 82°, 84°, 86°, 88°, 90° before, during and/or after the aspiration. In some embodiments, wherein the automated culture media aspiration comprises aspiration with a pipet tip, the pipet tip is at an angle of any one of about: 30° to 40°, 40° to 50°, 50° to 60°, 60° to 70°, 70° to 80°, or 80° to 90° before, during and/or after the aspiration. In some embodiments, the pipet tip is at an angle of any one of about: 70° to 75°, 75° to 80°, 80° to 82°, 82° to 84°, 84° to 86°, 86° to 88°, or 88° to 90° before, during and/or after the aspiration.

In some embodiments, wherein the automated culture media aspiration comprises aspiration with a pipet tip, the pipet tip has a displacement of no more than about 0.1 mm from the center of the well before, during and/or after the aspiration. In some embodiments, the pipet tip has a displacement of no more than about any one of: 0.01, 0.02, 0.03, 0.04, 0.05, 0.06, 0.07, 0.08, 0.09, 0.1, 0.11, 0.12, 0.13, 0.14, 0.15 or 0.2 mm from the center of the well before, during and/or after the aspiration. In some embodiments, the pipet tip has a displacement of no more than about any one of: 0.01, 0.02, 0.03, 0.04, 0.05, 0.06, 0.07, 0.08, 0.09, 0.1, 0.11, 0.12, 0.13, 0.14, 0.15 or 0.2 mm from the center of the well before, during and/or after the aspiration. In some embodiments, the pipet tip is at the center of the well before, during and/or after the aspiration (no displacement).

In some embodiments, wherein the automated culture media aspiration comprises aspiration with a pipet tip, the speed of media aspiration is no more than about 7.5 μl/s. In some embodiments, the speed of media aspiration is no more than about any one of: 0.5, 1, 2, 3, 4, 5, 6, 7, 7.5, 8, 9, 10, 12, 15, 20, 25 or 30 μl/s. In some embodiments, the speed of media aspiration is no more than any one of about: 0.5 to 1, 1 to 2, 2 to 3, 3 to 4, 4 to 5, 5 to 6, 6 to 7, 7 to 8, 8 to 9, 9 to 10, 10 to 12, 12 to 15, 15 to 20, 20 to 25, or 25 to 30 μl/s. In some embodiments, the start of media aspiration is about 200 ms subsequent to the pipet tip being placed 1 mm above the bottom surface of the well. In some embodiments, the start of media aspiration is about any one of: 5, 10, 20, 50, 80, 100, 150, 200, 250, 300, 350, 400, 450, 500, 600, 700, 800, 900 or 1000 ms subsequent to the pipet tip being placed x mm above the bottom surface of the well, wherein x is any one of about: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0 or 5.0. In some embodiments, the start of media aspiration is any one of about: 5 to 10, 10 to 20, 20 to 50, 50 to 80, 80 to 100, 100 to 150, 150 to 200, 200 to 250, 250 to 300, 300 to 350, 350 to 400, 400 to 450, 450 to 500, 500 to 600, 600 to 700, 700 to 800, 800 to 900 or 900 to 1000 ms subsequent to the pipet tip being placed x mm above the bottom surface of the well, wherein x is any one of about: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0 or 5.0.

In some embodiments, wherein the automated culture media aspiration comprises aspiration with a pipet tip, the pipet tip is inserted into the well at a speed of about 5 mm/s prior to aspiration. In some embodiments, the pipet tip is inserted into the well at a speed of about any one of: 0.5, 1, 2, 3, 4, 5, 6, 7, 8, 9, 10, 12, 15, 20, 25 or 30 mm/s prior to aspiration. In some embodiments, the pipet tip is inserted into the well at a speed of any one of about: 0.5 to 1, 1 to 2, 2 to 3, 3 to 4, 4 to 5, 5 to 6, 6 to 7, 7 to 8, 8 to 9, 9 to 10, 10 to 12, 12 to 15, 15 to 20, 20 to 25, or 25 to 30 mm/s prior to aspiration.

In some embodiments, wherein the automated culture media aspiration comprises aspiration with a pipet tip, the pipet tip is withdrawn from the well at a speed of about 5 mm/s after the aspiration. In some embodiments, the pipet tip is withdrawn from the well at a speed of about any one of: 0.5, 1, 2, 3, 4, 5, 6, 7, 8, 9, 10, 12, 15, 20, 25 or 30 mm/s after the aspiration. In some embodiments, the pipet tip is withdrawn from the well at a speed of any one of about: 0.5 to, 11 to 2, 2 to 3, 3 to 4, 4 to 5, 5 to 6, 6 to 7, 7 to 8, 8 to 9, 9 to 10, 10 to 12, 12 to 15, 15 to 20, 20 to 25, or 25 to 30 mm/s after the aspiration.

In some embodiments, wherein the cell culture system comprises an N-well plate; the automated cell culture system comprises automated discarding of a used rack of N-pipet tips and automated engagement of a new rack of N-pipet tips subsequent to each round of media aspiration, wherein Nis an integer of 6, 12, 24, 48, 96, 182 or 384. In some embodiments, wherein the cell culture system comprises a 384-well plate; the automated cell culture system comprises automated discarding of a used rack of 384-pipet tips and automated engagement of a new rack of 384-pipet tips subsequent to each round of media aspiration.

In some embodiments, wherein the cell culture system comprises one or more batches of N-well plates, wherein each batch comprises a plurality of N-well plates arranged my columns and z rows; the automated cell culture system comprises automated discarding of up to (y multiplied by z) corresponding used racks of N-pipet tips and automated engagement of up to (y multiplied by z) corresponding new racks of N-pipet tips subsequent to each round of media aspiration, wherein N is an integer of 6, 12, 24, 48, 96, 182 or 384, wherein y is an integer of 1, 2, 3, 4, 5, 6, 7, 8, 9, 10, 12, 13, 14, 15, 16, 17, 18, 19, 20 and wherein z is an integer of 1, 2, 3, 4, 5, 6, 7, 8, 9, 10, 12, 13, 14, 15, 16, 17, 18, 19, 20. In some embodiments, wherein the cell culture system comprises one or more batches of 384-well plates, wherein each batch comprises up to twenty-five 384-well plates arranged in 5 columns and 5 rows; the automated cell culture system comprises automated discarding of up to 25 corresponding used racks of 384-pipet tips and automated engagement of up to 25 corresponding new racks of 384-pipet tips subsequent to each round of media aspiration.

### Automated Culture Media Dispensing

In some embodiments according to any of the embodiments described herein, the automated culture media replenishment comprises dispensing media with a pipet tip. In some embodiments, the pipet tip comprises a distal end, wherein the distal end is a tapered end. In some embodiments, wherein the automated culture media replenishment comprises dispensing media with a pipet tip, the distal end of the pipet tip is at about 1 mm above the bottom surface of the well before, during and/or after the dispensing. In some embodiments, the distal end of the pipet tip is at about any one of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0 or 5.0 mm above the bottom surface of the well before the dispensing. In some embodiments, the distal end of the pipet tip is at about any one of: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 4.0, 5.0, 6.0, 7.0, 8.0, 9.0, 10.0, 11.0, 12.0, 12.4, 13, 14, 15, 16, 17, 18, 19 or 20 mm above the bottom surface of the well during the dispensing. In some embodiments, the distal end of the pipet tip is at about any one of: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 4.0, 5.0, 6.0, 7.0, 8.0, 9.0, 10.0, 11.0, 12.0, 12.4, 13, 14, 15, 16, 17, 18, 19 or 20 mm above the bottom surface of the well after the dispensing. In some embodiments, wherein the automated culture media replenishment comprises dispensing media with a pipet tip, the distal end of the pipet tip is at any one of about: 0.1 to 0.2, 0.2 to 0.3, 0.3 to 0.4, 0.4 to 0.5, 0.5 to 0.6, 0.6 to 0.7, 0.7 to 0.8, 0.8 to 0.9, 0.9 to 1.0, 1.0 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 2.5, 2.5 to 3.0, or 3.0 to 5.0 mm above the bottom surface of the well before, during and/or after the dispensing.

In some embodiments, wherein the automated culture media replenishment comprises dispensing media with a pipet tip, the pipet tip is withdrawn from the well at a speed of about 1 mm/s during the dispensing. In some embodiments, the pipet tip is withdrawn from the well at a speed of about any one of: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0 or 5.0 mm/s during the dispensing. In some embodiments, wherein the automated culture media replenishment comprises dispensing media with a pipet tip, the pipet tip is withdrawn from the well at a speed of any one of about: 0.1 to 0.2, 0.2 to 0.3, 0.3 to 0.4, 0.4 to 0.5, 0.5 to 0.6, 0.6 to 0.7, 0.7 to 0.8, 0.8 to 0.9, 0.9 to 1.0, 1.0 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 2.5, 2.5 to 3.0, or 3.0 to 5.0 mm/s during the dispensing.

In some embodiments, wherein the automated culture media replenishment comprises dispensing media with a pipet tip, the pipet tip is at an angle of about 90° to the bottom surface of the well before, during and/or after the dispensing. In some embodiments, the pipet tip is at an angle of about any one of: 30°, 40°, 50°, 60°, 70°, 80°, or 90° before, during and/or after the dispensing. In some embodiments, the pipet tip is at an angle of about any one of: 70°, 72°, 74°, 76°, 78°, 80°, 82°, 84°, 86°, 88°, 90° before, during and/or after the dispensing. In some embodiments, wherein the automated culture media replenishment comprises dispensing media with a pipet tip, the pipet tip is at an angle of any one of about: 30° to 40°, 40° to 50°, 50° to 60°, 60° to 70°, 70° to 80°, or 80° to 90° before, during and/or after the dispensing. In some embodiments, the pipet tip is at an angle of any one of about: 70° to 75°, 75° to 80°, 80° to 82°, 82° to 84°, 84° to 86°, 86° to 88°, or 88° to 90° before, during and/or after the dispensing.

In some embodiments, wherein the automated culture media replenishment comprises dispensing media with a pipet tip, the pipet tip has a displacement of no more than about 0.1 mm from the center of the well before, during and/or after the dispensing. In some embodiments, the pipet tip has a displacement of no more than about any one of: 0.01, 0.02, 0.03, 0.04, 0.05, 0.06, 0.07, 0.08, 0.09, 0.1, 0.11, 0.12, 0.13, 0.14, 0.15 or 0.2 mm from the center of the well before, during and/or after the dispensing. In some embodiments, the pipet tip has a displacement of no more than about any one of: 0.01, 0.02, 0.03, 0.04, 0.05, 0.06, 0.07, 0.08, 0.09, 0.1, 0.11, 0.12, 0.13, 0.14, 0.15 or 0.2 mm from the center of the well before, during and/or after the dispensing. In some embodiments, the pipet tip is at the center of the well before, during and/or after the dispensing (no displacement).

In some embodiments, wherein the automated culture media replenishment comprises dispensing media with a pipet tip, the pipet tip is displaced (such as laterally displaced) to contact a first side of the well about any one of: 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 4.0, 4.5 or 5.0 mm from the center in a first direction, at a height of about any one of: 2.0, 3.0, 4.0, 5.0, 6.0, 7.0, 8.0, 9.0, 10.0, 11.0, 12.0, 12.4, 13, 14, 15, 16, 17, 18, 19 or 20 mm above the bottom of the well at a speed of about any one of: 20, 50, 80, 100, 150, 200, 250, 300, 350, 400, 450, or 500 mm/s. In some embodiments, the pipet tip is displaced (such as laterally displaced) to contact a first side of the well 1 mm from the center in a first direction, at a height of about 12.40 mm above the bottom of the well at a speed of about 100 mm/s. In some embodiments, the pipet tip is displaced (such as laterally displaced) to contact a second side of the well about any one of: 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 4.0, 4.5 or 5.0 mm from the center in a second direction, at a height of about any one of: 2.0, 3.0, 4.0, 5.0, 6.0, 7.0, 8.0, 9.0, 10.0, 11.0, 12.0, 12.4, 13, 14, 15, 16, 17, 18, 19 or 20 mm above the bottom of the well at a speed of about any one of: 20, 50, 80, 100, 150, 200, 250, 300, 350, 400, 450, or 500 mm/s. In some embodiments, the pipet tip is displaced (such as laterally displaced) to contact a second side of the well 1 mm from the center in a second direction, at a height of about 12.40 mm above the bottom of the well at a speed of about 100 mm/s. In some embodiments, the first direction is at an angle of about any one of: 30°, 40°, 50°, 60°, 70°, 80°, 90°, 100°, 110°, 120°, 130°, 140°, 150°, 160°, 170°, 180°, 190°, 200°, 210°, 220°, 230°, 240°, 250°, 260°, 270°, 280°, 290°, 300°, 310°, 320°, 330°, (or any angle there between) to the second direction. In some embodiments, the first direction is at an angle of about 180° to the second direction.

In some embodiments, wherein the automated culture media replenishment comprises dispensing media with a pipet tip, the speed of media dispensing is no more than about 1.5 μl/s. In some embodiments, the speed of media dispensing is no more than about any one of: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 5.0, 7.5 or 10.0 μl/s. In some embodiments, the speed of media dispensing is no more than any one of about: 0.1 to 0.2, 0.2 to 0.3, 0.3 to 0.4, 0.4 to 0.5, 0.5 to 0.6, 0.6 to 0.7, 0.7 to 0.8, 0.8 to 0.9, 0.9 to 1.0, 1.0 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 2.5, 2.5 to 3.0, 3.0 to 5.0, 5.0 to 7.5, or 7.5 to 10.0 μl/s. In some embodiments, the acceleration of media dispensing is about any one of: 20, 50, 100, 200, 300, 400, 500, 600, 700, 800, 900, 1000, 2000, 5000 μl/s² or any value in between, optionally wherein the acceleration of media dispensing occurs at the start of dispensing. In some embodiments, the deceleration of media dispensing is about 20, 50, 100, 200, 300, 400, 500, 600, 700, 800, 900, 1000, 2000, 5000 μl/s² or any value in between, optionally wherein the deceleration of media dispensing occurs at the end of dispensing. In some embodiments, the acceleration of media dispensing is about 500 μl/s², optionally wherein the acceleration of media dispensing occurs at the start of dispensing. In some embodiments, the deceleration of media dispensing is about 500 μl/s², optionally wherein the deceleration of media dispensing occurs at the end of dispensing.

In some embodiments, the start of media dispensing is about 200 ms subsequent to the pipet tip being placed 1 mm above the bottom surface of the well. In some embodiments, the start of media dispensing is about any one of: 5, 10, 20, 50, 80, 100, 150, 200, 250, 300, 350, 400, 450, 500, 600, 700, 800, 900 or 1000 ms subsequent to the pipet tip being placed x mm above the bottom surface of the well, wherein x is any one of about: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0 or 5.0. In some embodiments, the start of media dispensing is any one of about: 5 to 10, 10 to 20, 20 to 50, 50 to 80, 80 to 100, 100 to 150, 150 to 200, 200 to 250, 250 to 300, 300 to 350, 350 to 400, 400 to 450, 450 to 500, 500 to 600, 600 to 700, 700 to 800, 800 to 900 or 900 to 1000 ms subsequent to the pipet tip being placed x mm above the bottom surface of the well, wherein x is any one of about: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0 or 5.0.

In some embodiments, wherein the automated culture media replenishment comprises dispensing media with a pipet tip, the pipet tip is inserted into the well at a speed of about 5 mm/s prior to dispensing. In some embodiments, the pipet tip is inserted into the well at a speed of about any one of: 0.5, 1, 2, 3, 4, 5, 6, 7, 8, 9, 10, 12, 15, 20, 25 or 30 mm/s prior to dispensing. In some embodiments, the pipet tip is inserted into the well at a speed of any one of about: 0.5 to 1, 1 to 2, 2 to 3, 3 to 4, 4 to 5, 5 to 6, 6 to 7, 7 to 8, 8 to 9, 9 to 10, 10 to 12, 12 to 15, 15 to 20, 20 to 25, or 25 to 30 mm/s prior to dispensing.

In some embodiments, wherein the automated culture media replenishment comprises dispensing media with a pipet tip, the pipet tip is withdrawn from the well at a speed of about 5 mm/s after the dispensing. In some embodiments, the pipet tip is withdrawn from the well at a speed of about any one of: 0.5, 1, 2, 3, 4, 5, 6, 7, 8, 9, 10, 12, 15, 20, 25 or 30 mm/s after the dispensing. In some embodiments, the pipet tip is withdrawn from the well at a speed of any one of about: 0.5 to 1, 1 to 2, 2 to 3, 3 to 4, 4 to 5, 5 to 6, 6 to 7, 7 to 8, 8 to 9, 9 to 10, 10 to 12, 12 to 15, 15 to 20, 20 to 25, or 25 to 30 mm/s after the dispensing.

In some embodiments, wherein the cell culture system comprises an N-well plate; the automated cell culture system comprises automated discarding of a used rack of N-pipet tips and automated engagement of a new rack of N-pipet tips subsequent to each round of media dispensing, wherein N is an integer of 6, 12, 24, 48, 96, 182 or 384. In some embodiments, wherein the cell culture system comprises a 384-well plate; the automated cell culture system comprises automated discarding of a used rack of 384-pipet tips and automated engagement of a new rack of 384-pipet tips subsequent to each round of media dispensing.

In some embodiments, wherein the cell culture system comprises one or more batches of N-well plates, wherein each batch comprises a plurality of N-well plates arranged in y columns and z rows; the automated cell culture system comprises automated discarding of up to (y multiplied by z) corresponding used racks of N-pipet tips and automated engagement of up to (y multiplied by z) corresponding new racks of N-pipet tips subsequent to each round of media dispensing, wherein Nis an integer of 6, 12, 24, 48, 96, 182 or 384, wherein y is an integer of 1, 2, 3, 4, 5, 6, 7, 8, 9, 10, 12, 13, 14, 15, 16, 17, 18, 19, 20 and wherein z is an integer of 1, 2, 3, 4, 5, 6, 7, 8, 9, 10, 12, 13, 14, 15, 16, 17, 18, 19, 20. In some embodiments, wherein the cell culture system comprises one or more batches of 384-well plates, wherein each batch comprises up to twenty-five 384-well plates arranged in 5 columns and 5 rows; the automated cell culture system comprises automated discarding of up to 25 corresponding used racks of 384-pipet tips and automated engagement of up to 25 corresponding new racks of 384-pipet tips subsequent to each round of media dispensing.

In some embodiments according to any one of the automated cell culture system described herein, the system comprises about any one of 1, 2, 3, 4, 5, 6, 7, 8, 9, 10, 12, 15, 18, 20 or 25 rounds of automated culture media replacements. In some embodiments, the time interval between two rounds of culture media replacements is about any one of: 1, 2, 3, 4, 5, 6, 7, 8, 9, or 10 days. In some embodiments, the time interval between two successive rounds of culture media replacements is about any one of: 1, 2, 3, 4, 5, 6, 7, 8, 9, or 10 days. In some embodiments, the time interval between two rounds of culture media replacements is about 3 or 4 days. In some embodiments, the time interval between two successive rounds of culture media replacements is about 3 or 4 days.

In some embodiments according to any one of the automated cell culture system described herein, about any one of: 30%, 40%, 50%, 60%, 70%, or 80% of culture media is replaced in one or more rounds of culture media replacement. In some embodiments, about any one of: 40%, 42%, 44%, 46%, 48%, 50%, 52%, 54%, 56%, 58%, or 60% of culture media is replaced in one or more rounds of culture media replacement. In some embodiments, any one of about: 30% to 40%, 40% to 50%, 50% to 60%, 60% to 70%, or 70% to 80% of culture media is replaced in one or more rounds of culture media replacement. In some embodiments, about 50% of culture media is replaced in one or more rounds of culture media replacement.

In some embodiments according to any one of the automated cell culture system described herein, about any one of: 30%, 40%, 50%, 60%, 70%, or 80% of culture media is replaced in each round of culture media replacement. In some embodiments, about any one of: 40%, 42%, 44%, 46%, 48%, 50%, 52%, 54%, 56%, 58%, or 60% of culture media is replaced in each round of culture media replacement. In some embodiments, any one of about: 30% to 40%, 40% to 50%, 50% to 60%, 60% to 70%, or 70% to 80% of culture media is replaced in each round of culture media replacement. In some embodiments, about 50% of culture media is replaced in each round of culture media replacement.

### Methods of Generating Fully Mature PSC-Derived Neurons

In some aspects, the present invention provides a method of generating homogenous and/or terminally differentiated neurons from precursor cells. In some embodiments, there is provided a method of a method of generating homogenous and/or terminally differentiated neurons from neural stem cells (NSCs). In some embodiments, the method comprises: (a) differentiating NSCs into NSC-derived neurons; (b) replating the NSC-derived neurons in presence of primary human astrocytes; (c) differentiating and maturing the PSC-derived neurons for at least about 60 to about 90 days in an automated cell culture system. In some embodiments, the method comprises: (a) culturing the NSCs under conditions to increase the levels of NGN2 and ASCL1, in combination with a cell cycle inhibitor for at least about 7 days, thereby generating NSC-derived neurons; (b) replating the NSC-derived neurons in presence of primary human astrocytes; (c) differentiating and maturing the NSC-derived neurons for at least about 60 to about 90 days in an automated cell culture system.

In some embodiments, provided is a method of generating homogenous and/or terminally differentiated neurons from pluripotent stem cells (PSCs). In some embodiments, the method of generating homogenous and/or terminally differentiated neurons from pluripotent stem cells (PSCs) comprises: (a) generating a pluripotent stem cell- (PSC-) derived neural stem cell (NSC) line expressing NGN2, and ASCL1 under an inducible system; (b) culturing the NSC line under conditions to induce the expression of NGN2 and ASCL1, in combination with a cell cycle inhibitor for at least about 7 days, thereby generating PSC-derived neurons; (c) replating the PSC-derived neurons in presence of primary human astrocytes; and/or (d) differentiating and/or maturing the PSC-derived neurons for at least about 60 to about 90 days in an automated cell culture system.

In some embodiments, the step of differentiating and/or maturing the PSC-derived neurons comprises differentiating and/or maturing the PSC-derived neurons in any one of the automated cell culture systems described above. In some embodiments, the step of differentiating and/or maturing the NSC-derived neurons comprises differentiating and/or maturing the NSC-derived neurons in any one of the automated cell culture systems described above.

In some embodiments, the step of differentiating and/or maturing the PSC-derived neurons comprises one or more rounds of automated culture media replacements using an automated cell culture system; and wherein the automated cell culture system sustains differentiation, maturation and/or growth of neuronal cells for at least about any one of: 20, 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, or 200 days. In some embodiments, the step of differentiating and/or maturing the PSC-derived neurons comprises one or more rounds of automated culture media replacements using an automated cell culture system; and wherein the automated cell culture system sustains differentiation, maturation and/or growth of neuronal cells for at least about any one of: 30, 60, 80, 90, 120, or 150 days. In some embodiments, the step of differentiating and/or maturing the PSC-derived neurons comprises one or more rounds of automated culture media replacements using an automated cell culture system; and wherein the automated cell culture system sustains differentiation, maturation and/or growth of neuronal cells for at least about 60 days.

In some embodiments, the automated culture media replacement comprises automated culture media aspiration and automated culture media replenishment. In some embodiments, the automated cell culture system comprises one or more tissue culture plates. In some embodiments, the automated cell culture system comprises one or more multi-well tissue culture plates. In some embodiments, the automated cell culture system comprises one or more 96-well tissue culture plates. In some embodiments, the automated cell culture system comprises one or more 384-well tissue culture plates.

In some embodiments according to any one of the methods described herein, the automated culture media aspiration comprises aspiration with a pipet tip, further wherein: (a) the distal end of the pipet tip is at about 0.8 mm to about 1.2 mm above the bottom surface of the well before, during and/or after the aspiration; (b) the pipet tip is at an angle of about 800 to about 90° to the bottom surface of the well before, during and/or after the aspiration; (c) the pipet tip has a displacement of no more than 0.2 mm from the center of the well before, during and/or after the aspiration; optionally wherein the pipet tip is at the center of the well before, during and/or after the aspiration (no displacement); (e) the speed of media aspiration is no more than about 15 μl/s; (f) the start of media aspiration is about 100 ms to about 500 ms subsequent to the pipet tip being placed 1 mm above the bottom surface of the well; (g) the pipet tip is inserted into the well at a speed of about 1 mm/s to about 10 mm/s prior to aspiration; and/or (h) the pipet tip is withdrawn from the well at a speed of about 1 mm/s to about 10 mm/s after aspiration.

In some embodiments according to any one of the methods described herein, the automated culture media aspiration comprises aspiration with a pipet tip, further wherein: (a) the distal end of the pipet tip is at about 1 mm above the bottom surface of the well before, during and/or after the aspiration; (b) the pipet tip is at an angle of about 90° to the bottom surface of the well before, during and/or after the aspiration; (c) the pipet tip has a displacement of no more than 0.1 mm from the center of the well before, during and/or after the aspiration; optionally wherein the pipet tip is at the center of the well before, during and/or after the aspiration (no displacement); (e) the speed of media aspiration is no more than about 7.5 μl/s; (f) the start of media aspiration is about 200 ms subsequent to the pipet tip being placed 1 mm above the bottom surface of the well; (g) the pipet tip is inserted into the well at a speed of about 5 mm/s prior to aspiration; and/or (h) the pipet tip is withdrawn from the well at a speed of about 5 mm/s after aspiration.

In some embodiments according to any one of the methods described herein, the automated culture media replenishment comprises dispensing media with a pipet tip, further wherein: (a) the distal end of the pipet tip is at about 0.8 mm to about 1.2 mm above the bottom surface of the well before the dispensing; (b) the distal end of the pipet tip is withdrawn from the well at about 1 mm/s during the dispensing; (c) the pipet tip is at an angle of about 800 to about 90° to the bottom surface of the well before and/or during the dispensing; (d) the pipet tip has a displacement of no more than 0.2 mm from the center of the well before, and/or during the dispensing, optionally wherein the pipet tip is at the center of the well before, and/or during the dispensing (no displacement); (e) the pipet tip is displaced (such as displaced laterally) to contact a first side of the well about 0.8 mm to about 1.2 mm from the center in a first direction, at a height of about 10 mm to about 15 mm above the bottom of the well at a speed of about 50 mm/s to about 200 mm/s; (f) the pipet tip is displaced (such as displaced laterally) to contact a second side of the well about 0.8 mm to about 1.2 mm from the center in a second direction, at a height of about 10 mm to about 15 mm above the bottom of the well at a speed of about 50 mm/s to about 200 mm/s, optionally wherein the first direction is at an angle of about 1600 to about 2000 to the second direction; (g) the speed of media dispensing is no more than about 5 μl/s; (h) the acceleration of media dispensing is about 200 μl/s² to about 1000 μl/s²; (i) the deceleration of media dispensing is about 200 μl/s² to about 1000 μl/s²; (j) the start of media dispensing is about 100 ms to about 500 ms subsequent to the pipet tip being placed 1 mm above the bottom surface of the well; (k) the pipet tip is inserted into the well at a speed of about 1 mm/s to about 10 mm/s prior to dispensing; and/or (l) the pipet tip is withdrawn from the well at a speed of about 1 mm/s to about 10 mm/s after dispensing. In some embodiments, the pipet tip is displaced (such as displaced laterally) before, during and/or after the dispensing. In some embodiments, the pipet tip is displaced laterally during the dispensing. In some embodiments, the pipet tip is displaced laterally after the dispensing. In some embodiments, the pipet tip is displaced laterally before and/or during being withdrawn from the well.

In some embodiments according to any one of the methods described herein, the automated culture media replenishment comprises dispensing media with a pipet tip, further wherein: (a) the distal end of the pipet tip is at about 1 mm above the bottom surface of the well before the dispensing; (b) the distal end of the pipet tip is withdrawn from the well at about 1 mm/s during the dispensing; (c) the pipet tip is at an angle of about 90° to the bottom surface of the well before and/or during the dispensing; (d) the pipet tip has a displacement of no more than 0.1 mm from the center of the well before, and/or during the dispensing, optionally wherein the pipet tip is at the center of the well before, and/or during the dispensing (no displacement); (e) the pipet tip is displaced (such as displaced laterally) to contact a first side of the well about 1 mm from the center in a first direction, at a height of about 12.40 mm above the bottom of the well at a speed of about 100 mm/s; (f) the pipet tip is displaced (such as displaced laterally) to contact a second side of the well about 1 mm from the center in a second direction, at a height of about 12.40 mm above the bottom of the well at a speed of about 100 mm/s, optionally wherein the first direction is at an angle of about 1800 to the second direction; (g) the speed of media dispensing is no more than about 1.5 μl/s; (h) the acceleration of media dispensing is about 500 μl/s²; (i) the deceleration of media dispensing is about 500 μl/s²; (j) the start of media dispensing is about 200 ms subsequent to the pipet tip being placed 1 mm above the bottom surface of the well; (k) the pipet tip is inserted into the well at a speed of about 5 mm/s prior to dispensing; and/or (l) the pipet tip is withdrawn from the well at a speed of about 5 mm/s after dispensing. In some embodiments, the pipet tip is displaced (such as displaced laterally) before, during and/or after the dispensing. In some embodiments, the pipet tip is displaced laterally during the dispensing. In some embodiments, the pipet tip is displaced laterally after the dispensing. In some embodiments, the pipet tip is displaced laterally before and/or during being withdrawn from the well.

In some embodiments, wherein the cell culture system comprises one or more batches of 384-well plates, wherein each batch comprises up to twenty-five 384-well plates arranged in 5 columns and 5 rows; the automated cell culture system comprises automated discarding of up to 25 corresponding used racks of 384-pipet tips and automated engagement of up to 25 corresponding new racks of 384-pipet tips subsequent to each round of media aspiration. In some embodiments, wherein the cell culture system comprises one or more batches of 384-well plates, wherein each batch comprises up to twenty-five 384-well plates arranged in 5 columns and 5 rows; the automated cell culture system comprises automated discarding of up to 25 corresponding used racks of 384-pipet tips and automated engagement of up to 25 corresponding new racks of 384-pipet tips subsequent to each round of media dispensing.

In some embodiments according to any one of the methods described herein, the method comprises about any one of 1, 2, 3, 4, 5, 6, 7, 8, 9, 10, 12, 15, 18, 20 or 25 rounds of automated culture media replacements. In some embodiments, the time interval between two rounds of culture media replacements is about any one of: 1, 2, 3, 4, 5, 6, 7, 8, 9, or 10 days. In some embodiments, the time interval between two successive rounds of culture media replacements is about any one of: 1, 2, 3, 4, 5, 6, 7, 8, 9, or 10 days. In some embodiments, the time interval between two rounds of culture media replacements is about 3 or 4 days. In some embodiments, the time interval between two successive rounds of culture media replacements is about 3 or 4 days.

In some embodiments according to any one of the methods described herein, about any one of: 30%, 40%, 50%, 60%, 70%, or 80% of culture media is replaced in one or more rounds of culture media replacement. In some embodiments, about any one of: 40%, 42%, 44%, 46%, 48%, 50%, 52%, 54%, 56%, 58%, or 60% of culture media is replaced in one or more rounds of culture media replacement. In some embodiments, any one of about: 30% to 40%, 40% to 50%, 50% to 60%, 60% to 70%, or 70% to 80% of culture media is replaced in one or more rounds of culture media replacement. In some embodiments, about 50% of culture media is replaced in one or more rounds of culture media replacement.

In some embodiments according to any one of the methods described herein, about any one of: 30%, 40%, 50%, 60%, 70%, or 80% of culture media is replaced in each round of culture media replacement. In some embodiments, about any one of: 40%, 42%, 44%, 46%, 48%, 50%, 52%, 54%, 56%, 58%, or 60% of culture media is replaced in each round of culture media replacement. In some embodiments, any one of about: 30% to 40%, 40% to 50%, 50% to 60%, 60% to 70%, or 70% to 80% of culture media is replaced in each round of culture media replacement. In some embodiments, about 50% of culture media is replaced in each round of culture media replacement.

The use of any one of the methods described herein for deriving differentiated neurons in a system for modeling neurodegenerative diseases, wherein the system comprises substantially defined culture media and wherein the system is amenable to modular and tunable inputs of: one or more disease-associated components and/or one or more neuroprotective components.

### Fully Mature PSC-Derived Neurons

In some aspects, the present invention provides a homogenous population of terminally differentiated neurons derived from precursor cells. In some embodiments, there is provided a homogenous population of terminally differentiated neurons derived from neural stem cells (NSCs).

In some embodiments, provided is a homogenous population of terminally differentiated neurons derived from pluripotent stem cells, wherein at least about any one of: 50%, 55%, 60%, 65%, 70%, 75%, 80%, 85%, 90%, 91%, 92%, 93%, 94%, 95%, 96%, 97%, or 98% of the neurons express: Map2; Synapsin 1 and/or Synapsin 2; and beta-III tubulin. In some embodiments, provided is a homogenous population of terminally differentiated neurons derived from pluripotent stem cells, wherein at least about 95% of the neurons express: Map2; Synapsin 1 and/or Synapsin 2; and beta-III tubulin. In some embodiments, at least about any one of: 80%, 85%, 90%, 91%, 92%, 93%, 94%, 95%, 96%, 97%, or 98% of the neurons express Map2. In some embodiments, at least about any one of: 80%, 85%, 90%, 91%, 92%, 93%, 94%, 95%, 96%, 97%, or 98% of the neurons express Synapsin 1 and/or Synapsin 2. In some embodiments, at least about any one of: 80%, 85%, 90%, 91%, 92%, 93%, 94%, 95%, 96%, 97%, or 98% of the neurons express beta-III tubulin.

In some embodiments, provided is a homogenous population of terminally differentiated neurons derived from pluripotent stem cells, wherein at least about 80% of the terminally differentiated neurons express Map2 at a level that is at least about any one of: 20%, 50%, 80%, 100%, 2-fold, 3-fold, 5-fold, 10-fold, 20-fold, 50-fold, 100-fold, 500-fold, 1000-fold, 10000-fold, 100000-fold higher than a non-terminally differentiated neuron. In some embodiments, provided is a homogenous population of terminally differentiated neurons derived from pluripotent stem cells, wherein at least about 80% of the terminally differentiated neurons express Synapsin 1 and/or Synapsin 2 at a level that is at least about any one of: 20%, 50%, 80%, 100%, 2-fold, 3-fold, 5-fold, 10-fold, 20-fold, 50-fold, 100-fold, 500-fold, 1000-fold, 10000-fold, 100000-fold higher than a non-terminally differentiated neuron. In some embodiments, provided is a homogenous population of terminally differentiated neurons derived from pluripotent stem cells, wherein at least about 80% of the terminally differentiated neurons express beta-III tubulin at a level that is at least about any one of: 20%, 50%, 80%, 100%, 2-fold, 3-fold, 5-fold, 10-fold, 20-fold, 50-fold, 100-fold, 500-fold, 1000-fold, 10000-fold, 100000-fold higher than a non-terminally differentiated neuron.

In some embodiments, provided is a homogenous population of terminally differentiated neurons derived from pluripotent stem cells, wherein at least about any one of: 50%, 55%, 60%, 65%, 70%, 75%, 80%, 85%, 90%, 91%, 92%, 93%, 94%, 95%, 96%, 97%, or 98% of the neurons express one or more pre-synaptic markers selected from vGLUT2, Synapsin 1, and Synapsin 2. In some embodiments, provided is a homogenous population of terminally differentiated neurons derived from pluripotent stem cells, wherein at least about 95% of the neurons express one or more pre-synaptic markers selected from vGLUT2, Synapsin 1, and Synapsin 2. In some embodiments, at least about any one of: 50%, 55%, 60%, 65%, 70%, 75%, 80%, 85%, 90%, 91%, 92%, 93%, 94%, 95%, 96%, 97%, or 98% of the neurons express one or more post-synaptic markers selected from: PSD95, SHANK, PanSHANK, GluR1, GluR2, PanSAPAP, and NR1. In some embodiments, provided is a homogenous population of terminally differentiated neurons derived from pluripotent stem cells, wherein at least about 95% of the neurons express one or more post-synaptic markers selected from: PSD95, SHANK, PanSHANK, GluR1, GluR2, PanSAPAP, and NR1. In some embodiments, at least about any one of: 20, 30, 50, 80, 100, 200, 300, 500, 800, or 1000 postsynaptic endings of a neuron overlap with presynaptic endings of other neurons and/or at least about any one of: 20, 30, 50, 80, 100, 200, 300, 500, 800, or 1000 presynaptic endings of the neuron overlap with postsynaptic endings of other neurons. In some embodiments, at least 100 postsynaptic endings of a neuron overlap with presynaptic endings of other neurons and/or at least 100 presynaptic endings of the neuron overlap with postsynaptic endings of other neurons.

In some embodiments, provided is a homogenous population of terminally differentiated neurons derived from pluripotent stem cells, wherein at least about any one of: 50%, 55%, 60%, 65%, 70%, 75%, 80%, 85%, 90%, 91%, 92%, 93%, 94%, 95%, 96%, 97%, or 98% of the neurons express two or more pre-synaptic markers selected from vGLUT2, Synapsin 1, and Synapsin 2. In some embodiments, provided is a homogenous population of terminally differentiated neurons derived from pluripotent stem cells, wherein at least about 95% of the neurons express two or more pre-synaptic markers selected from vGLUT2, Synapsin 1, and Synapsin 2. In some embodiments, at least about any one of: 50%, 55%, 60%, 65%, 70%, 75%, 80%, 85%, 90%, 91%, 92%, 93%, 94%, 95%, 96%, 97%, or 98% of the neurons express two or more post-synaptic markers selected from: PSD95, SHANK, PanSHANK, GluR1, GluR2, PanSAPAP, and NR1. In some embodiments, provided is a homogenous population of terminally differentiated neurons derived from pluripotent stem cells, wherein at least about 95% of the neurons express two or more post-synaptic markers selected from: PSD95, SHANK, PanSHANK, GluR1, GluR2, PanSAPAP, and NR1.

In some embodiments, provided is a homogenous population of terminally differentiated neurons derived from pluripotent stem cells, wherein at least about any one of: 50%, 55%, 60%, 65%, 70%, 75%, 80%, 85%, 90%, 91%, 92%, 93%, 94%, 95%, 96%, 97%, or 98% of the neurons express one or more upper-layer cortical neuron markers. In some embodiments, at least about 95% of the neurons express one or more upper-layer cortical neuron markers. In some embodiments, no more than about any one of: 1%, 2%, 3%, 4%, 5%, 6%, 7%, 8%, 9%, 10%, 12%, 15%, 20%, 25%, 30%, 40%, or 50% of the neurons express one or more lower layer cortical neuron markers. In some embodiments, no more than about 5% of the neurons express one or more lower layer cortical neuron markers. In some embodiments, provided is a homogenous population of terminally differentiated neurons derived from pluripotent stem cells, wherein at least about any one of: 50%, 55%, 60%, 65%, 70%, 75%, 80%, 85%, 90%, 91%, 92%, 93%, 94%, 95%, 96%, 97%, or 98% of the neurons express CUX2. In some embodiments, at least about 95% of the neurons express CUX2. In some embodiments, no more than about any one of: 1%, 2%, 3%, 4%, 5%, 6%, 7%, 8%, 9%, 10%, 12%, 15%, 20%, 25%, 30%, 40%, or 50% of the neurons express CTIP2 and/or SATB2. In some embodiments, no more than about 5% of the neurons express CTIP2 and/or SATB2.

In some embodiments, the neurons express representative markers for dendrites, cell bodies, axons and synapses in highly replicable manner. In some embodiments, the expressions of dendritic marker MAP2, cell body marker CUX2, axon marker Tau, and/or synapse marker Synapsin 1/2 in neurons are highly replicable across replicate experiments. In some embodiments, the expressions of dendritic marker MAP2, cell body marker CUX2, axon marker Tau, and/or synapse marker Synapsin 1/2 in neurons are highly replicable across replicate experiments, wherein the z-factor for one or more of MAP2, CUX2, Tau and Synapsin 1/2 is at least about 0.1, 0.2, 0.3, 0.4, 0.5, or 0.6. In some embodiments, the expressions of dendritic marker MAP2, cell body marker CUX2, axon marker Tau, and/or synapse marker Synapsin 1/2 in neurons are highly replicable across replicate experiments, wherein the z-factor for each of MAP2, CUX2, Tau and Synapsin 1/2 is at least 0.4.

In some embodiments, the homogenous population of terminally differentiated neurons is derived in a process comprising: (a) differentiating NSCs into NSC-derived neurons; (b) replating the NSC-derived neurons in presence of primary human astrocytes; (c) differentiating and maturing the PSC-derived neurons for at least about 60 to about 90 days in an automated cell culture system. In some embodiments, the method comprises: (a) culturing the NSCs under conditions to increase the levels of NGN2 and ASCL1, in combination with a cell cycle inhibitor for at least about 7 days, thereby generating NSC-derived neurons; (b) replating the NSC-derived neurons in presence of primary human astrocytes; (c) differentiating and maturing the NSC-derived neurons for at least about 60 to about 90 days in an automated cell culture system.

In some embodiments, provided is a homogenous population of terminally differentiated neurons from pluripotent stem cells (PSCs). In some embodiments, the homogenous population of terminally differentiated neurons is derived in a process comprising: (a) generating a pluripotent stem cell- (PSC-) derived neural stem cell (NSC) line expressing NGN2, and ASCL1 under an inducible system; (b) culturing the NSC line under conditions to induce the expression of NGN2 and ASCL1, in combination with a cell cycle inhibitor for at least about 7 days, thereby generating PSC-derived neurons; (c) replating the PSC-derived neurons in presence of primary human astrocytes; and/or (d) differentiating and/or maturing the PSC-derived neurons for at least about 60 to about 90 days in an automated cell culture system.

In some embodiments, the step of deriving the homogenous population of terminally differentiated neurons comprises differentiating and/or maturing the PSC-derived neurons in any one of the automated cell culture systems described above. In some embodiments, the step of differentiating and/or maturing the NSC-derived neurons comprises differentiating and/or maturing the NSC-derived neurons in any one of the automated cell culture systems described above.

In some embodiments according to any one of the homogenous populations of terminally differentiated neurons described herein, the automated culture media aspiration comprises aspiration with a pipet tip, further wherein: (a) the distal end of the pipet tip is at about 0.8 mm to about 1.2 mm above the bottom surface of the well before, during and/or after the aspiration; (b) the pipet tip is at an angle of about 800 to about 90° to the bottom surface of the well before, during and/or after the aspiration; (c) the pipet tip has a displacement of no more than 0.2 mm from the center of the well before, during and/or after the aspiration; optionally wherein the pipet tip is at the center of the well before, during and/or after the aspiration (no displacement); (e) the speed of media aspiration is no more than about 15 μl/s; (f) the start of media aspiration is about 100 ms to about 500 ms subsequent to the pipet tip being placed 1 mm above the bottom surface of the well; (g) the pipet tip is inserted into the well at a speed of about 1 mm/s to about 10 mm/s prior to aspiration; and/or (h) the pipet tip is withdrawn from the well at a speed of about 1 mm/s to about 10 mm/s after aspiration.

In some embodiments according to any one of the homogenous populations of terminally differentiated neurons described herein, the automated culture media aspiration comprises aspiration with a pipet tip, further wherein: (a) the distal end of the pipet tip is at about 1 mm above the bottom surface of the well before, during and/or after the aspiration; (b) the pipet tip is at an angle of about 90° to the bottom surface of the well before, during and/or after the aspiration; (c) the pipet tip has a displacement of no more than 0.1 mm from the center of the well before, during and/or after the aspiration; optionally wherein the pipet tip is at the center of the well before, during and/or after the aspiration (no displacement); (e) the speed of media aspiration is no more than about 7.5 μl/s; (f) the start of media aspiration is about 200 ms subsequent to the pipet tip being placed 1 mm above the bottom surface of the well; (g) the pipet tip is inserted into the well at a speed of about 5 mm/s prior to aspiration; and/or (h) the pipet tip is withdrawn from the well at a speed of about 5 mm/s after aspiration.

In some embodiments according to any one of the homogenous populations of terminally differentiated neurons described herein, the automated culture media replenishment comprises dispensing media with a pipet tip, further wherein: (a) the distal end of the pipet tip is at about 0.8 mm to about 1.2 mm above the bottom surface of the well before the dispensing; (b) the distal end of the pipet tip is withdrawn from the well at about 1 mm/s during the dispensing; (c) the pipet tip is at an angle of about 800 to about 90° to the bottom surface of the well before and/or during the dispensing; (d) the pipet tip has a displacement of no more than 0.2 mm from the center of the well before, and/or during the dispensing, optionally wherein the pipet tip is at the center of the well before, and/or during the dispensing (no displacement); (e) the pipet tip is displaced (such as displaced laterally) to contact a first side of the well about 0.8 mm to about 1.2 mm from the center in a first direction, at a height of about 10 mm to about 15 mm above the bottom of the well at a speed of about 50 mm/s to about 200 mm/s; (f) the pipet tip is displaced (such as displaced laterally) to contact a second side of the well about 0.8 mm to about 1.2 mm from the center in a second direction, at a height of about 10 mm to about 15 mm above the bottom of the well at a speed of about 50 mm/s to about 200 mm/s, optionally wherein the first direction is at an angle of about 1600 to about 2000 to the second direction; (g) the speed of media dispensing is no more than about 5 μl/s; (h) the acceleration of media dispensing is about 200 μl/s² to about 1000 μl/s²; (i) the deceleration of media dispensing is about 200 μl/s² to about 1000 μl/s²; (j) the start of media dispensing is about 100 ms to about 500 ms subsequent to the pipet tip being placed 1 mm above the bottom surface of the well; (k) the pipet tip is inserted into the well at a speed of about 1 mm/s to about 10 mm/s prior to dispensing; and/or (l) the pipet tip is withdrawn from the well at a speed of about 1 mm/s to about 10 mm/s after dispensing. In some embodiments, the pipet tip is displaced (such as displaced laterally) before, during and/or after the dispensing. In some embodiments, the pipet tip is displaced laterally during the dispensing. In some embodiments, the pipet tip is displaced laterally after the dispensing. In some embodiments, the pipet tip is displaced laterally before and/or during being withdrawn from the well.

In some embodiments according to any one of the homogenous populations of terminally differentiated neurons described herein, the automated culture media replenishment comprises dispensing media with a pipet tip, further wherein: (a) the distal end of the pipet tip is at about 1 mm above the bottom surface of the well before the dispensing; (b) the distal end of the pipet tip is withdrawn from the well at about 1 mm/s during the dispensing; (c) the pipet tip is at an angle of about 90° to the bottom surface of the well before and/or during the dispensing; (d) the pipet tip has a displacement of no more than 0.1 mm from the center of the well before, and/or during the dispensing, optionally wherein the pipet tip is at the center of the well before, and/or during the dispensing (no displacement); (e) the pipet tip is displaced (such as displaced laterally) to contact a first side of the well about 1 mm from the center in a first direction, at a height of about 12.40 mm above the bottom of the well at a speed of about 100 mm/s; (f) the pipet tip is displaced (such as displaced laterally) to contact a second side of the well about 1 mm from the center in a second direction, at a height of about 12.40 mm above the bottom of the well at a speed of about 100 mm/s, optionally wherein the first direction is at an angle of about 1800 to the second direction; (g) the speed of media dispensing is no more than about 1.5 μl/s; (h) the acceleration of media dispensing is about 500 μl/s²; (i) the deceleration of media dispensing is about 500 μl/s²; (j) the start of media dispensing is about 200 ms subsequent to the pipet tip being placed 1 mm above the bottom surface of the well; (k) the pipet tip is inserted into the well at a speed of about 5 mm/s prior to dispensing; and/or (l) the pipet tip is withdrawn from the well at a speed of about 5 mm/s after dispensing. In some embodiments, the pipet tip is displaced (such as displaced laterally) before, during and/or after the dispensing. In some embodiments, the pipet tip is displaced laterally during the dispensing. In some embodiments, the pipet tip is displaced laterally after the dispensing. In some embodiments, the pipet tip is displaced laterally before and/or during being withdrawn from the well.

In some embodiments, wherein the cell culture system comprises one or more batches of 384-well plates, wherein each batch comprises up to twenty-five 384-well plates arranged in 5 columns and 5 rows; the automated cell culture system comprises automated discarding of up to 25 corresponding used racks of 384-pipet tips and automated engagement of up to 25 corresponding new racks of 384-pipet tips subsequent to each round of media aspiration. In some embodiments, wherein the cell culture system comprises one or more batches of 384-well plates, wherein each batch comprises up to twenty-five 384-well plates arranged in 5 columns and 5 rows; the automated cell culture system comprises automated discarding of up to 25 corresponding used racks of 384-pipet tips and automated engagement of up to 25 corresponding new racks of 384-pipet tips subsequent to each round of media dispensing.

In some embodiments according to any one of the homogenous populations of terminally differentiated neurons described herein, the method comprises about any one of 1, 2, 3, 4, 5, 6, 7, 8, 9, 10, 12, 15, 18, 20 or 25 rounds of automated culture media replacements. In some embodiments, the time interval between two rounds of culture media replacements is about any one of: 1, 2, 3, 4, 5, 6, 7, 8, 9, or 10 days. In some embodiments, the time interval between two successive rounds of culture media replacements is about any one of: 1, 2, 3, 4, 5, 6, 7, 8, 9, or 10 days. In some embodiments, the time interval between two rounds of culture media replacements is about 3 or 4 days. In some embodiments, the time interval between two successive rounds of culture media replacements is about 3 or 4 days.

In some embodiments according to any one of the homogenous populations of terminally differentiated neurons described herein, about any one of: 30%, 40%, 50%, 60%, 70%, or 80% of culture media is replaced in one or more rounds of culture media replacement. In some embodiments, about any one of: 40%, 42%, 44%, 46%, 48%, 50%, 52%, 54%, 56%, 58%, or 60% of culture media is replaced in one or more rounds of culture media replacement. In some embodiments, any one of about: 30% to 40%, 40% to 50%, 50% to 60%, 60% to 70%, or 70% to 80% of culture media is replaced in one or more rounds of culture media replacement. In some embodiments, about 50% of culture media is replaced in one or more rounds of culture media replacement.

In some embodiments according to any one of the homogenous populations of terminally differentiated neurons described herein, about any one of: 30%, 40%, 50%, 60%, 70%, or 80% of culture media is replaced in each round of culture media replacement. In some embodiments, about any one of: 40%, 42%, 44%, 46%, 48%, 50%, 52%, 54%, 56%, 58%, or 60% of culture media is replaced in each round of culture media replacement. In some embodiments, any one of about: 30% to 40%, 40% to 50%, 50% to 60%, 60% to 70%, or 70% to 80% of culture media is replaced in each round of culture media replacement. In some embodiments, about 50% of culture media is replaced in each round of culture media replacement.

The use of any one of the homogenous populations of terminally differentiated neurons described herein for use in modeling neurodegenerative diseases, wherein the culture system comprises substantially defined culture media and wherein the culture system is amenable to modular and tunable inputs of: one or more disease-associated components and/or one or more neuroprotective components.

### Neuronal Culture for Modeling Neurodegenerative Disease and Uses Thereof

### Alzheimer's Disease Modeling

Alzheimer's disease (AD) is characterized by the pathological hallmarks of amyloid-O (AD) plaques, neurofibrillary tangles, astrogliosis, and neuronal loss. The accuracy of an AD model can be improved by using terminally differentiated neurons that are more translationally relevant, as well as a system that allows for modular (allowing for efficient addition or removal of components throughout modeling) and tunable (allowing for efficient control of amounts of components) input of disease-causing and neuroprotective factors. A highly modular and tunable system is difficult, if not impossible to achieve in an in vivo AD model. Three-dimensional (3D) AD organoid model systems can allow for certain extents of manipulations, but in some instance may lack the precise control in rapidly tuning the disease-causing and/or neuroprotective factors, as well as present more obstacles in imaging, analysis and screening. In this disclosure, provided is a quantitative, high throughput, multiplexed, systematic, and reproducible in vitro AD model system to allow for pharmacological studies, mechanistic studies, and screening efforts. Prior to this disclosure, such a novel, high throughput human iPSC-based model of AD recapitulates key hallmark pathologies that have been historically difficult to replicate in one model system. The system described herein can be deployed in a 2D tissue culture format that facilities high-throughput automation in tissue culture and image analysis. Provided is a model system with a demonstration of key hallmark pathologies of AD, as well as the first demonstration, in in vitro with 2D human iPSC culture, of certain hallmarks such as robust neuritic plaque-like formation.

### Neuronal Culture System for Modeling Neurodegenerative Disease

In some aspects, provided is a neuronal culture system for use in modeling neurodegenerative diseases, wherein the culture system comprises substantially defined culture media and wherein the culture system is amenable to modular and tunable inputs of: one or more disease-associated components and/or one or more neuroprotective components. In some embodiments, the neuronal culture system is a neural stem cell-derived. In some embodiments, the neuronal culture system is a pluripotent stem cell-derived. In some embodiments, provided is a neuronal culture system for use in modeling neurodegenerative diseases, wherein the culture system comprises substantially defined culture media and wherein the culture system is amenable to modular and tunable inputs of: one or more disease-associated components and/or one or more neuroprotective components.

In some embodiments, the neurodegenerative disease is Alzheimer's disease. In some embodiments according to any one of the neuronal culture systems described herein, wherein the neurodegenerative disease is Alzheimer's disease, the disease-associated components comprises soluble Aβ species. In some embodiments, the disease-associated component comprises overexpression of mutant APP, optionally wherein the disease-associated component comprises inducible overexpression of mutant APP. In some embodiments, the disease-associated component comprises pro-inflammatory cytokine. In some embodiments, the neuroprotective component comprises anti-Aβ antibody. In some embodiments, the neuroprotective component comprises DLK inhibitor, GSK3D inhibitor, CDK5 inhibitor, JNK inhibitor and/or Fvn inhibitor. In some embodiments, the neuroprotective component comprises microglia.

In some embodiments according to any one of the neuronal culture systems described herein, wherein the neurodegenerative disease is Alzheimer's disease, wherein: (a) the disease-associated components comprises soluble Aβ species; (b) the disease-associated component comprises overexpression of mutant APP, optionally wherein the disease-associated component comprises inducible overexpression of mutant APP; (c) the disease-associated component comprises pro-inflammatory cytokine; (d) the neuroprotective component comprises anti-Aβ antibody; (e) the neuroprotective component comprises DLK inhibitor, GSK3β inhibitor, CDK5 inhibitor, and/or Fvn inhibitor; and/or (f) the neuroprotective component comprises microglia,

In some embodiments, the system does not comprise non-defined culture media. In some embodiments, the system does not comprise non-defined matrix. In some embodiments, the system does not comprise matrigel. In some embodiments, the system comprises culture media that is not completely defined. In some embodiments, the system comprises non-defined matrix. In some embodiments, the system comprises matrigel. In some embodiments, the system comprises completely defined culture media. In some embodiments, the system comprises completely defined matrices.

In some embodiments, the soluble Aβ species comprises soluble Aβ oligomers. In some embodiments, the soluble Aβ species comprises soluble Aβ monomers. In some embodiments, the soluble Aβ species comprises soluble Aβ monomers and soluble Aβ oligomers. In some embodiments, the soluble Aβ species comprises soluble Aβ fibrils, soluble Aβ monomers and/or soluble Aβ oligomers.

In some embodiments according to any one of the neuronal culture systems described herein, wherein the neuronal culture system comprises the disease-associated component comprising soluble Aβ species, the Tau protein in the neuronal culture is hyperphosphorylated in one or more of S396/404, S217, S235, S400/T403/S404, and T181 residues. In some embodiments, wherein the neuronal culture system comprises the disease-associated component comprising soluble Aβ species, the phosphorylation of Tau protein in the neuronal culture at one or more of S396/404, S217, S235, S400/T403/S404, and T181 residues is increased by about any one of: 20%, 50%, 80%, 100%, 2-fold, 3-fold, 4-fold, 5-fold, 8-fold, 10-fold, 15-fold, 20-fold, 25-fold, 30-fold, 40-fold, 50-fold, 100-fold, 500-fold, 1000-fold, 10000-fold, or more, compared to a corresponding neuronal culture system not comprising the soluble Aβ species.

In some embodiments according to any one of the neuronal culture systems described herein, wherein the neuronal culture system comprises the disease-associated component comprising soluble Aβ species, the neuronal culture system displays increased neuronal toxicity as compared to a corresponding neuronal culture system not comprising the soluble Aβ species. In some embodiments, wherein the neuronal culture system comprises the disease-associated component comprising soluble Aβ species, the neuronal toxicity in the neuronal culture system is increased by about any one of: 10%, 20%, 30%, 40%, 50%, 60%, 70%, 80%, 90%, 100%, 2-fold, 3-fold, 4-fold, 5-fold, 8-fold, 10-fold, 15-fold, 20-fold, 25-fold, 30-fold, 40-fold, 50-fold, 100-fold, 500-fold, 1000-fold, 10000-fold, or more as compared to a corresponding neuronal culture system not comprising the soluble Aβ species.

In some embodiments according to any one of the neuronal culture systems described herein, wherein the neuronal culture system comprises the disease-associated component comprising soluble Aβ species, the neuronal culture system displays a decrease of MAP2-positive neurons as compared to a corresponding neuronal culture system not comprising the soluble Aβ species. In some embodiments, wherein the neuronal culture system comprises the disease-associated component comprising soluble Aβ species, the amount of MAP2-positive neurons is decreased by about any one of: 1%, 2%, 5%, 8%, 10%, 20%, 30%, 40%, 50%, 60%, 70%, 80%, 90%, 99% as compared to a corresponding neuronal culture system not comprising the soluble Aβ species. In some embodiments, wherein the neuronal culture system comprises the disease-associated component comprising soluble Aβ species, the amount of MAP2-positive neurons is decreased by 100% as compared to a corresponding neuronal culture system not comprising the soluble Aβ species. In some embodiments, wherein the neuronal culture system comprises the disease-associated component comprising soluble Aβ species, the amount of MAP2-positive neurons is decreased by about any one of: 10-fold, 20-fold, 50-fold, 100-fold, 500-fold, 1000-fold, 10000-fold, 100000-fold or more as compared to a corresponding neuronal culture system not comprising the soluble Aβ species.

In some embodiments according to any one of the neuronal culture systems described herein, wherein the neuronal culture system comprises the disease-associated component comprising soluble Aβ species, the neuronal culture system displays a decrease of synapsin-positive neurons as compared to a corresponding neuronal culture system not comprising the soluble Aβ species. In some embodiments, wherein the neuronal culture system comprises the disease-associated component comprising soluble Aβ species, the amount of synapsin-positive neurons is decreased by about any one of: 1%, 2%, 5%, 8%, 0%, 20%, 30%, 40%, 50%, 60%, 70%, 80%, 90%, 99% as compared to a corresponding neuronal culture system not comprising the soluble Aβ species. In some embodiments, wherein the neuronal culture system comprises the disease-associated component comprising soluble Aβ species, the amount of synapsin-positive neurons is decreased by 100% as compared to a corresponding neuronal culture system not comprising the soluble Aβ species. In some embodiments, wherein the neuronal culture system comprises the disease-associated component comprising soluble Aβ species, the amount of MAP2-positive neurons is decreased by about any one of: 10-fold, 20-fold, 50-fold, 100-fold, 500-fold, 1000-fold, 10000-fold, 100000-fold or more as compared to a corresponding neuronal culture system not comprising the soluble Aβ species. In some embodiments, the synapsin is Synapsin 1 and/or Synapsin 2.

In some embodiments, Aβ induced neurotoxicity phenotypes are dose-dependent and progressive. In some embodiments, higher doses resulted in faster pathology development and neuronal loss.

In some embodiments according to any one of the neuronal culture systems described herein, wherein the neuronal culture system comprises the disease-associated component comprising soluble Aβ species, the neuronal culture system displays an increase in Tau phosphorylation in neurons as compared to a neuronal culture system not comprising the soluble Aβ species, wherein the concentration of Aβ is no less than a first concentration. In some embodiments, the neuronal culture system displays a decrease of synapsin-positive neurons as compared to a neuronal culture system not comprising the soluble Aβ species, wherein the concentration of Aβ is no less than a second concentration. In some embodiments, the culture system displays a decrease of CUX2-positive neurons as compared to neuronal culture system not comprising the soluble Aβ species, wherein the concentration of Aβ is no less than a third concentration. In some embodiments, the culture system displays a decrease of MAP2-positive neurons as compared to neuronal culture system not comprising the soluble Aβ species, wherein Aβ is at no less than a forth concentration. In some embodiments, the neuronal culture system displays an increase in Tau phosphorylation in neurons as compared to a neuronal culture system not comprising the soluble Aβ species, wherein the concentration of Aβ is no less than a first concentration; and/or the neuronal culture system displays a decrease of synapsin-positive neurons as compared to a neuronal culture system not comprising the soluble Aβ species, wherein the concentration of Aβ is no less than a second concentration; and/or the culture system displays a decrease of CUX2-positive neurons as compared to neuronal culture system not comprising the soluble Aβ species, wherein the concentration of Aβ is no less than a third concentration; and/or the culture system displays a decrease of MAP2-positive neurons as compared to neuronal culture system not comprising the soluble Aβ species, wherein Aβ is at no less than a forth concentration. In some embodiments, the concentration of Aβ is determined by the concentration of Aβ fibrils. In some embodiments, the concentration of Aβ is determined by the concentration of soluble Aβ species. In some embodiments, the concentration of Aβ is determined by the concentration of soluble Aβ species and/or Aβ fibrils.

In some embodiments according to any one of the neuronal culture systems described above, the first concentration is higher than the second, third and fourth concentrations; and/or the second concentration is higher than the third and fourth concentrations; and/or the third concentration is higher than the fourth concentration. In some embodiments, the first concentration is about 2 μM to about 20 μM. In some embodiments, the first concentration is about any one of: 2, 3, 4, 5, 6, 7, 8, 9 10, 12, 14, 16, 18 or 20 μM. In some embodiments, the second concentration is about 5 μM. In some embodiments, the second concentration is about 1 μM to about 10 μM. In some embodiments, the second concentration is about any one of: 1, 2, 2.5, 3, 4, 5, 6, 7, 8, 9 or 10 μM. In some embodiments, the second concentration is about 2.5 μM. In some embodiments, the third concentration is about 0.25 μM to about 5 μM. In some embodiments, the third concentration is any one of about 0.25, 0.5, 0.75, 1, 1.25, 1.5, 1.75, 2, 2.5, 3, 3.5, 4, 4.5 or 5 μM. In some embodiments, the third concentration is about 1.25 μM. In some embodiments, the fourth concentration is about 0.05 μM to about 2 μM. In some embodiments, the third concentration is any one of about 0.05, 0.1, 0.2, 0.3, 0.4, 0.5, 0.6, 0.7, 0.8, 0.9, 1.0, 1.2, 1.4, 1.6, 1.8 or 2.0 μM. In some embodiments, the third concentration is about 0.3 μM. In some embodiments, the fourth concentration is any one of about 0.05, 0.1, 0.2, 0.3, 0.4, 0.5, 0.6, 0.7, 0.8, 0.9, 1.0, 1.2, 1.4, 1.6, 1.8 or 2.0 μM. In some embodiments, the fourth concentration is about 0.3 μM. In some embodiments, the neurons are contacted with the described concentration of Aβ for about any one of 1, 2, 3, 4, 5, 6, 7, 8, 9, 10, 11, 12, 13, 14, 15, 16, 17, 18, 19, 20, 21, 24, 28, 30, 35, 40, 50, or 60 days. In some embodiments, the neurons are contacted with the described concentration of Aβ for about 7, 14 or 21 days.

In some embodiments, wherein the neuronal culture system comprises the disease-associated component comprising soluble Aβ species, the neuronal culture system displays an increase in Tau phosphorylation in neurons as compared to a neuronal culture system not comprising the soluble Aβ species, wherein the concentration of Aβ is no less than a first concentration; and/or the neuronal culture system displays a decrease of synapsin-positive neurons as compared to a neuronal culture system not comprising the soluble Aβ species, wherein the concentration of Aβ is no less than a second concentration; and/or the culture system displays a decrease of CUX2-positive neurons as compared to neuronal culture system not comprising the soluble Aβ species, wherein the concentration of Aβ is no less than a third concentration; and/or the culture system displays a decrease of MAP2-positive neurons as compared to neuronal culture system not comprising the soluble Aβ species, wherein Aβ is at no less than a forth concentration, further wherein the first concentration is higher than the second, third and fourth concentrations; and/or the second concentration is higher than the third and fourth concentrations; and/or the third concentration is higher than the fourth concentration.

In some embodiments, according to any one of the neuronal culture systems described herein, wherein the neuronal culture system comprises the disease-associated component comprising soluble Aβ species, the neurons are contacted with the disease-associated component Aβ for about any one of: 1, 2, 3, 4, 5, 6, 7, 8, 9, 10, 11, 12, 13, 14, 15, 16, 17, 18, 19, 20, 21, 24, 28, 30, 35, 40, 50, or 60 days. In some embodiments, the neurons are contacted with about any one of: 0.05, 0.1, 0.2, 0.3, 0.4, 0.5, 0.6, 0.7, 0.8, 0.9, 1, 1.2, 1.4, 1.6, 1.8 or 2, 3, 4, 5, 6, 7, 8, 9 10, 12, 14, 16, 18 or 20 μM Aβ for about any one of: 1, 2, 3, 4, 5, 6, 7, 8, 9, 10, 11, 12, 13, 14, 15, 16, 17, 18, 19, 20, 21, 24, 28, 30, 35, 40, 50, or 60 days.

In some embodiments according to any one of the neuronal culture systems described herein, wherein the neuronal culture system comprises the disease-associated component comprising soluble Aβ species, wherein the neuronal culture system further comprises astrocytes in co-culture, the astrocytes exhibit increased GFAP expression as compared to astrocytes co-cultured in a corresponding neuronal culture system not comprising the soluble Aβ species. In some embodiments according to any one of the neuronal culture systems described herein, wherein the neuronal culture system comprises the disease-associated component comprising soluble Aβ species, wherein the neuronal culture system further comprises astrocytes in co-culture, the astrocytes exhibit increased GFAP fragmentation as compared to astrocytes co-cultured in a corresponding neuronal culture system not comprising the soluble Aβ species.

In some embodiments, wherein the neuronal culture system comprises the disease-associated component comprising soluble Aβ species, wherein the neuronal culture system further comprises astrocytes in co-culture, the astrocytes exhibit an increased in GFAP expression by about any one of: 10%, 20%, 30%, 40%, 50%, 60%, 70%, 80%, 90%, 100%, 2-fold, 3-fold, 4-fold, 5-fold, 8-fold, 10-fold, 15-fold, 20-fold, 25-fold, 30-fold, 40-fold, 50-fold, 100-fold, 500-fold, 1000-fold, 10000-fold, or more as compared to astrocytes co-cultured in a corresponding neuronal culture system not comprising the soluble Aβ species. In some embodiments, wherein the neuronal culture system comprises the disease-associated component comprising soluble Aβ species, wherein the neuronal culture system further comprises astrocytes in co-culture, the astrocytes exhibit an increased in GFAP fragmentation by about any one of: 10%, 20%, 30%, 40%, 50%, 60%, 70%, 80%, 90%, 100%, 2-fold, 3-fold, 4-fold, 5-fold, 8-fold, 10-fold, 15-fold, 20-fold, 25-fold, 30-fold, 40-fold, 50-fold, 100-fold, 500-fold, 1000-fold, 10000-fold, or more as compared to astrocytes co-cultured in a corresponding neuronal culture system not comprising the soluble Aβ species.

In some embodiments according to any one of the neuronal culture systems described herein, wherein the neuronal culture system comprises the disease-associated component comprising soluble Aβ species, the neuronal culture system exhibits Methoxy X04-positive Aβ plaques or plaque-like structures. In some embodiments, wherein the neuronal culture system comprises the disease-associated component comprising soluble Aβ species, the neuronal culture system exhibits an increase in Methoxy X04-positive Aβ plaques or plaque-like structures as compared to a corresponding neuronal culture system not comprising the soluble Aβ species. In some embodiments, wherein the neuronal culture system comprises the disease-associated component comprising soluble Aβ species, the neuronal toxicity in the neuronal culture system is increased the neuronal culture system exhibits an increase in Methoxy X04-positive Aβ plaques or plaque-like structures by about any one of: 10%, 20%, 30%, 40%, 50%, 60%, 70%, 80%, 90%, 100%, 2-fold, 3-fold, 4-fold, 5-fold, 8-fold, 10-fold, 15-fold, 20-fold, 25-fold, 30-fold, 40-fold, 50-fold, 100-fold, 500-fold, 1000-fold, 10000-fold, or more as compared to a corresponding neuronal culture system not comprising the soluble Aβ species. In some embodiments, at least a subset of the Methoxy X04-positive A D plaques or plaque-like structures are surrounded by neurites. In some embodiments, at least about any one of: 10%, 20%, 30%, 40%, 50%, 60%, 70%, 80%, 90%, 95% or 99% of the Methoxy X04-positive Aβ plaques or plaque-like structures are surrounded by neurites. In some embodiments, at least a subset of the Methoxy X04-positive Aβ plaques or plaque-like structures are surrounded by neurites, wherein the neurites are marked by neurofilament heavy chain (NFL-H) axonal swelling and/or phosphoriylated Tau (S235) positive blebbings. In some embodiments, at least a subset of the Methoxy X04-positive Aβ plaques or plaque-like structures are surrounded by neurites, wherein the neurites are marked by neurofilament heavy chain (NFL-H) axonal swelling and/or phosphorylated Tau (S235) positive blebbings, wherein the neurites are dystrophic. In some embodiments according to any one of the neuronal culture systems described herein, the plaques or plaque-like structures surrounded by neurites exhibit ApoE expression localized in the amyloid plaques. In some embodiments, the plaques or plaque-like structures surrounded by neurites exhibit APP in the membranes of the dystrophic neurites. In some embodiments, the plaques or plaque-like structures surrounded by neurites exhibit ApoE expression localized in the amyloid plaques and APP in the membranes of the dystrophic neurites. In some embodiments, the neurites are dystrophic.

In some embodiments, wherein the neuronal culture system comprises the disease-associated component comprising soluble Aβ species, the neuronal culture system exhibits neuritic dystrophy. In some embodiments, wherein the neuronal culture system comprises the disease-associated component comprising soluble Aβ species, the neuronal culture system exhibits neuritic dystrophy, the neuronal culture system exhibits an increase in neuritic dystrophy as compared to a corresponding neuronal culture system not comprising the soluble Aβ species. In some embodiments, wherein the neuronal culture system comprises the disease-associated component comprising soluble Aβ species, the neuronal culture system exhibits neuritic dystrophy, the neuronal culture system exhibits an increase in neuritic dystrophy by about any one of: 10%, 20%, 30%, 40%, 50%, 60%, 70%, 80%, 90%, 100%, 2-fold, 3-fold, 4-fold, 5-fold, 8-fold, 10-fold, 15-fold, 20-fold, 25-fold, 30-fold, 40-fold, 50-fold, 100-fold, 500-fold, 1000-fold, 10000-fold, or more as compared to a corresponding neuronal culture system not comprising the soluble Aβ species.

In some embodiments according to any one of the neuronal culture systems described herein, the culture system comprises the disease-associated component comprising soluble Aβ species; the disease-associated component comprising neuroinflammatory cytokine, and the neuroprotective component comprising microglia. In some embodiments, the culture system comprises the disease-associated component comprising soluble Aβ species; the disease-associated component neuroinflammatory cytokine, and the neuroprotective component microglia. In some embodiments, the microglia is derived from pluripotent stem cells (such as but not limited to embryonic stem cells or induced pluripotent stem cells). In some embodiments, the microglia expresses one or more of TREM2, TMEM 119, CXCR1, P2RY12, PU.1, MERTK, CD33, CD64, CD32 and IBA-1. In some embodiments, the microglia is iPSC-derived microglia and expresses one or more of TREM2, TMEM 119, CXCR1, P2RY12, PU.1 MERTK, CD33, CD64, CD32 and IBA-1.

In some embodiments, wherein the neuronal culture system comprises (1) soluble Aβ species, and (2) microglia, the neuronal culture system exhibits decreased neuronal toxicity as compared to a corresponding neuronal culture system not comprising microglia. In some embodiments, wherein the neuronal culture system culture system comprises (1) soluble Aβ species, and (2) microglia, the neuronal culture system exhibits about any one of: 1%, 2%, 5%, 8%, 10%, 15%, 20%, 25%, 30%, 40%, 50%, 60%, 70%, 80%, 90%, or 99% decrease in neuronal toxicity as compared to a corresponding neuronal culture system not comprising microglia. In some embodiments, wherein the neuronal culture system culture system comprises (1) soluble Aβ species, and (2) microglia, the neuronal culture system exhibits about 25% decrease in neuronal toxicity as compared to a corresponding neuronal culture system not comprising microglia.

In some embodiments, wherein the neuronal culture system comprises (1) soluble Aβ species, and (2) microglia, the neuronal culture system exhibits increased microglial-Aβ plaque association and/or increased Aβ plaque formation as compared to a corresponding neuronal culture system not comprising microglia. In some embodiments, wherein the neuronal culture system comprises (1) soluble Aβ species, and (2) microglia, the neuronal culture system exhibits an increase in microglial-Aβ plaque association by about any one of: 10%, 20%, 30%, 40%, 50%, 60%, 70%, 80%, 90%, 100%, 2-fold, 3-fold, 4-fold, 5-fold, 8-fold, 10-fold, 15-fold, 20-fold, 25-fold, 30-fold, 40-fold, 50-fold, 100-fold, 500-fold, 1000-fold, 10000-fold, or more as compared to a corresponding neuronal culture system not comprising microglia. In some embodiments, wherein the neuronal culture system comprises (1) soluble Aβ species, and (2) microglia, the neuronal culture system exhibits an increase in Aβ plaque formation by about any one of 10%, 20%, 30%, 40%, 50%, 60%, 70%, 80%, 90%, 100%, 2-fold, 3-fold, 4-fold, 5-fold, 8-fold, 10-fold, 15-fold, 20-fold, 25-fold, 30-fold, 40-fold, 50-fold, 100-fold, 500-fold, 1000-fold, 10000-fold, or more as compared to a corresponding neuronal culture system not comprising microglia,

In some embodiments according to any one of the neuronal culture systems described herein, the culture system comprises the disease-associated component comprising soluble Aβ species; and the neuroprotective component comprising microglia. In some embodiments, the culture system comprises the disease-associated component comprising soluble Aβ species; and the neuroprotective component microglia. In some embodiments, the microglia is iPSC-derived microglia and expresses one or more of: TREM2, TMEM 119, CXCR1, P2RY12, PU.1, MERTK, CD33, CD64, CD32 and IBA-1.

In some embodiments, wherein the neuronal culture system comprises (1) soluble Aβ species, (2) neuroinflammatory cytokine and (3) microglia, the neuronal culture system exhibits increased microglial-Aβ plaque association and/or increased Aβ plaque formation as compared to a corresponding neuronal culture system not comprising microglia. In some embodiments, wherein the neuronal culture system comprises (1) soluble Aβ species, (2) neuroinflammatory cytokine and (3) microglia, the neuronal culture system exhibits an increase in microglial-Aβ plaque association by about any one of: 10%, 20%, 30%, 40%, 50%, 60%, 70%, 80%, 90%, 100%, 2-fold, 3-fold, 4-fold, 5-fold, 8-fold, 10-fold, 15-fold, 20-fold, 25-fold, 30-fold, 40-fold, 50-fold, 100-fold, 500-fold, 1000-fold, 10000-fold, or more as compared to a corresponding neuronal culture system not comprising microglia. In some embodiments, wherein the neuronal culture system comprises (1) soluble Aβ species, and (2) microglia, the neuronal culture system exhibits an increase in Aβ plaque formation by about any one of: 10%, 20%, 30%, 40%, 50%, 60%, 70%, 80%, 90%, 100%, 2-fold, 3-fold, 4-fold, 5-fold, 8-fold, 15-fold, 20-fold, 25-fold, 30-fold, 40-fold, 50-fold, 100-fold, 500-fold, 1000-fold, 10000-fold, or more as compared to a corresponding neuronal culture system not comprising microglia.

In some embodiments, wherein the neuronal culture system comprises (1) soluble Aβ species, (2) neuroinflammatory cytokine and (3) microglia, the neuronal culture system exhibits increased microglial-Aβ plaque association and/or increased Aβ plaque formation as compared to a corresponding neuronal culture system not comprising microglia. In some embodiments, wherein the neuronal culture system comprises (1) soluble Aβ species, (2) neuroinflammatory cytokine and (3) microglia, the neuronal culture system exhibits less than about any one of: 1%, 2%, 5%, 8%, 10%, 15%, 20%, or 30% change in neuronal toxicity as compared to a corresponding neuronal culture system not comprising microglia.

In some embodiments, wherein the neuronal culture system comprises (1) soluble Aβ species, (2) neuroinflammatory cytokine and (3) microglia, the neuronal culture system exhibits increased microglial-Aβ plaque association and/or increased Aβ plaque formation as compared to a corresponding neuronal culture system not comprising microglia. In some embodiments, wherein the neuronal culture system comprises (1) soluble Aβ species, (2) neuroinflammatory cytokine and (3) microglia, the neuronal culture system exhibits less than about 10% change in neuronal toxicity as compared to a corresponding neuronal culture system not comprising microglia.

In some embodiments, wherein the neuronal culture system comprises (1) soluble Aβ species, and (2) anti-Aβ antibody, the neuronal culture system exhibits decreased neuronal toxicity as compared to a corresponding neuronal culture system not comprising anti-Aβ antibody. In some embodiments, wherein the neuronal culture system culture system comprises (1) soluble Aβ species, and (2) anti-Aβ antibody, the neuronal culture system exhibits about any one of: 1%, 2%, 5%, 8%, 10%, 15%, 20%, 25%, 30%, 40%, 50%, 60%, 70%, 80%, 90%, or 99% decrease in neuronal toxicity as compared to a corresponding neuronal culture system not comprising anti-Aβ antibody. In some embodiments, wherein the neuronal culture system culture system comprises (1) soluble Aβ species, and (2) anti-Aβ antibody, the neuronal culture system exhibits about 50% to about 99% decrease in neuronal toxicity as compared to a corresponding neuronal culture system not comprising anti-Aβ antibody.

In some embodiments, wherein the neuronal culture system comprises (1) soluble Aβ species, and (2) anti-Aβ antibody, the neuronal culture system exhibits decreased p-Tau induction as compared to a corresponding neuronal culture system not comprising anti-Aβ antibody. In some embodiments, wherein the neuronal culture system culture system comprises (1) soluble Aβ species, and (2) anti-Aβ antibody, the neuronal culture system exhibits about any one of: 1%, 2%, 5%, 8%, 10%, 15%, 20%, 25%, 30%, 40%, 50%, 60%, 70%, 80%, 90%, or 99% decrease in p-Tau induction as compared to a corresponding neuronal culture system not comprising anti-Aβ antibody. In some embodiments, wherein the neuronal culture system culture system comprises (1) soluble Aβ species, and (2) anti-Aβ antibody, the neuronal culture system exhibits about 50% to about 95% decrease in p-Tau induction as compared to a corresponding neuronal culture system not comprising anti-Aβ antibody.

In some embodiments, wherein the neuronal culture system comprises (1) soluble Aβ species, and (2) anti-Aβ antibody, the neuronal culture system exhibits increased level of MAP2 and/or synapsin as compared to a corresponding neuronal culture system not comprising anti-Aβ antibody. In some embodiments, wherein the neuronal culture system culture system comprises (1) soluble Aβ species, and (2) anti-Aβ antibody, the neuronal culture system exhibits about any one of: 10%, 20%, 30%, 40%, 50%, 60%, 70%, 80%, 90%, 100%, 2-fold, 3-fold, 5-fold, 10-fold, 20-fold, 50-fold, 100-fold, 500-fold, 1000-fold, 10000-fold, 100000-fold increase in level of MAP2 and/or synapsin as compared to a corresponding neuronal culture system not comprising anti-Aβ antibody. In some embodiments, wherein the neuronal culture system culture system comprises (1) soluble Aβ species, and (2) anti-Aβ antibody, the neuronal culture system exhibits about 100-fold increased level of MAP2 and/or synapsin as compared to a corresponding neuronal culture system not comprising anti-Aβ antibody.

In some embodiments according to the neuronal culture system described above, the stoichiometric ratio between anti-Aβ antibodies and soluble Aβ species is about 1:2. In some embodiments according to the neuronal culture system described above, the molar ratio between anti-Aβ antibodies and soluble Aβ species is about 1:2. In some embodiments, the IC50 of synapse rescue is about 1.4 μM anti-Aβ antibodies at about 5 μM soluble Aβ species. In some embodiments, the IC50 of synapse rescue is about 1 μM anti-Aβ antibodies at about 4 μM soluble Aβ species.

In some embodiments, wherein the neuronal culture system comprises (1) soluble Aβ species, and (2) DLK inhibitor, GSK3β inhibitor, CDK5 inhibitor, and/or Fyn kinase inhibitor, the neuronal culture system exhibits decreased neuronal toxicity as compared to a corresponding neuronal culture system not comprising DLK inhibitor, GSK3β inhibitor, CDK5 inhibitor, and/or Fyn kinase inhibitor. In some embodiments, wherein the neuronal culture system culture system comprises (1) soluble Aβ species, and (2) DLK inhibitor, GSK3β inhibitor, CDK5 inhibitor, and/or Fyn kinase inhibitor, the neuronal culture system exhibits about any one of: 1%, 2%, 5%, 8%, 10%, 15%, 20%, 25%, 30%, 40%, 50%, 60%, 70%, 80%, 90%, or 99% decrease in neuronal toxicity as compared to a corresponding neuronal culture system not comprising DLK inhibitor, GSK3β inhibitor, CDK5 inhibitor, or Fyn kinase inhibitor. In some embodiments, wherein the neuronal culture system culture system comprises (1) soluble Aβ species, and (2) DLK inhibitor, GSK3β inhibitor, CDK5 inhibitor, and/or Fyn kinase inhibitor, the neuronal culture system exhibits about 25% decrease in neuronal toxicity as compared to a corresponding neuronal culture system not comprising DLK inhibitor, GSK3β inhibitor, CDK5 inhibitor, or Fyn kinase inhibitor.

In some embodiments according to any one of the neuronal culture systems described herein, the neurons exhibit one or more of: DLK, GSK3, CDK5, JNK and Fyn kinase signaling. In some embodiments, the neuron in said neuronal culture system exhibits DLK signaling at a level that is no more than about 10%, 20%, 30%, 40%, 50%, 60%, 70%, 80%, 90%, 100%, 2-fold, 3-fold, 5-fold, 10-fold, 20-fold lower than a neuron in an Alzheimer's disease patient. In some embodiments, the neuron in said neuronal culture system exhibits GSK3 signaling at a level that is no more than about 10%, 20%, 30%, 40%, 50%, 60%, 70%, 80%, 90%, 100%, 2-fold, 3-fold, 5-fold, 10-fold, 20-fold lower than a neuron in an Alzheimer's disease patient. In some embodiments, the neuron in said neuronal culture system exhibits CDK5 signaling at a level that is no more than about 10%, 20%, 30%, 40%, 50%, 60%, 70%, 80%, 90%, 100%, 2-fold, 3-fold, 5-fold, 10-fold, 20-fold lower than a neuron in an Alzheimer's disease patient. In some embodiments, the neuron in said neuronal culture system exhibits Fyn kinase signaling at a level that is no more than about 10%, 20%, 30%, 40%, 50%, 60%, 70%, 80%, 90%, 100%, 2-fold, 3-fold, 5-fold, 10-fold, 20-fold lower than a neuron in an Alzheimer's disease patient. In some embodiments, the neuron in said neuronal culture system exhibits DLK signaling at a level that is no more than about 10%, 20%, 30%, 40%, 50%, 60%, 70%, 80%, 90%, 100%, 2-fold, 3-fold, 5-fold, 10-fold, 20-fold higher than a neuron in an Alzheimer's disease patient. In some embodiments, the neuron in said neuronal culture system exhibits GSK3 signaling at a level that is no more than about 10%, 20%, 30%, 40%, 50%, 60%, 70%, 80%, 90%, 100%, 2-fold, 3-fold, 5-fold, 10-fold, 20-fold higher than a neuron in an Alzheimer's disease patient. In some embodiments, the neuron in said neuronal culture system exhibits CDK5 signaling at a level that is no more than about 10%, 20%, 30%, 40%, 50%, 60%, 70%, 80%, 90%, 100%, 2-fold, 3-fold, 5-fold, 10-fold, 20-fold higher than a neuron in an Alzheimer's disease patient. In some embodiments, the neuron in said neuronal culture system exhibits Fyn kinase signaling at a level that is no more than about 10%, 20%, 30%, 40%, 50%, 60%, 70%, 80%, 90%, 100%, 2-fold, 3-fold, 5-fold, 10-fold, 20-fold higher than a neuron in an Alzheimer's disease patient. In some embodiments, the neuron in said neuronal culture system exhibits DLK signaling at a level that is at least about 10%, 20%, 30%, 40%, 50%, 60%, 70%, 80%, 90%, 100%, 2-fold, 3-fold, 5-fold, 10-fold, 20-fold higher than a neuron in an Alzheimer's disease patient. In some embodiments, the neuron in said neuronal culture system exhibits GSK3 signaling at a level that is at least about 10%, 20%, 30%, 40%, 50%, 60%, 70%, 80%, 90%, 100%, 2-fold, 3-fold, 5-fold, 10-fold, 20-fold higher than a neuron in an Alzheimer's disease patient. In some embodiments, the neuron in said neuronal culture system exhibits CDK5 signaling at a level that is at least about 10%, 20%, 30%, 40%, 50%, 60%, 70%, 80%, 90%, 100%, 2-fold, 3-fold, 5-fold, 10-fold, 20-fold higher than a neuron in an Alzheimer's disease patient. In some embodiments, the neuron in said neuronal culture system exhibits Fyn kinase signaling at a level that is at least about 10%, 20%, 30%, 40%, 50%, 60%, 70%, 80%, 90%, 100%, 2-fold, 3-fold, 5-fold, 10-fold, 20-fold higher than a neuron in an Alzheimer's disease patient.

In some embodiments according to any one of the neuronal culture systems described herein, the neuronal culture system comprises differentiated neurons, optionally wherein the neuronal culture system comprises homogenous populations of terminally differentiated neurons.

In some embodiments, the neuronal culture system comprises differentiated neurons derived in a process comprising: (a) differentiating NSCs into NSC-derived neurons; (b) replating the NSC-derived neurons in presence of primary human astrocytes; (c) differentiating and maturing the PSC-derived neurons for at least about 60 to about 90 days in an automated cell culture system. In some embodiments, the method comprises: (a) culturing the NSCs under conditions to increase the levels of NGN2 and ASCL1, in combination with a cell cycle inhibitor for at least about 7 days, thereby generating NSC-derived neurons; (b) replating the NSC-derived neurons in presence of primary human astrocytes; (c) differentiating and maturing the NSC-derived neurons for at least about 60 to about 90 days in an automated cell culture system.

In some embodiments, the neuronal culture system comprises differentiated neurons derived in a process comprising: (a) generating a pluripotent stem cell- (PSC-) derived neural stem cell (NSC) line expressing NGN2, and ASCL1 under an inducible system; (b) culturing the NSC line under conditions to induce the expression of NGN2 and ASCL1, in combination with a cell cycle inhibitor for at least about 7 days, thereby generating PSC-derived neurons; (c) replating the PSC-derived neurons in presence of primary human astrocytes; and/or (d) differentiating and/or maturing the PSC-derived neurons for at least about 60 to about 90 days in an automated cell culture system.

In some embodiments, the step of deriving the differentiated neurons comprises differentiating and/or maturing the PSC-derived neurons in any one of the automated cell culture systems described herein. In some embodiments, the step of differentiating and/or maturing the NSC-derived neurons comprises differentiating and/or maturing the NSC-derived neurons in any one of the automated cell culture systems described above.

In some embodiments according to any one of the neuronal culture systems described herein, the automated culture media aspiration comprises aspiration with a pipet tip, further wherein: (a) the distal end of the pipet tip is at about 0.8 mm to about 1.2 mm above the bottom surface of the well before, during and/or after the aspiration; (b) the pipet tip is at an angle of about 800 to about 90° to the bottom surface of the well before, during and/or after the aspiration; (c) the pipet tip has a displacement of no more than 0.2 mm from the center of the well before, during and/or after the aspiration; optionally wherein the pipet tip is at the center of the well before, during and/or after the aspiration (no displacement); (e) the speed of media aspiration is no more than about 15 μl/s; (f) the start of media aspiration is about 100 ms to about 500 ms subsequent to the pipet tip being placed 1 mm above the bottom surface of the well; (g) the pipet tip is inserted into the well at a speed of about 1 mm/s to about 10 mm/s prior to aspiration; and/or (h) the pipet tip is withdrawn from the well at a speed of about 1 mm/s to about 10 mm/s after aspiration.

In some embodiments according to any one of the neuronal culture systems described herein, the automated culture media aspiration comprises aspiration with a pipet tip, further wherein: (a) the distal end of the pipet tip is at about 1 mm above the bottom surface of the well before, during and/or after the aspiration; (b) the pipet tip is at an angle of about 90° to the bottom surface of the well before, during and/or after the aspiration; (c) the pipet tip has a displacement of no more than 0.1 mm from the center of the well before, during and/or after the aspiration; optionally wherein the pipet tip is at the center of the well before, during and/or after the aspiration (no displacement); (e) the speed of media aspiration is no more than about 7.5 μl/s; (f) the start of media aspiration is about 200 ms subsequent to the pipet tip being placed 1 mm above the bottom surface of the well; (g) the pipet tip is inserted into the well at a speed of about 5 mm/s prior to aspiration; and/or (h) the pipet tip is withdrawn from the well at a speed of about 5 mm/s after aspiration.

In some embodiments according to any one of the neuronal culture systems described herein, the automated culture media replenishment comprises dispensing media with a pipet tip, further wherein: (a) the distal end of the pipet tip is at about 0.8 mm to about 1.2 mm above the bottom surface of the well before the dispensing; (b) the distal end of the pipet tip is withdrawn from the well at about 1 mm/s during the dispensing; (c) the pipet tip is at an angle of about 80° to about 90° to the bottom surface of the well before and/or during the dispensing; (d) the pipet tip has a displacement of no more than 0.2 mm from the center of the well before, and/or during the dispensing, optionally wherein the pipet tip is at the center of the well before, and/or during the dispensing (no displacement); (e) the pipet tip is displaced (such as displaced laterally) to contact a first side of the well about 0.8 mm to about 1.2 mm from the center in a first direction, at a height of about 10 mm to about 15 mm above the bottom of the well at a speed of about 50 mm/s to about 200 mm/s; (f) the pipet tip is displaced (such as displaced laterally) to contact a second side of the well about 0.8 mm to about 1.2 mm from the center in a second direction, at a height of about 10 mm to about 15 mm above the bottom of the well at a speed of about 50 mm/s to about 200 mm/s, optionally wherein the first direction is at an angle of about 1600 to about 2000 to the second direction; (g) the speed of media dispensing is no more than about 5 μl/s; (h) the acceleration of media dispensing is about 200 μl/s² to about 1000 μl/s²; (i) the deceleration of media dispensing is about 200 μl/s² to about 1000 μl/s²; (j) the start of media dispensing is about 100 ms to about 500 ms subsequent to the pipet tip being placed 1 mm above the bottom surface of the well; (k) the pipet tip is inserted into the well at a speed of about 1 mm/s to about 10 mm/s prior to dispensing; and/or (l) the pipet tip is withdrawn from the well at a speed of about 1 mm/s to about 10 mm/s after dispensing. In some embodiments, the pipet tip is displaced (such as displaced laterally) before, during and/or after the dispensing. In some embodiments, the pipet tip is displaced laterally during the dispensing. In some embodiments, the pipet tip is displaced laterally after the dispensing. In some embodiments, the pipet tip is displaced laterally before and/or during being withdrawn from the well.

In some embodiments according to any one of the neuronal culture systems described herein, the automated culture media replenishment comprises dispensing media with a pipet tip, further wherein: (a) the distal end of the pipet tip is at about 1 mm above the bottom surface of the well before the dispensing; (b) the distal end of the pipet tip is withdrawn from the well at about 1 mm/s during the dispensing; (c) the pipet tip is at an angle of about 90° to the bottom surface of the well before and/or during the dispensing; (d) the pipet tip has a displacement of no more than 0.1 mm from the center of the well before, and/or during the dispensing, optionally wherein the pipet tip is at the center of the well before, and/or during the dispensing (no displacement); (e) the pipet tip is displaced (such as displaced laterally) to contact a first side of the well about 1 mm from the center in a first direction, at a height of about 12.40 mm above the bottom of the well at a speed of about 100 mm/s; (f) the pipet tip is displaced (such as displaced laterally) to contact a second side of the well about 1 mm from the center in a second direction, at a height of about 12.40 mm above the bottom of the well at a speed of about 100 mm/s, optionally wherein the first direction is at an angle of about 1800 to the second direction; (g) the speed of media dispensing is no more than about 1.5 μl/s; (h) the acceleration of media dispensing is about 500 μl/s²; (i) the deceleration of media dispensing is about 500 μl/s²; (j) the start of media dispensing is about 200 ms subsequent to the pipet tip being placed 1 mm above the bottom surface of the well; (k) the pipet tip is inserted into the well at a speed of about 5 mm/s prior to dispensing; and/or (l) the pipet tip is withdrawn from the well at a speed of about 5 mm/s after dispensing. In some embodiments, the pipet tip is displaced (such as displaced laterally) before, during and/or after the dispensing. In some embodiments, the pipet tip is displaced laterally during the dispensing. In some embodiments, the pipet tip is displaced laterally after the dispensing. In some embodiments, the pipet tip is displaced laterally before and/or during being withdrawn from the well.

In some embodiments, wherein the cell culture system comprises one or more batches of 384-well plates, wherein each batch comprises up to twenty-five 384-well plates arranged in 5 columns and 5 rows; the automated cell culture system comprises automated discarding of up to 25 corresponding used racks of 384-pipet tips and automated engagement of up to 25 corresponding new racks of 384-pipet tips subsequent to each round of media aspiration. In some embodiments, wherein the cell culture system comprises one or more batches of 384-well plates, wherein each batch comprises up to twenty-five 384-well plates arranged in 5 columns and 5 rows; the automated cell culture system comprises automated discarding of up to 25 corresponding used racks of 384-pipet tips and automated engagement of up to 25 corresponding new racks of 384-pipet tips subsequent to each round of media dispensing.

In some embodiments according to any one of the neuronal culture systems described herein, the method comprises about any one of 1, 2, 3, 4, 5, 6, 7, 8, 9, 10, 12, 15, 18, 20 or 25 rounds of automated culture media replacements. In some embodiments, the time interval between two rounds of culture media replacements is about any one of: 1, 2, 3, 4, 5, 6, 7, 8, 9, or 10 days. In some embodiments, the time interval between two successive rounds of culture media replacements is about any one of: 1, 2, 3, 4, 5, 6, 7, 8, 9, or 10 days. In some embodiments, the time interval between two rounds of culture media replacements is about 3 or 4 days. In some embodiments, the time interval between two successive rounds of culture media replacements is about 3 or 4 days.

In some embodiments according to any one of the neuronal culture systems described herein, about any one of: 30%, 40%, 50%, 60%, 70%, or 80% of culture media is replaced in one or more rounds of culture media replacement. In some embodiments, about any one of: 40%, 42%, 44%, 46%, 48%, 50%, 52%, 54%, 56%, 58%, or 60% of culture media is replaced in one or more rounds of culture media replacement. In some embodiments, any one of about: 30% to 40%, 40% to 50%, 50% to 60%, 60% to 70%, or 70% to 80% of culture media is replaced in one or more rounds of culture media replacement. In some embodiments, about 50% of culture media is replaced in one or more rounds of culture media replacement.

In some embodiments according to any one of the neuronal culture systems described herein, about any one of: 30%, 40%, 50%, 60%, 70%, or 80% of culture media is replaced in each round of culture media replacement. In some embodiments, about any one of: 40%, 42%, 44%, 46%, 48%, 50%, 52%, 54%, 56%, 58%, or 60% of culture media is replaced in each round of culture media replacement. In some embodiments, any one of about: 30% to 40%, 40% to 50%, 50% to 60%, 60% to 70%, or 70% to 80% of culture media is replaced in each round of culture media replacement. In some embodiments, about 50% of culture media is replaced in each round of culture media replacement.

### Stem Cells

In some embodiments according to any one of the neuronal cell cultures, methods, and populations of neurons described herein, the neuronal cells (such as neurons) are derived from pluripotent stem cells. As used herein, pluripotent stem cells are cells that have the capacity to self-renew by dividing and to develop into the three primary germ cell layers of the early embryo and therefore into all cells of the adult body. In some embodiments, pluripotent stem cells cannot develop into extra-embryonic tissues such as the placenta. As used herein, pluripotent stem cells can also encompass cells that have potential to develop into the three germ layers as well as extra-embryonic tissues, such as epiblast-derived stem cells. In some embodiments, the pluripotent stem cells are embryonic stem cells. In some embodiments, embryonic stem cells are isolated from embryos (such as human or mouse embryos) and maintained as cell lines. In some embodiments, the pluripotent stem cells are induced pluripotent stem cells (iPSCs). As used herein, an induced pluripotent stem cell can refer to any pluripotent cell obtained by re-programing a non-pluripotent cell. The reprogrammed cell may have been generated by reprogramming a progenitor cell, a partially-differentiated cell, or a fully differentiated cell of any embryonic or extraembryonic tissue lineage. For example, induced pluripotent stem cells can be generated by overexpression of transcription factors (such as including Oct3/4, Sox2, Klf4, c-Myc), in differentiated cells such as fibroblasts. In some embodiments, neurons can be derived from pluripotent stem cells by using combined small molecule inhibition, or activation of transcription factors. In some embodiments, neurons can be derived from pluripotent stem cells by activation of ASCL1 and/or NGN2.

In some embodiments according to any one of the neuronal cell cultures, methods, and populations of neurons described herein, the neuronal cells (such as neurons) are derived from neural stem cells (also known as neural progenitor cells). In some embodiments, the neural stem cells are derived from pluripotent stem cells (such as embryonic stem cells or induced pluripotent stem cells) by methods involving EB formation or co-culture with stromal cell lines. In some embodiments, neural stem cells are derived from pluripotent stem cells by defined serum-free inductions. Human induced pluripotent stem cell-derived neural stem cells (HIP-NSCs) are also commercially available (HIP™ Neural Stem Cells, BC1 line, MTI-GlobalStem). In some embodiments, neurons can be derived from neural stem cells by activation of transcription factors. In some embodiments, neurons can be derived from neural stem cells by activation of ASCL1 and/or NGN2. In some embodiments, an inducible NSC line can be generated from HIP-NSCs that express NGN2 and ASCL1 under an inducible promoter. In some embodiments, a cumate-inducible NGN2/ASCL1 system can be introduced into HIP-NSC line, wherein cumate induction in combination with cell cycle inhibition (PD0332991) in NSC lines can generate homogeneous iPSC-derived neurons. In some embodiments according to any one of the neuronal cell cultures, methods, and populations of neurons described herein, the neurons are derived from mammalian cells (such as mammalian stem cells). In some embodiments, the neurons are derived from primate cells. In some embodiments, the neurons are derived non-human primate (e.g. monkey, baboons, and chimpanzee) cells, mouse cells, rat cells, bovine cells, horse cells, cat cells, dog cells, pig cells, rabbit cells, or goat cells. In some embodiments, the neurons are derived from human cells.

### Application for Neuronal Culture System

### Disease Morphology

The neuronal culture system described herein can be used for studying and validating the disease phenotype and mechanism of action for the neurodegenerative disease such as Alzheimer's disease. In some embodiments, the neuronal culture system demonstrates one or more consistent AD pathologies in neurons upon addition of disease-associated components: synapse loss, pTau induction (hyperphosphorylation) and neuronal loss. In some embodiments, the neuronal culture system reveals a sequence of degeneration events, beginning with synapse loss, axon fragmentation, and dendritic atrophy, followed by p-Tau induction resulting in severe neuronal loss. In some embodiments, upon addition of pro-inflammatory cytokines, the neuronal/microglia co-culture system reveals an increase in microglial cell number, as measured via ionized calcium-binding adapter molecule 1 (IBA1)-positive cell count, suggesting a microgliosis response.

### Drug Screening and Target Discovery

The neuronal culture system described herein can be used for screening (such as including but not limited to discovering, determining, detecting, validating) compounds that provide neuroprotection. The neuronal culture system described herein can be used for discovering (such as including but not limited to discovering, determining, detecting, validating) target pathways that induce disease progression or target pathways that prevent disease progression.

In some embodiments, there is provided a method of screening compounds that increase neuroprotection, comprising: contacting the compound with any one of the neuronal culture systems described herein, and quantifying improvements in neuroprotection. In some embodiments, the improvements in neuroprotection comprises: increase in amounts of one or more of: dendrites, synapses, cell counts, and/or axons in the neuronal culture. In some embodiments, the method comprises quantifying the increase in amounts of one or more of: dendrites, synapses, cell counts, and/or axons in the neuronal culture, wherein: (a) the amount of dendrites is measured by levels of MAP2 in the neuronal culture; (b) the amount of synapses is measured by levels of Synapsin 1 and/or Synapsin 2 in the neuronal culture; (c) the amount of cell counts is measured by levels of CUX2 in the neuronal culture; and/or (d) the amount of axons is measured by levels of beta III tubulin in the neuronal culture.

In some embodiments, a compound is selected for further testing if the level of MAP2 in the neuronal culture is increased by at least about any one of: 10%, 20%, 30%, 40%, 50%, 60%, 70%, 80%, 90%, 100%, 2-fold, 3-fold, 5-fold, 10-fold, 20-fold when compared to a corresponding neuronal culture not contacted with the compound. In some embodiments, a compound is selected for further testing if the level of Synapsin 1 or Synapsin 2 in the neuronal culture is increased by at least about any one of: 10%, 20%, 30%, 40%, 50%, 60%, 70%, 80%. 90%, 100%, 2-fold, 3-fold, 5-fold, 10-fold, 20-fold when compared to a corresponding neuronal culture not contacted with the compound. In some embodiments, a compound is selected for further testing if the level of CUX2 in the neuronal culture is increased by at least about any one of: 10%, 20%, 30%, 40%, 50%, 60%, 70%, 80%, 90%, 100%, 2-fold, 3-fold, 5-fold, 10-fold, 20-fold when compared to a corresponding neuronal culture not contacted with the compound. In some embodiments, a compound is selected for further testing if the level of beta III tubulin in the neuronal culture is increased by at least about any one of: 10%, 20%, 30%, 40%, 50%, 60%, 70%, 80%, 90%, 100%, 2-fold, 3-fold, 5-fold, 10-fold, 20-fold when compared to a corresponding neuronal culture not contacted with the compound.

In some embodiments, the compound is subject to further testing including but not limited to target discovery and analysis of analogs.

In some embodiments, a compound is selected for further testing if: (a) the level of MAP2 in the neuronal culture is increased by ≥30%; (b) the level of Synapsin 1 or Synapsin 2 in the neuronal culture is increased by ≥30%; (c) the level of CUX2 in the neuronal culture is increased by ≥30%; and/or (d) the level of beta III tubulin in the neuronal culture is increased by ≥30%, when compared to a corresponding neuronal culture not contacted with the compound.

In some embodiments, a compound is determined to be neuroprotective if the level of MAP2 in the neuronal culture is increased by at least about any one of: 10%, 20%, 30%, 40%, 50%, 60%, 70%, 80%, 90%, 100%, 2-fold, 3-fold, 5-fold, 10-fold, 20-fold when compared to a corresponding neuronal culture not contacted with the compound. In some embodiments, a compound is determined to be neuroprotective if the level of Synapsin 1 or Synapsin 2 in the neuronal culture is increased by at least about any one of: 10%, 20%, 30%, 40%, 50%, 60%, 70%, 80%, 90%, 100%, 2-fold, 3-fold, 5-fold, 10-fold, 20-fold when compared to a corresponding neuronal culture not contacted with the compound. In some embodiments, a compound determined to be neuroprotective if the level of CUX2 in the neuronal culture is increased by at least about any one of: 10%, 20%, 30%, 40%, 50%, 60%, 70%, 80%, 90%, 100%, 2-fold, 3-fold. 5-fold, 10-fold, 20-fold when compared to a corresponding neuronal culture not contacted with the compound. In some embodiments, a compound is determined to be neuroprotective if the level of beta III tubulin in the neuronal culture is increased by at least about any one of: 10%, 20%. 30%, 40%, 50%, 60%, 70%, 80%, 90%, 100%, 2-fold, 3-fold, 5-fold, 10-fold, 20-fold when compared to a corresponding neuronal culture not contacted with the compound.

In some embodiments, a compound is determined to be neuroprotective if: (a) the level of MAP2 in the neuronal culture is increased by ≥30%; (b) the level of Synapsin 1 or Synapsin 2 in the neuronal culture is increased by ≥30%; (c) the level of CUX2 in the neuronal culture is increased by ≥30%; and/or (d) the level of beta III tubulin in the neuronal culture is increased by ≥30% when compared to a corresponding neuronal culture not contacted with the compound.

### Disease-Associated Components and Neuroprotective Components

In some embodiments according to any one of the neuronal cell cultures, methods, and populations of neurons described herein, the disease-associated component is exogenous to the neurons in the cell culture. In some embodiments, the neuroprotective component is exogenous to the neurons in the cell culture. In some embodiments, the effect of the disease-associated component is dose dependent. In some embodiments, the effect of the neuroprotective component is dose dependent.

### Disease-Associated Component-Soluble Aβ Species

In some embodiments according to any one of the neuronal cell cultures, methods, and populations of neurons described herein, the soluble Aβ species are generated by resuspending lyophilized Aβ monomers (such as AP42 monomers) in PBS and incubating monomers at 4° C. for about any one of: 14, 24, 48, 72 hours then frozen to stop the oligomerization process. In some embodiments, the soluble Aβ species are generated by resuspending lyophilized Aβ monomers (such as AP42 monomers) s in PBS and incubating monomers at 4° C. for about any one of: 7 to 14, 14 to 24, 24 to 48, 48 to 72, or 72 to 96 hours then frozen to stop the oligomerization process. In some embodiments, the soluble Aβ species comprise soluble Aβ oligomers. In some embodiments, the soluble Aβ species comprise soluble Aβ oligomers, Aβ fibrils and/or Aβ monomers. In some embodiments, the soluble AP-induced neurotoxicity is specific to mammalian neurons. In some embodiments, the soluble AP-induced neurotoxicity is specific to primate neurons. In some embodiments, the soluble AP-induced neurotoxicity is specific to human neurons. In some embodiments, the neurons, astrocytes and/or microglia are contacted with about any one of: 0.05, 0.1, 0.2, 0.3, 0.4, 0.5, 0.6, 0.7, 0.8, 0.9, 1, 1.2, 1.4, 16, 1.8 or 2, 3, 4, 5, 6, 7, 8, 9, 10, 12, 14, 16, 18, 20, 30, 50, or 100 μM soluble Aβ species. In some embodiments, the neurons, astrocytes and/or microglia are contacted with about any one of: 0.1, 0.2, 0.25, 0.5, 0.75, 1, 1.25, 1.5, 1.75, 2, 2.5, 3, 3.5, 4, 4.5, 5, 7.5 or 10 μM soluble Aβ species. In some embodiments, the neurons, astrocytes and/or microglia are contacted with soluble Aβ species for about any one of: 1, 2, 3, 4, 5, 6, 7, 8, 9, 10, 11, 12, 13, 14, 15, 16, 17, 18, 19, 20, 21, 24, 28, 30, 35, 40, 50, or 60 days. In some embodiments, the neurons, astrocytes and/or microglia are contacted with soluble Aβ species for about any one of: 2, 5, 7, 14, 21, 28, 30, 40, or 60 days. In some embodiments, the contacting of soluble Aβ species comprises treatment of soluble Aβ species about once a week, twice a week, three times a week, four times a week or once daily. In some embodiments, the soluble Aβ species is a modular component that can be added, removed and/or modified one or more times throughout the duration of screening or disease modeling. In some embodiments, the soluble Aβ species is a tunable component, wherein the concentration of soluble Aβ species can be modified (increased or decreased) one or more times throughout the duration of screening or disease modeling. In some embodiments, the modular and tunable nature of the soluble Aβ species component is facilitated by automated culture media removal and/or automated culture media replenishment in the any one of the automated cell culture systems described herein.

### Disease-Associated Component—Overexpression of Mutant APP

In some embodiments according to any one of the neuronal cell cultures, methods, and populations of neurons described herein, the mutant APP overexpression can be inducible overexpression of mutant APP. In some embodiments, the mutant APP overexpression is a modular component that can be added, removed and/or modified one or more times throughout the duration of screening or disease modeling. In some embodiments, the mutant APP overexpression is a tunable component, wherein the amount of mutant APP overexpression can be modified (increased or decreased) one or more times throughout the duration of screening or disease modeling. In some embodiments, the modular and tunable nature of the mutant APP overexpression component is controlled by modulation of the inducing agent of overexpression, the amount of which is in turn facilitated by automated culture media removal and/or automated culture media replenishment in any one of the automated cell culture systems described herein.

### Disease-Associated Component-Pro-Inflammatory Cytokine

In some embodiments according to any one of the neuronal cell cultures, methods, and populations of neurons described herein, the pro-inflammatory cytokine comprises interferon-gamma (IFNγ), interleukin 1β (IL-1β), lipopolysaccharide (LPS), or any combinations thereof. In some embodiments, the neurons, astrocytes and/or microglia are contacted with about any one of: 1, 2, 5, 10, 20, 30, 40, 50, 60, 70, 80, 90, 100, 150, 200, 250, 300, 350, 400, 450, 500, 600, 700, 800, 900, or 1000 ng/mL IFNγ. In some embodiments, the neurons, astrocytes and/or microglia are contacted with about any one of: 1, 2, 5, 10, 20, 30, 40, 50, 60, 70, 80, 90, 100, 150, 200, 250, 300, 350, 400, 450, 500, 600, 700, 800, 900, or 1000 ng/mL IL-1β. In some embodiments, the neurons, astrocytes and/or microglia are contacted with about any one of: 1, 2, 5, 10, 20, 30, 40, 50, 60, 70, 80, 90, 100, 150, 200, 250, 300, 350, 400, 450, 500, 600, 700, 800, 900, 1000, or 2000 ng/mL LPS. In some embodiments, the neurons, astrocytes and/or microglia are contacted with pro-inflammatory cytokine for about any one of: 1, 2, 3, 4, 5, 6, 7, 8, 9, 10, 11, 12, 13, 14, 15, 16, 17, 18, 19, 20, 21, 24, 28, 30, 35, 40, 50, or 60 days. In some embodiments, the neurons, astrocytes and/or microglia are contacted with pro-inflammatory cytokine for about any one of: 2, 5, 7, 14, 21, 28, 30, 40, or 60 days. In some embodiments, the contacting of pro-inflammatory cytokine about once a week, twice a week, three times a week, four times a week or once daily. In some embodiments, each of the pro-inflammatory cytokines (such as IFNγ, IL-1β, LPS) is a modular component that can be added, removed and/or modified one or more times throughout the duration of screening or disease modeling. In some embodiments, each of the pro-inflammatory cytokines is a tunable component, wherein the concentration of the cytokine can be modified (increased or decreased) one or more times throughout the duration of screening or disease modeling. In some embodiments, the modular and tunable nature of the pro-inflammatory cytokine component is facilitated by automated culture media removal and/or automated culture media replenishment in any one of the automated cell culture systems described herein. In some embodiments, the pro-inflammatory cytokine is a neuroinflammatory cytokine.

### Neuroprotective Component: Anti-Aβ Antibody

In some embodiments according to any one of the neuronal cell cultures, methods, and populations of neurons described herein, the anti-Aβ antibody is Crenezumab. In some embodiments, the neurons, astrocytes and/or microglia are contacted with about any one of: 0.01, 0.05, 0.1, 0.2, 0.3, 0.4, 0.5, 0.6, 0.7, 0.8, 0.9, 1, 1.2, 1.4, 1.6, 1.8 or 2, 3, 4, 5, 6, 7, 9, 10, 12, 14, 16, 18 or 20 μM anti-Aβ antibody. In some embodiments, the neurons, astrocytes and/or microglia are contacted with about any one of: 0.05, 0.1, 0.2, 0.25, 0.5, 0.75, 1, 1.25, 1.5, 1.75, 2, 2.5, 3, 3.5, 4, 4.5, 5, 7.5 or 10 μM anti-Aβ antibody. In some embodiments, the neurons, astrocytes and/or microglia are contacted with anti-Aβ antibody for about any one of: 1, 2, 3, 4, 5, 6, 7, 8, 9, 10, 11, 12, 13, 14, 15, 16, 17, 18, 19, 20, 21, 24, 28, 30, 35, 40, 50, or 60 days. In some embodiments, the neurons, astrocytes and/or microglia are contacted with anti-Aβ antibody for about any one of: 2, 5, 7, 14, 21, 28, 30, 40, or 60 days. In some embodiments, the contacting of anti-A antibody comprises treatment of anti-A antibody about once a week, twice a week, three times a week, four times a week or once daily. In some embodiments, the anti-Aβ antibody is a modular component that can be added, removed and/or modified one or more times throughout the duration of screening or disease modeling. In some embodiments, the anti-Aβ antibody is a tunable component, wherein the concentration of anti-A antibody can be modified (increased or decreased) one or more times throughout the duration of screening or disease modeling. In some embodiments, the modular and tunable nature of the anti-Aβ antibody component is facilitated by automated culture media removal and/or automated culture media replenishment in any one of the automated cell culture systems described herein. Neuroprotective component: DLK inhibitor, GSK3β inhibitor, CDK5 inhibitor, and/or Fyn inhibitor

In some embodiments according to any one of the neuronal cell cultures, methods, and populations of neurons described herein, the neuroprotective component is DLK inhibitor, GSK3M inhibitor, CDK5 inhibitor, JNK inhibitor and/or Fyn kinase inhibitor. In some embodiments, the DLK inhibitor is DLKi, VX-680, GNE-495, PF06260933. In some embodiments, the GSK3β inhibitor is Indirubin-3′-monoxime. In some embodiments, the CDK5 inhibitor is Indirubin-3%-monoxime. In some embodiments, the JNK inhibitor is a JNK1/2/3 inhibitor, optionally wherein the JNK inhibitor is JNK-IN-8. In some embodiments, the Fyn kinase inhibitor is AZD0530. In some embodiments, the neurons, astrocytes and/or microglia are contacted with about any one of: 0.01, 0.05, 0.1, 0.2, 0.3, 0.4, 0.5, 0.6, 0.7, 0.8, 0.9, 1, 1.2, 1.4, 1.6, 1.8 or 2, 3, 4, 5, 6, 7, 8, 9, 10, 12, 14, 16, 18 or 20 μM of one or more of the inhibitors described above. In some embodiments, the neurons, astrocytes and/or microglia are contacted with about any one of: 0.05, 0.1, 0.2, 0.25, 0.5, 0.75, 1, 1.25, 1.5, 1.75, 2, 2.5, 3, 3.5, 4, 4.5, 5, 7.5 or 10 μM of one or more of the inhibitors described above. In some embodiments, the neurons, astrocytes and/or microglia are contacted with of one or more of the inhibitors described above for about any one of: 1, 2, 3, 4, 5, 6, 7, 8, 9, 10, 11, 12, 13, 14, 15, 16, 17, 18, 19, 20, 21, 24, 28, 30, 35, 40, 50, or 60 days. In some embodiments, the neurons, astrocytes and/or microglia are contacted with of one or more of the inhibitors described above for about any one of: 2, 5, 7, 14, 21, 28, 30, 40, or 60 days. In some embodiments, the contacting of the inhibitor comprises treatment of the inhibitor about once a week, twice a week, three times a week, four times a week or once daily. In some embodiments, each of the inhibitors described above is a modular component that can be added, removed and/or modified one or more times throughout the duration of screening or disease modeling. In some embodiments, each of the inhibitors described above is a tunable component, wherein the concentration of each inhibitor can be modified (increased or decreased) one or more times throughout the duration of screening or disease modeling. In some embodiments, the modular and tunable nature of each of the inhibitors described above is facilitated by automated culture media removal and/or automated culture media replenishment in any one of the automated cell culture systems described herein.

### Neuroprotective Component: Microglia

In some embodiments according to any one of the neuronal cell cultures, methods, and populations of neurons described herein, the microglia are derived from PSCs (such as iPSC or ESCs) according to published protocol, such as described in Abud et al., 2017. In some embodiments, the method of generating microglia comprises treating iPSCs with BMP, FGF and Activin for 2-4 days to induce mesoderm fate, then treating with VEGF and supportive hematopoietic cytokines for 6-10 days to generate hematopoietic progenitors (HPCs), wherein HPCs are seeded onto Matrigel-coated flasks, and further treated with IL-34, IDE1 (TGF β1 agonist), and M-CSF for 3-4 weeks to differentiate into microglia. In some embodiments, neurons and/or astrocytes contacted (such as co-cultured) with microglia for about any one of: 1, 2, 3, 4, 5, 6, 7, 8, 9, 10, 11, 12, 13, 14, 15, 16, 17, 18, 19, 20, 21, 24, 28, 30, 35, 40, 50, or 60 days. In some embodiments, neurons and/or astrocytes contacted (such as co-cultured) with microglia for about any one of: 2, 5, 7, 14, 21, 28, 30, 40, or 60 days. In some embodiments, the contacting of microglia comprises seeding microglial cells about once a month, once every three weeks, once every two weeks, once every 10 days once a week, twice a week, three times a week, four times a week or once daily. In some embodiments, the microglia is a modular component that can be added and/or modified one or more times throughout the duration of screening or disease modeling. In some embodiments, the microglia is a tunable component, wherein the concentration of microglia can be modified (such as increased) one or more times throughout the duration of screening or disease modeling. In some embodiments, the modular and tunable nature of the microglia component is facilitated by cell seeding using automated culture media removal and/or automated culture media replenishment in the any one of the automated cell culture systems described herein.

### Systems and Kits

In some aspects, the invention provides an integrated system comprising one or more of the automated cell culture system, PSC-derived NSC lines, differentiated neurons, neuronal culture system models, disease-associated components and/or neuroprotective components disclosed herein. The system can include any embodiment described for the methods disclosed above, including methods of generating fully differentiated neurons, methods of modeling AD and/or methods of drug screening and target discovery described herein. In some embodiments, the parameters of the differentiation, maturation, disease-associated components and/or neuroprotective components, such as concentrations and intervals of component administration, duration of differentiation and maturation, and cell culture media (e.g., osmolarity, salt concentration, serum content of media, cell concentration, pH, etc.) are optimized for modeling of AD and drug screening.

Also provided are kits or articles of manufacture for use in modeling AD. In some embodiments, the kit comprises an automated cell culture system, PSC-derived NSC lines, differentiated neurons, neuronal culture system models, disease-associated components and/or neuroprotective components disclosed herein. In some embodiments, the kits comprise the compositions described herein (e.g. PSC-derived NSC lines, differentiated neurons, disease-associated components and/or neuroprotective components) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The invention also provides kits comprising components of the methods described herein and may further comprise instructions for performing said methods of modeling neurodegenerative diseases or drug screening. The kits described herein may further include other materials, including other buffers, diluents, filters, pipet tips, tissue culture plates, automated culture systems and package inserts with instructions for performing any methods described herein; e.g., methods of modeling neurodegenerative diseases or drug screening.

### EXEMPLARY EMBODIMENTS

Embodiment 1. An automated cell culture system for facilitating neuronal differentiation and/or promoting long-term neuronal growth, wherein the automated cell culture system comprises one or more rounds of automated culture media replacements; and wherein the automated cell culture system sustains differentiation, maturation and/or growth of neuronal cells for at least about any one of: 30, 60, 80, 90, 120, or 150 days.

Embodiment 2. The automated cell culture system of embodiment 1, wherein the automated culture media replacement comprises automated culture media aspiration and automated culture media replenishment; and/or wherein the cell culture system comprises one or more 96-well plates; or one or more 384-well plates.

Embodiment 3. The automated cell culture system of embodiment 2, wherein the automated culture media aspiration comprises aspiration with a pipet tip, wherein: the distal end of the pipet tip is at about 1 mm above the bottom surface of the well before, during and/or after the aspiration.

Embodiment 4. The automated cell culture system of embodiment 2 or 3, wherein the automated culture media aspiration comprises aspiration with a pipet tip, wherein: the pipet tip is at an angle of about 90° to the bottom surface of the well before, during and/or after the aspiration.

Embodiment 5. The automated cell culture system of any one of embodiments 2-4, wherein the automated culture media aspiration comprises aspiration with a pipet tip, wherein: the pipet tip has a displacement of no more than 0.1 mm from the center of the well before, during and/or after the aspiration; optionally wherein the pipet tip is at the center of the well before, during and/or after the aspiration (no displacement).

Embodiment 6. The automated cell culture system of any one of embodiments 2-5, wherein the automated culture media aspiration comprises aspiration with a pipet tip, wherein: (a) the speed of media aspiration is no more than about 7.5 μl/s; and/or (b) the start of media aspiration is about 200 ms subsequent to the pipet tip being placed 1 mm above the bottom surface of the well.

Embodiment 7. The automated cell culture system of any one of embodiments 2-6, wherein the automated culture media aspiration comprises aspiration with a pipet tip, wherein: (a) the pipet tip is inserted into the well at a speed of about 5 mm/s prior to aspiration; and/or (b) the pipet tip is withdrawn from the well at a speed of about 5 mm/s after the aspiration.

Embodiment 8. The automated cell culture system of any one of embodiments 2-7, wherein the cell culture system comprises a 384-well plate; further wherein the automated cell culture system comprises automated discarding of a used rack of 384-pipet tips and automated engagement of a new rack of 384-pipet tips subsequent to each round of media aspiration.

Embodiment 9. The automated cell culture system of any one of embodiments 2-7, wherein the cell culture system comprises one or more batches of 384-well plates, wherein each batch comprises up to twenty-five 384-well plates arranged in 5 columns and 5 rows; further wherein: the automated cell culture system comprises automated discarding of up to 25 corresponding used racks of 384-pipet tips and automated engagement of up to 25 corresponding new racks of 384-pipet tips subsequent to each round of media aspiration.

Embodiment 10. The automated cell culture system of any one of embodiments 2-9, wherein the automated culture media replenishment comprises dispensing media with a pipet tip, wherein: (a) the distal end of the pipet tip is at about 1 mm above the bottom surface of the well before the dispensing; and/or (b) the pipet tip is withdrawn from the well at a speed of about 1 mm/s during the dispensing.

Embodiment 11. The automated cell culture system of any one of embodiments 2-10, wherein the automated culture media replenishment comprises dispensing media with a pipet tip, wherein: the pipet tip is at an angle of about 90° to the bottom surface of the well before and/or during the dispensing.

Embodiment 12. The automated cell culture system of any one of embodiments 2-11, wherein the automated culture media replenishment comprises dispensing media with a pipet tip, wherein: the pipet tip has a displacement of no more than 0.1 mm from the center of the well before, and/or during the dispensing, optionally wherein the pipet tip is at the center of the well before, and/or during the dispensing (no displacement).

Embodiment 13. The automated cell culture system of any one of embodiments 2-12, wherein the cell culture system comprises a 384-well tissue plate; wherein the automated culture media replenishment comprises dispensing media with a pipet tip, wherein: (a) the pipet tip is displaced to contact a first side of the well 1 mm from the center in a first direction, at a height of about 12.40 mm above the bottom of the well at a speed of about 100 mm/s; and/or (b) the pipet tip is displaced to contact a second side of the well 1 mm from the center in a second direction, at a height of about 12.40 mm above the bottom of the well at a speed of about 100 mm/s, optionally wherein the first direction is at an angle of about 1800 to the second direction.

Embodiment 14. The automated cell culture system of any one of embodiments 2-13, wherein the automated culture media replenishment comprises dispensing media with a pipet tip, wherein:

(a) the speed of media dispensing is no more than about 1.5 μl/s;
(b) the acceleration of media dispensing is about 500 μl/s²;
(c) the deceleration of media dispensing is about 500 μl/s²; and/or
(d) the start of media dispensing is about 200 ms subsequent to the pipet tip being placed 1 mm above the bottom surface of the well.

Embodiment 15. The automated cell culture system of any one of embodiments 2-14, wherein the automated culture media replenishment comprises dispensing media with a pipet tip, wherein:

(a) the pipet tip is inserted into the well at a speed of about 5 mm/s prior to dispensing; and/or
(b) the pipet tip is withdrawn from the well at a speed of about 5 mm/s after the dispensing.

Embodiment 16. The automated cell culture system of any one of embodiments 2-15, wherein the cell culture system comprises a 384-well plate; further wherein the automated cell culture system comprises automated discarding of a used rack of 384-pipet tips and automated engagement of a new rack of 384-pipet tips subsequent to each round of media dispensing.

Embodiment 17. The automated cell culture system of any one of embodiments 2-16, wherein the cell culture system comprises one or more batches of 384-well plates, wherein each batch comprises up to twenty-five 384-well plates arranged in 5 columns and 5 rows; further wherein the automated cell culture system comprises automated discarding of up to 25 corresponding used racks of 384-pipet tips and automated engagement of up to 25 corresponding new racks of 384-pipet tips subsequent to each round of media dispensing.

Embodiment 18. The automated cell culture system of any one of any one of embodiments 1-17, wherein the time interval between two rounds of culture media replacements is about any one of: 1, 2, 3, 4, 5, 6, 7, 8, 9, or 10 days.

Embodiment 19. The automated cell culture system of any one of any one of embodiments 1-18, wherein the time interval between two rounds of culture media replacements is about 3 or 4 days.

Embodiment 20. The automated cell culture system of any one of embodiments 1-19, wherein about any one of: 30%, 40%, 50%, 60%, 70%, or 80% of culture media is replaced in one or more rounds of culture media replacement.

Embodiment 21. The automated cell culture system of any one of embodiments 1-19, wherein about any one of: 30%, 40%, 50%, 60%, 70%, or 80% of culture media is replaced in each round of culture media replacement.

Embodiment 22. The automated cell culture system of any one of embodiments 1-21, wherein about 50% of culture media is replaced in one or more rounds of culture media replacement.

Embodiment 23. The automated cell culture system of any one of embodiments 1-21, wherein about 50% of culture media is replaced in each round of culture media replacement.

Embodiment 24. A method of generating homogenous and terminally differentiated neurons from pluripotent stem cells, comprising:

(a) generating a pluripotent stem cell- (PSC-) derived neural stem cell (NSC) line expressing NGN2, and ASCL1 under an inducible system;
(b) culturing the NSC line under conditions to induce the expression of NGN2 and ASCL1, in combination with a cell cycle inhibitor for at least about 7 days, thereby generating PSC-derived neurons;
(c) replating the PSC-derived neurons in presence of primary human astrocytes;
(d) differentiating and maturing the PSC-derived neurons for at least about 60 to about 90 days in an automated cell culture system.

Embodiment 25. The method of embodiment 24, wherein the step of differentiating and maturing the PSC-derived neurons comprises one or more rounds of automated culture media replacements using an automated cell culture system; and wherein the automated cell culture system sustains differentiation, maturation and/or growth of neuronal cells for at least about any one of: 30, 60, 80, 90, 120, or 150 days.

Embodiment 26. The method of embodiment 25, wherein the automated culture media replacement comprises automated culture media aspiration and automated culture media replenishment; and/or wherein the cell culture system comprises one or more tissue culture plates.

Embodiment 27. The method of embodiment 26, wherein the automated culture media aspiration comprises aspiration with a pipet tip, wherein:

(a) the distal end of the pipet tip is at about 1 mm above the bottom surface of the well before, during and/or after the aspiration;
(b) the pipet tip is at an angle of about 90° to the bottom surface of the well before, during and/or after the aspiration;
(c) the pipet tip has a displacement of no more than 0.1 mm from the center of the well before, during and/or after the aspiration; optionally wherein the pipet tip is at the center of the well before, during and/or after the aspiration (no displacement);
(d) the speed of media aspiration is no more than about 7.5 μl/s;
(e) the start of media aspiration is about 200 ms subsequent to the pipet tip being placed 1 mm above the bottom surface of the well;
(f) the pipet tip is inserted into the well at a speed of about 5 mm/s prior to aspiration; and/or
(g) the pipet tip is withdrawn from the well at a speed of about 5 mm/s after aspiration.

Embodiment 28. The method of embodiment 26 or 27, wherein the automated culture media replenishment comprises dispensing media with a pipet tip, wherein:

(a) the distal end of the pipet tip is at about 1 mm above the bottom surface of the well before the dispensing;
(b) the distal end of the pipet tip is withdrawn from the well at about 1 mm/s during the dispensing;
(c) the pipet tip is at an angle of about 90° to the bottom surface of the well before and/or during the dispensing;
(d) the pipet tip has a displacement of no more than 0.1 mm from the center of the well before, and/or during the dispensing, optionally wherein the pipet tip is at the center of the well before, and/or during the dispensing (no displacement);
(e) the pipet tip is displaced to contact a first side of the well 1 mm from the center in a first direction, at a height of about 12.40 mm above the bottom of the well at a speed of about 100 mm/s;
(f) the pipet tip is displaced to contact a second side of the well 1 mm from the center in a second direction, at a height of about 12.40 mm above the bottom of the well at a speed of about 100 mm/s, optionally wherein the first direction is at an angle of about 1800 to the second direction;
(g) the speed of media dispensing is no more than about 1.5 μl/s;
(h) the acceleration of media dispensing is about 500 μl/s²;
(i) the deceleration of media dispensing is about 500 μl/s²;
(j) the start of media dispensing is about 200 ms subsequent to the pipet tip being placed 1 mm above the bottom surface of the well;
(k) the pipet tip is inserted into the well at a speed of about 5 mm/s prior to dispensing; and/or
(l) the pipet tip is withdrawn from the well at a speed of about 5 mm/s after dispensing.

Embodiment 29. The method of any one of embodiments 26-28, wherein the cell culture system comprises a 384-well plate; further wherein:

(a) the automated cell culture system comprises automated discarding of a used rack of 384-pipet tips and automated engagement of a new rack of 384-pipet tips subsequent to each round of media aspiration; and/or
(b) the automated cell culture system comprises automated discarding of a used rack of 384-pipet tips and automated engagement of a new rack of 384-pipet tips subsequent to each round of media dispensing.

Embodiment 30. The method of any one of embodiments 26-29, wherein the cell culture system comprises one or more batches of 384-well plates, wherein each batch comprises up to twenty-five 384-well plates arranged in 5 columns and 5 rows; further wherein:

(a) the automated cell culture system comprises automated discarding of up to 25 corresponding used racks of 384-pipet tips and automated engagement of up to 25 corresponding new racks of 384-pipet tips subsequent to each round of media aspiration; and/or
(b) the automated cell culture system comprises automated discarding of up to 25 corresponding used racks of 384-pipet tips and automated engagement of up to 25 corresponding new racks of 384-pipet tips subsequent to each round of media dispensing.

Embodiment 31. The method of any one of embodiments 26-30, wherein:

(a) the time period between two rounds of culture media replacements is about any one of: 1, 2, 3, 4, 5, 6, 7, 8, 9, or 10 days; and/or
(b) about any one of: 30%, 40%, 50%, 60%, 70%, or 80% of culture media is replaced in one or more rounds of culture media replacement.

Embodiment 32. The method of any one of embodiments 26-31, wherein:

(a) the time period between two rounds of culture media replacements is about 3 or 4 days; and/or
(b) about 50% of culture media is replaced in one or more rounds of culture media replacement.

Embodiment 33. A homogenous population of terminally differentiated neurons derived from pluripotent stem cells, wherein at least 95% of the neurons express: Map2; Synapsin 1 and/or Synapsin 2; and beta-III tubulin.

Embodiment 34. A homogenous population of terminally differentiated neurons derived from pluripotent stem cells, wherein:

(a) at least 95% of the neurons express one or more pre-synaptic markers selected from vGLUT2, Synapsin 1, and Synapsin 2; and/or
(b) at least 95% of the neurons express one or more post-synaptic markers selected from: PSD95, SHANK, PanSHANK, GluR1, GluR2, PanSAPAP, and NR1; and/or
(c) at least 100 postsynaptic endings of a neuron overlap with presynaptic endings of other neurons and/or at least 100 presynaptic endings of the neuron overlap with postsynaptic endings of other neurons.

Embodiment 35. The population of embodiment 34, wherein at least 95% of the neurons express: two or more pre-synaptic markers selected from: vGLUT2, Synapsin 1, and Synapsin 2; and/or two or more post-synaptic markers selected from: PSD95, SHANK, PanSHANK, GluR1, GluR2, PanSAPAP, and NR1.

Embodiment 36. The population of any one of embodiments 33-35, wherein at least 95% of the neurons express one or more upper-layer cortical neuron markers, optionally wherein no more than 5% of the neurons express one or more lower layer cortical neuron markers

Embodiment 37. The population of any one of embodiments 33-36, wherein at least 95% of neurons express CUX2, optionally wherein no more than 5% of the neurons express CTIP2 or SATB2.

Embodiment 38. The population of any one of embodiments 33-37, wherein the process of deriving terminally differentiated neurons from pluripotent stem cells comprises:

(a) generating a pluripotent stem cell- (PSC-) derived neural stem cell (NSC) line expressing NGN2, and ASCL1 under an inducible system;
(b) culturing the NSC line under conditions to express NGN2 and ASCL1, in combination with cell cycle inhibitor for at least about 7 days, thereby generating PSC-derived neurons;
(c) replating the PSC-derived neurons in presence of primary human astrocytes;
(d) differentiating and maturing the PSC-derived neurons for at least about 60 to about 90 days in an automated cell culture system.

Embodiment 39. The population of embodiment 38, wherein the neurons express representative markers for dendrites, cell bodies, axons and synapses in highly replicable manner.

Embodiment 40. The population of embodiment 39, wherein the expressions of dendritic marker MAP2, cell body marker CUX2, axon marker Tau, and synapse marker Synapsin 1/2 in neurons are highly replicable across replicate experiments, wherein the z-factor for each of MAP2, CUX2, Tau and Synapsin 1/2 is at least 0.4.

Embodiment 41. The population of any one of embodiments 38-40, wherein the step of differentiating and maturing the PSC-derived neurons comprises one or more rounds of automated culture media replacements; and wherein the automated cell culture system sustains differentiation, maturation and/or growth of neuronal cells for at least about any one of: 30, 60, 80, 90, 120, or 150 days.

Embodiment 42. The population of embodiment 41, wherein the automated culture media replacement comprises automated culture media aspiration and automated culture media replenishment; and/or wherein the cell culture system comprises one or more 384-well plates.

Embodiment 43. The population of embodiment 42, wherein the automated culture media aspiration comprises aspiration with a pipet tip, wherein:

(a) the distal end of the pipet tip is at about 1 mm above the bottom surface of the well before, during and/or after the aspiration;
(b) the pipet tip is at an angle of about 90° to the bottom surface of the well before, during and/or after the aspiration;
(c) the pipet tip has a displacement of no more than 0.1 mm from the center of the well before, during and/or after the aspiration; optionally wherein the pipet tip is at the center of the well before, during and/or after the aspiration (no displacement);
(d) the speed of media aspiration is no more than about 7.5 μl/s;
(e) the start of media aspiration is about 200 ms subsequent to the pipet tip being placed 1 mm above the bottom surface of the well;
(f) the pipet tip is inserted into the well at a speed of about 5 mm/s prior to aspiration; and/or
(g) the pipet tip is withdrawn from the well at a speed of about 5 mm/s after aspiration.

Embodiment 44. The population of embodiment 42 or 43, wherein the automated culture media replenishment comprises dispensing media with a pipet tip, wherein:

(a) the distal end of the pipet tip is at about 1 mm above the bottom surface of the well before the dispensing;
(b) the distal end of the pipet tip is withdrawn from the well at about 1 mm/s during the dispensing;
(c) the pipet tip is at an angle of about 90° to the bottom surface of the well before and/or during the dispensing;
(d) the pipet tip has a displacement of no more than 0.1 mm from the center of the well before, and/or during the dispensing, optionally wherein the pipet tip is at the center of the well before, and/or during the dispensing (no displacement);
(e) the pipet tip is displaced to contact a first side of the well 1 mm from the center in a first direction, at a height of about 12.40 mm above the bottom of the well at a speed of about 100 mm/s;
(f) the pipet tip is displaced to contact a second side of the well 1 mm from the center in a second direction, at a height of about 12.40 mm above the bottom of the well at a speed of about 100 mm/s, optionally wherein the first direction is at an angle of about 1800 to the second direction;
(g) the speed of media dispensing is no more than about 1.5 μl/s;
(h) the acceleration of media dispensing is about 500 μl/s²;
(i) the deceleration of media dispensing is about 500 μl/s²;
(j) the start of media dispensing is about 200 ms subsequent to the pipet tip being placed 1 mm above the bottom surface of the well;
(k) the pipet tip is inserted into the well at a speed of about 5 mm/s prior to dispensing; and/or
(l) the pipet tip is withdrawn from the well at a speed of about 5 mm/s after dispensing.

Embodiment 45. The population of any one of embodiments 42-44, wherein the cell culture system comprises a 384-well plate; further wherein:

(a) the automated cell culture system comprises automated discarding of a used rack of 384-pipet tips and automated engagement of a new rack of 384-pipet tips subsequent to each round of media aspiration; and/or
(b) the automated cell culture system comprises automated discarding of a used rack of 384-pipet tips and automated engagement of a new rack of 384-pipet tips subsequent to each round of media dispensing.

Embodiment 46. The population of any one of embodiments 42-45, wherein the cell culture system comprises one or more batches of 384-well plates, wherein each batch comprises up to twenty-five 384-well plates arranged in 5 columns and 5 rows; further wherein:

(a) the automated cell culture system comprises automated discarding of up to 25 corresponding used racks of 384-pipet tips and automated engagement of up to 25 corresponding new racks of 384-pipet tips subsequent to each round of media aspiration; and/or
(b) the automated cell culture system comprises automated discarding of up to 25 corresponding used racks of 384-pipet tips and automated engagement of up to 25 corresponding new racks of 384-pipet tips subsequent to each round of media dispensing.

Embodiment 47. The population of any one of embodiments 42-46, wherein:

(a) the time period between two rounds of culture media replacements is about any one of: 1, 2, 3, 4, 5, 6, 7, 8, 9, or 10 days; and/or
(b) about any one of: 30%, 40%, 50%, 60%, 70%, or 80% of culture media is replaced in one or more rounds of culture media replacement.

Embodiment 48. The population of any one of embodiments 42-47, wherein:

(a) the time period between two rounds of culture media replacements is about 3 or 4 days; and/or
(b) about 50% of culture media is replaced in one or more rounds of culture media replacement.

Embodiment 49. A pluripotent stem cell-derived neuronal culture system for use in modeling neurodegenerative diseases, wherein the culture system comprises substantially defined culture media and wherein the culture system is amenable to modular and tunable inputs of: one or more disease-associated components and/or one or more neuroprotective components.

Embodiment 50. The neuronal culture system of embodiment 49, wherein the neurodegenerative disease is Alzheimer's disease, wherein:

(a) the disease-associated components comprises soluble Aβ species;
(b) the disease-associated component comprises overexpression of mutant APP, optionally wherein the disease-associated component comprises inducible overexpression of mutant APP;
(c) the disease-associated component comprises pro-inflammatory cytokine;
(d) the neuroprotective component comprises anti-Aβ antibody;
(e) the neuroprotective component comprises DLK inhibitor, GSK3β inhibitor, CDK5 inhibitor, and/or Fyn kinase inhibitor; and/or
(f) the neuroprotective component comprises microglia.

Embodiment 51. The neuronal culture system of embodiment 49 or 50, wherein the system does not comprise matrigel.

Embodiment 52. The neuronal culture system of any one of embodiments 49-51, wherein the system comprises completely defined culture media and/or matrices.

Embodiment 53. The culture system of any one of embodiments 50-52, wherein the soluble Aβ species comprises soluble Aβ oligomers and/or soluble Aβ fibrils.

Embodiment 54 The neuronal culture system of any one of embodiments 50-53, wherein the neuronal culture system comprises the disease-associated component comprising soluble Aβ species, wherein: Tau protein in the neuronal culture is hyperphosphorylated in one or more of S396/404, S217, S235, S400/T403/S404, and T181 residues.

Embodiment 55. The neuronal culture system of any one of embodiments 50-54, wherein the culture system comprises the one or more disease-associated components comprising soluble Aβ species, wherein:

the neuronal culture system displays increased neuronal toxicity as compared to a corresponding neuronal culture system not comprising the soluble Aβ species.

Embodiment 56. The neuronal culture system of any one of embodiments 50-55, wherein the neuronal culture system comprises the disease-associated component comprising soluble Aβ species, wherein:

the culture system displays a decrease of MAP2-positive neurons as compared to a corresponding neuronal culture system not comprising the soluble Aβ species.

Embodiment 57. The neuronal culture system of any one of embodiments 50-56, wherein the neuronal culture system comprises the disease-associated component comprising soluble Aβ species, wherein:

the culture system displays a decrease of synapsin-positive neurons as compared to neuronal culture system not comprising the soluble Aβ species.

Embodiment 58. The neuronal culture system of any one of embodiments 50-57, wherein the neuronal culture system comprises the disease-associated component comprising soluble Aβ species, wherein:

the neuronal culture system displays an increase in Tau phosphorylation in neurons as compared to a neuronal culture system not comprising the soluble Aβ species, wherein the concentration of Aβ is no less than a first concentration;
the neuronal culture system displays a decrease of synapsin-positive neurons as compared to a neuronal culture system not comprising the soluble Aβ species, wherein the concentration of Aβ is no less than a second concentration;
the culture system displays a decrease of CUX2-positive neurons as compared to neuronal culture system not comprising the soluble Aβ species, wherein the concentration of Aβ is no less than a third concentration; and
the culture system displays a decrease of MAP2-positive neurons as compared to neuronal culture system not comprising the soluble Aβ species, wherein Aβ is at no less than a forth concentration.

Embodiment 59. The neuronal culture system of embodiment 58, wherein:

the first concentration is higher than the second, third and fourth concentrations; and/or the second concentration is higher than the third and fourth concentrations; and/or the third concentration is higher than the fourth concentration.

Embodiment 60. The neuronal culture system of embodiment 59, wherein the first concentration is about 5 μM, the second concentration is about 2.5 μM, the third concentration is about 1.25 μM and the fourth concentration is about 0.3 μM.

Embodiment 61. The neuronal culture system of any one of embodiments 50-53, wherein the neuronal culture system comprises the disease-associated component comprising soluble Aβ species, wherein:

the neuronal culture system further comprises astrocytes in co-culture, wherein the astrocytes exhibit increased GFAP expression and/or the astrocytes exhibit increased GFAP fragmentation as compared to astrocytes co-cultured in a neuronal culture system not comprising the soluble Aβ species.

Embodiment 62. The neuronal culture system of any one of embodiments 50-53, wherein the neuronal culture system comprises the disease-associated component comprising soluble Aβ species, wherein:

the neuronal culture system exhibits Methoxy X04-positive Aβ plaques or plaque-like structures.

Embodiment 63. The neuronal culture system of embodiment 62, wherein the neuronal culture system exhibits neuritic dystrophy.

Embodiment 64. The neuronal culture system of embodiment 62, wherein at least a subset of the Methoxy X04-positive Aβ plaques or plaque-like structures are surrounded by neurites, optionally wherein the neurites are marked by neurofilament heavy chain (NFL-H) axonal swelling and/or phosphorylated Tau (S235) positive blebbings, further optionally wherein the neurites are dystrophic.

Embodiment 65. The neuronal culture system of embodiment 64, wherein the plaques or plaque-like structures surrounded by neurites exhibit: ApoE expression localized in the amyloid plaques and/or APP in the membranes of the neurites

Embodiment 66. The neuronal culture system of any one of embodiments 50-53, wherein the culture system comprises: the disease-associated component comprising soluble Aβ species, the disease-associated component comprising neuroinflammatory cytokine, and the neuroprotective component comprising microglia.

Embodiment 67. The neuronal culture system of embodiment 50 or 66, wherein the microglia is iPSC-derived microglia and expresses one or more of: TREM2, TMEM 119, CXCR1, P2RY12, PU.1, MERTK, CD33, CD64, CD32 and IBA-1.

Embodiment 68. The neuronal culture system of any one of embodiments 66-67, wherein the neuronal culture system comprising (1) soluble Aβ species, and (2) microglia exhibits decreased neuronal toxicity as compared to a corresponding neuronal culture system not comprising microglia.

Embodiment 69. The neuronal culture system of any one of embodiments 66-68, wherein the neuronal culture system comprising (1) soluble Aβ species, and (2) microglia exhibits increased microglial-Aβ plaque association and/or increased Aβ plaque formation as compared to a corresponding neuronal culture system not comprising microglia.

Embodiment 70. The neuronal culture system of any one of embodiments 66-69, wherein the neuronal culture system comprising (1) soluble Aβ species, (2) neuroinflammatory cytokine and (3) microglia exhibits less than 10% change in neuronal toxicity as compared to a corresponding neuronal culture system not comprising microglia.

Embodiment 71. The neuronal culture system of any one of embodiments 66-70, wherein the neuronal culture system comprising (1) soluble Aβ species, (2) neuroinflammatory cytokine and (3) microglia exhibits increased microglial-sAβ plaque association and/or increased sAβ plaque formation as compared to a corresponding neuronal culture system not comprising microglia.

Embodiment 72. The neuronal culture system of any one of embodiments 50-53, wherein the neuronal culture system comprises the disease-associated component comprising (1) the disease-associated component comprising soluble Aβ species, and (2) the neuroprotective component comprising microglia.

Embodiment 73. The neuronal culture system of any one of embodiments 49-72, wherein the neurons exhibit one or more of DLK, GSK3, CDK5, and Fyn kinase signaling.

Embodiment 74. The neuronal culture system of any one of embodiments 49-73, wherein the neuronal culture comprises homogenous and terminally differentiated neurons from pluripotent stem cells, wherein the homogenous and terminally differentiated neurons from pluripotent stem cells are generated in a process comprising the steps of:

(a) generating a pluripotent stem cell- (PSC-) derived neural stem cell (NSC) line expressing NGN2, and ASCL1 under an inducible system.
(b) culturing the NSC line under conditions to induce the expression of NGN2 and ASCL1, in combination with a cell cycle inhibitor for at least about 7 days, thereby generating PSC-derived neurons;
(c) replating the PSC-derived neurons in presence of primary human astrocytes;
(d) differentiating and maturing the PSC-derived neurons for at least about 60 to about 90 days in an automated cell culture system.

Embodiment 75. The neuronal culture system of embodiment 76, wherein the step of differentiating and maturing the PSC-derived neurons comprises one or more rounds of automated culture media replacements; and wherein the automated cell culture system sustains differentiation, maturation and/or growth of neuronal cells for at least about any one of: 30, 60, 80, 90, 120, or 150 days.

Embodiment 76. The neuronal culture system of embodiment 74 or 75, wherein the automated culture media replacement comprises automated culture media aspiration and automated culture media replenishment; and/or wherein the cell culture system comprises one or more 384-well plates.

Embodiment 77. The neuronal culture system of embodiment 76, wherein the automated culture media aspiration comprises aspiration with a pipet tip, wherein:

(a) the distal end of the pipet tip is at about 1 mm above the bottom surface of the well before, during and/or after the aspiration;
(b) the pipet tip is at an angle of about 90° to the bottom surface of the well before, during and/or after the aspiration;
(c) the pipet tip has a displacement of no more than 0.1 mm from the center of the well before, during and/or after the aspiration; optionally wherein the pipet tip is at the center of the well before, during and/or after the aspiration (no displacement);
(d) the speed of media aspiration is no more than about 7.5 μl/s;
(e) the start of media aspiration is about 200 ms subsequent to the pipet tip being placed 1 mm above the bottom surface of the well
(f) the pipet tip is inserted into the well at a speed of about 5 mm/s prior to aspiration; and/or
(g) the pipet tip is withdrawn from the well at a speed of about 5 mm/s after aspiration.

Embodiment 78. The neuronal culture system of embodiment 76 or 77, wherein the automated culture media replenishment comprises dispensing media with a pipet tip, wherein:

(a) the distal end of the pipet tip is at about 1 mm above the bottom surface of the well before the dispensing;
(b) the distal end of the pipet tip is withdrawn from the well at about 1 mm/s during the dispensing;
(c) the pipet tip is at an angle of about 90° to the bottom surface of the well before and/or during the dispensing;
(d) the pipet tip has a displacement of no more than 0.1 mm from the center of the well before, and/or during the dispensing, optionally wherein the pipet tip is at the center of the well before, and/or during the dispensing (no displacement);
(e) the pipet tip is displaced to contact a first side of the well about 1 mm from the center in a first direction, at a height of about 12.40 mm above the bottom of the well at a speed of about 100 mm/s;
(f) the pipet tip is displaced to contact a second side of the well about 1 mm from the center in a second direction, at a height of about 12.40 mm above the bottom of the well at a speed of about 100 mm/s, optionally wherein the first direction is at an angle of about 1800 to the second direction;
(g) the speed of media dispensing is no more than about 1.5 μl/s;
(h) the acceleration of media dispensing is about 500 μl/s²;
(i) the deceleration of media dispensing is about 500 μl/s²;
(j) the start of media dispensing is about 200 ms subsequent to the pipet tip being placed 1 mm above the bottom surface of the well;
(k) the pipet tip is inserted into the well at a speed of about 5 mm/s prior to dispensing; and/or
(l) the pipet tip is withdrawn from the well at a speed of about 5 mm/s after dispensing.

Embodiment 79. The neuronal culture system of any one of embodiments 76-78, wherein the cell culture system comprises a 384-well plate; further wherein:

(a) the automated cell culture system comprises automated discarding of a used rack of 384-pipet tips and automated engagement of a new rack of 384-pipet tips subsequent to each round of media aspiration; and/or
(b) the automated cell culture system comprises automated discarding of a used rack of 384-pipet tips and automated engagement of a new rack of 384-pipet tips subsequent to each round of media dispensing.

Embodiment 80. The neuronal culture system of any one of embodiments 76-79, wherein the cell culture system comprises one or more batches of 384-well plates, wherein each batch comprises up to twenty-five 384-well plates arranged in 5 columns and 5 rows; further wherein:

(a) the automated cell culture system comprises automated discarding of up to 25 corresponding used racks of 384-pipet tips and automated engagement of up to 25 corresponding new racks of 384-pipet tips subsequent to each round of media aspiration; and/or
(b) the automated cell culture system comprises automated discarding of up to 25 corresponding used racks of 384-pipet tips and automated engagement of up to 25 corresponding new racks of 384-pipet tips subsequent to each round of media dispensing.

Embodiment 81. The neuronal culture system of any one of embodiments 76-80, wherein:

(a) the time period between two rounds of culture media replacements is about any one of: 1, 2, 3, 4, 5, 6, 7, 8, 9, or 10 days; and/or
(b) about any one of: 30%, 40%, 50%, 60%, 70%, or 80% of culture media is replaced in one or more rounds of culture media replacement.

Embodiment 82. The neuronal culture system of any one of embodiments 76-81, wherein:

(a) the time period between two rounds of culture media replacements is about 3 or 4 days; and/or
(b) about 50% of culture media is replaced in one or more rounds of culture media replacement.

Embodiment 83. A method of screening compounds that increase neuroprotection, comprising: contacting the compound with the neuronal culture in the neuronal culture system of any one of embodiments 50-82, and quantifying improvements in neuroprotection.

Embodiment 84. The method of embodiment 83, wherein the improvements in neuroprotection comprises: increase in amounts of one or more of: dendrites, synapses, cell counts, and/or axons in the neuronal culture.

Embodiment 85. The method of embodiment 84, wherein the method comprises quantifying the increase in amounts of one or more of: dendrites, synapses, cell counts, and/or axons in the neuronal culture, wherein:

(a) the amount of dendrites is measured by levels of MAP2 in the neuronal culture;
(b) the amount of synapses is measured by levels of Synapsin 1 and/or Synapsin 2 in the neuronal culture;
(c) the amount of cell counts is measured by levels of CUX2 in the neuronal culture; and/or
(d) the amount of axons is measured by levels of beta III tubulin in the neuronal culture.

Embodiment 86. The method of embodiment 84, wherein a compound is selected for further testing if:

(a) the level of MAP2 in the neuronal culture is increased by ≥30%;
(b) the level of Synapsin 1 or Synapsin 2 is increased by ≥30%;
(c) the level of CUX2 is increased by ≥30%; and/or
(d) the level of beta III tubulin is increased by ≥30%; when compared to a corresponding neuronal culture not contacted with the compound.

Embodiment 87. The method of embodiment 84 or 86, wherein a compound is determined to be neuroprotective if

(a) the level of MAP2 in the neuronal culture is increased by ≥30%;
(b) the level of Synapsin 1 or Synapsin 2 is increased by ≥30%;
(c) the level of CUX2 is increased by ≥30%; and/or
(d) the level of beta III tubulin is increased by ≥30%; when compared to a corresponding neuronal culture not contacted with the compound.

### Examples

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1. Generation of a High-Throughput, Automated iPSC-Derived Human Neuron Culturing Platform

This example shows the workflow and exemplary applications of a high-throughput, automated iPSC-derived human neuron culturing platform.

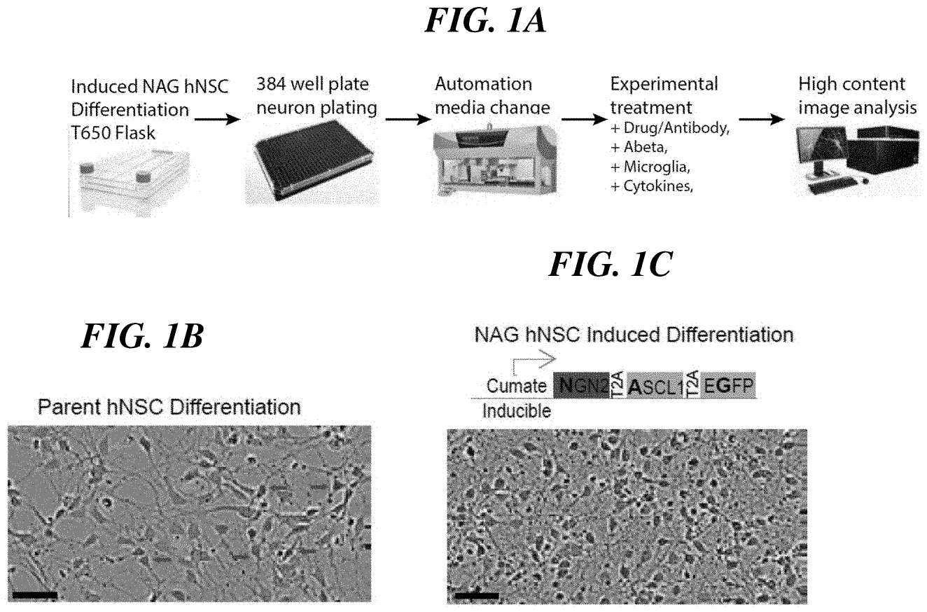

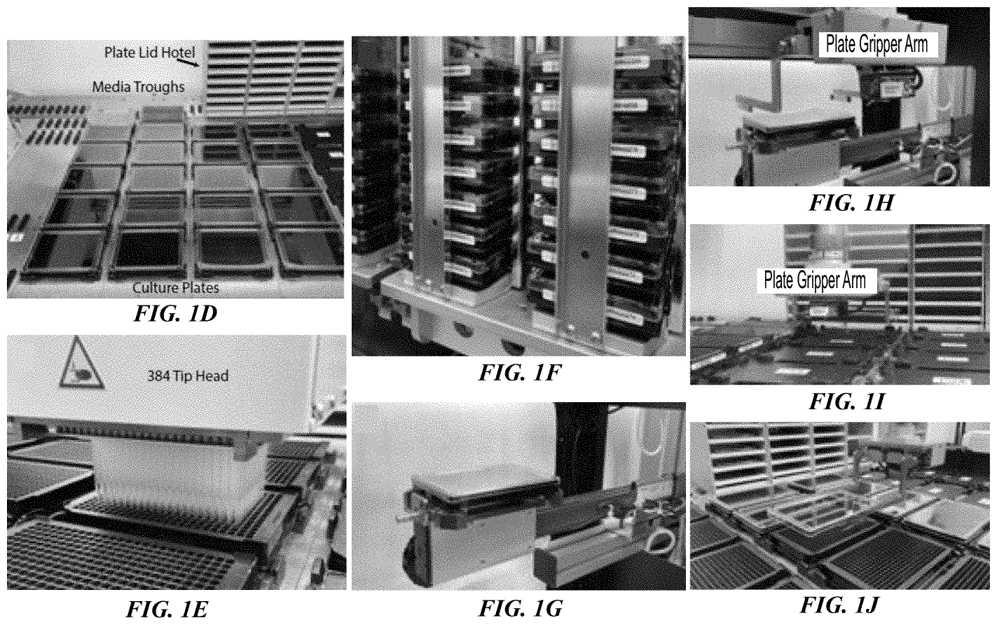

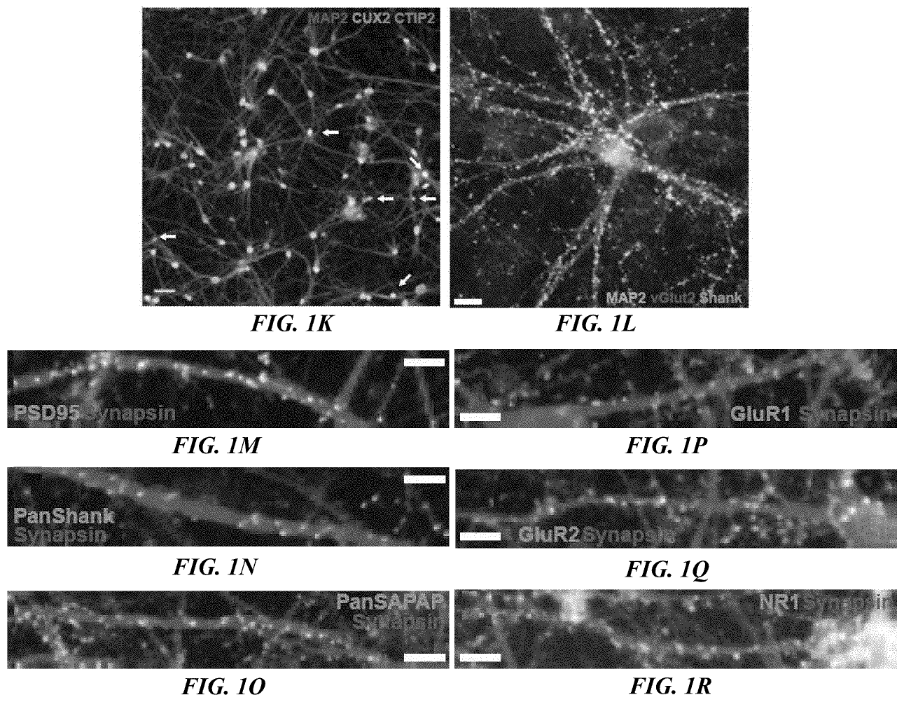

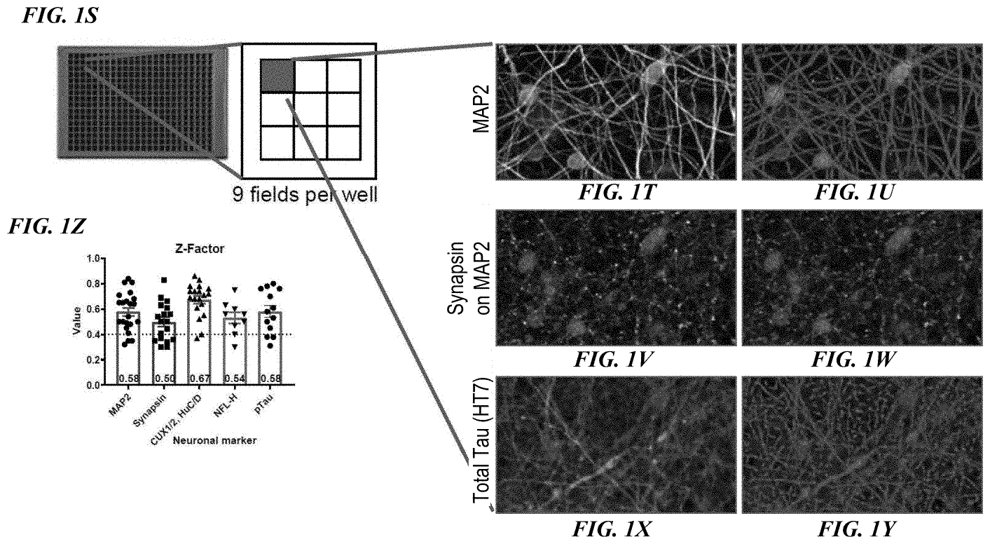

FIG. 1A shows the workflow of a high-throughput, automated iPSC-derived human neuron culturing platform, as applied to the methods described herein. The workflow (FIG. 1A) started with induced iPSC neuron differentiation in large batches (100-200 million cells), which were then replated into 384 well imaging plates. Fluent® Automation workstation (Tecan) was used for multiple liquid-handling steps such as cell plating, media changes, experimental treatment, and cell fixation to achieve systematic, reproducible, and precise neuron handling. The multiplex-stained cells were then scanned and quantified using an automated high content imaging system (IN Cell Analyzer 6000; GE Healthcare).

To achieve accelerated, synchronized, and homogeneous differentiation, two different human iPSC-derived neural stem cells (NSC) lines expressing NGN2, ASCL1, and a green fluorescent protein (GFP) reporter under a cumate-inducible system, were generated. To generate NSC cell lines, multiple human induced pluripotent stem cell-derived neural stem cell lines (iPSC-NSCs) were obtained and tested for basal NSC maintenance and intrinsic neuronal differentiation quality in small scale (Axol, Millipore, Thermofisher, MTI global, Tempo Bioscience, Roche). iPSC-NSC from MTI Global Stem (HIP™ Neural Stem Cells, BC1 line) and Roche (gift from Christoph Patsch, Roche (Basel, Switzerland)) were chosen based the following criteria: 1) able to be maintained a homogenous NSC morphology beyond 40 passages; 2) >80% neuronal differentiation efficiency; 3) fast growth rate, at least 1:3 expansion/split ratio; and, 4) no remaining progenitor cells after differentiation. NSCs were transfected to stably and express inducible ASCL1 and NGN2, transcription factors whose expression has been shown to increase differentiation efficiency in combination with differentiation media. Transcription factors ASCL1, NGN2, and EGFP were cloned into a vector containing a cumate inducible promoter (Systembio), then stably transfected using Neon electroporation. Both cell lines were cultured according to manufacturer's instructions. Briefly, cells were cultured on flasks coated with a 1:100 Geltrex (Thermofisher) solution for at least 1 hour in a 37° C. cell incubator. Cells were grown in Neural Stem Cell Growth Media (0.5×DMEM/F12, 0.5× Neurobasal™ (ThermoFisher), 1×B27 no Vitamin, 1×N-2, 20 ng/mL BDNF, 20 ng/mL FGF-basic, 20 ng/mL EGF, 0.5 mM GlutaMAX™ (Gibco), 0.11 mM 0-mercaptoethanol, 1× normocin, 50 U/mL penicillin-streptomycin) and 0.75 μg/mL Puromycin selection marker, at 37° C. 5% CO₂ cell culture incubator. Cells were passaged every 3-4 days when confluent using TrypLE™ Express Enzyme (Gibco) and split at no more than a 1:3 ratio depending on cell density.

The generated NSC cell lines were then differentiated. Briefly, NAG-NSCs were grown until confluent and then detached using TrypLE™ Express Enzyme (Gibco) and plated onto a T-650 flask coated with 50 μg/mL poly-D-lysine and 10 μg/mL mouse laminin. Cells were plated at a density of 0.7×10⁸-1.0×10⁸ cells/flasks in Neuron Differentiation Media (0.5×DMEM/F12, 0.5×Neurobasal™ (Thermofisher), 1× B27 with Vitamin A, 1×N2, 5 μg/mL cholesterol, 1 mM creatine, 100 μM ascorbic acid, 0.5 mM cAMP, 20 ng/mL BDNF, 20 ng/mL GDNF, 1 μg/mL laminin, 0.5 mM GlutaMAX™ (Thermofisher), 1× normocin, 50 U/mL penicillin-streptomycin) supplemented 100 μg/mL cumate, 1 μM PD0332991 cell cycle inhibitor, and 10 μM Y27632 Rock inhibitor. Cells were differentiated for 5-7 days, with one half volume differentiation media changed every 3 days. Once differentiated, cells were detached using AccuMAX™ (Innovative Cell Technologies) supplemented with 5% trehalose dihydrate, 1 U papain, 10 μM Y27632, and 8 mM kynurenic acid. Cells were plated using Tecan Fluent® Automation workstation into 384 well or 96-well CellCarrier Ultra imaging plates (PerkinElmer) coated with 50 μg/mL poly-D-lysine and 20 μg/mL recombinant human laminin in Neuron Differentiation Media supplemented with 10 μM Y276342 Rock Inhibitor, and 1× RevitaCell™ (Gibco).

Primary human astrocytes were cultured and passaged according to manufacturer's instructions in Primary Human Astrocyte Medium (1×DMEM/F12, 1×N-2, 10% FBS, 1× normocin, 50 U/mL penicillin-streptomycin) on T-650 flasks coated with 1:100 Geltrex™ (Thermofisher) solution for at least 1 hour in a 37° C. cell incubator. Full volume of media was changed every 3-4 days until cells were confluent. Cells were passaged when confluent using TrypLE™ Express Enzyme (Thermofisher) and split at a ratio of up to 1:6.

Astrocytes were then validated. Briefly, primary human astrocytes were detached using Accumax (Innovative Cell Technologies) and seeded onto 384-well plates coated with 1:100 Geltrex™ (Thermofisher) solution for at least 1 hour in a 37° C. cell incubator. Cells were seeded at a density of 2,000 cells/well in Neuron Maintenance Medium (1× BrainPhys™ Basal (StemCell Technology), 1× B27 with Vitamin A, 1×N-2, 5 μg/mL cholesterol, 1 mM creatine, 10 nM β-estradiol, 200 nM ascorbic acid, 1 mM cAMP (Sigma-Aldrich), 20 ng/mL BDNF, 20 ng/mL GDNF, 1 μg/mL Laminin, 0.5 mM GlutaMAX™ (Thermofisher), 1 ng/mL TGF-β1, 1× normocin, 50 U/mL penicillin-streptomycin). Cells were placed in 37° C. cell incubator for 24 hours to allow for attachment. Aβ42 and antibody treatments were added as described in other Examples. Astrogliosis was validated was validated by immunostaining of the following markers: Guinea Pig anti GFAP (1:500), Rabbit anti EAAT1 (1:500), Rabbit anti vimentin (1:500), Rabbit ALDH1L1 (1:500).

Cumate induction in combination with cell cycle inhibition (PD0332991) in both NSC lines generated homogeneous iPSC neurons within 7 days, as expected (FIGS. 1B-1C). After neuron differentiation and replating, primary human astrocytes (Thermofisher) were added to the culture to promote neuronal health and maturation, 5-10 days after neuron replating. Astrocytes were detached using AccuMAX™ (Innovative Cell Technologies) and plated using Tecan Fluent© Automation workstation into 384-well or 96-well plates containing differentiated and replated neurons at a density of 4,000 or 20,000 cells/well, respectively, Neurons and astrocytes were co-cultured in 384- or 96-well Cell Carrier Ultra plates (PerkinElmer) in Neuron Maintenance Medium and half of the volume of culture media was changed using Tecan Fluent® Automation liquid handling workstation every 3-4 days for at least 8 weeks and up to 6 months before subsequent experimentation. Tecan Fluent© Automation workstation was programmed to utilize its features of automated tip loading, lid removal, and to aspirate half volume of culture media and add new culture media for up to 30 plates at a time. Barcode-operated plate storage incubator technology was integrated into the system for plate organization and retrieval.

The Fluent© Automation workstation was used to maintain long-term iPSC neuronal cultures in 384-well plates. Convenience features of the automated workstation allowed walk-away implementation, to maintain consistent and healthy neurons up to 6 months (FIGS. 1D-1J).

Neurons from both NSC lines were evaluated by immunofluorescence staining. Briefly, cells were fixed with 4% PFA and 4% sucrose at room temperature for 20 minutes using Bravo automation. Fixed cells were then washed 2 times with PBS using Biotek 406 microplate washer (Beckman Coulter), followed by permeabilization and blocking by incubation with a solution containing 1×PBS, 0.1% Triton X-100, 2% donkey serum, and 1% BSA at room temperature for 30 minutes. Blocking solution was removed and cells were incubated with primary antibodies in blocking solution overnight at 4° C. After washing 6 times with PBS on Biotek 406 cell plate washer (Beckman Coulter), cells were then incubated with fluorophore-conjugated secondary antibodies for 1 hour at room temperature in the dark to avoid photobleaching. Cells were then washed 6 more times in with PBS before imaging. Fluorescent images were captured using IN Cell 6000 confocal microscope (GE Healthcare Life Sciences). The image analysis was performed with IN Cell 6000 analysis software.

Resulting neurons from both NSC lines were homogenous, upper-layer cortical neurons, with over 95% expressing CUX2, and with only 2-5% expressing CTIP2 or SATB2 (FIG. 1K). Neurons also had extensive synaptic connections and expressed several pre- and post-synaptic markers: PSD95, SHANK, PanSHANK, GluR1, GluR2, vGLUT2, Synapsin 1/2, PanSAPAP, and NR1 (FIGS. 1L-1R). The use of 384-well plates enabled simultaneous testing of numerous experimental conditions, each with four biological replicates (n=4). An IN Cell Analyzer 6000 and ImageXpress Micro Confocal were used for automated confocal image acquisition. Nine fields were imaged per well, which covered 70% of the area, and captured over 1,000 neurons (FIG. 1S). Image analysis scripts provided precise segmentation of markers of interest including dendrites (MAP2), cell bodies (CUX2), axons (Tau, p-Tau), and synapses (Synapsin 1/2) (FIGS. 1T-1Y). To characterize the variability of the assay performance, average Z-factors (a measure of assay reliability) were calculated from multiple batches and experiments (about 10-20 total) for the aforementioned assays. As shown in FIG. 1Z, the average Z-factors ranged from 0.5-0.7.

### Example 2. An In Vitro Human Neuron Model of Alzheimer's Disease (AD) Recapitulates AD Pathology Hallmarks and Kinetics

This example shows that Alzheimer's Disease (AD) can be studied in a controlled, in vitro system of human neurons. In particular, this example shows that an in vitro system of human neurons can effectively recapitulate AD pathology hallmarks and kinetics.

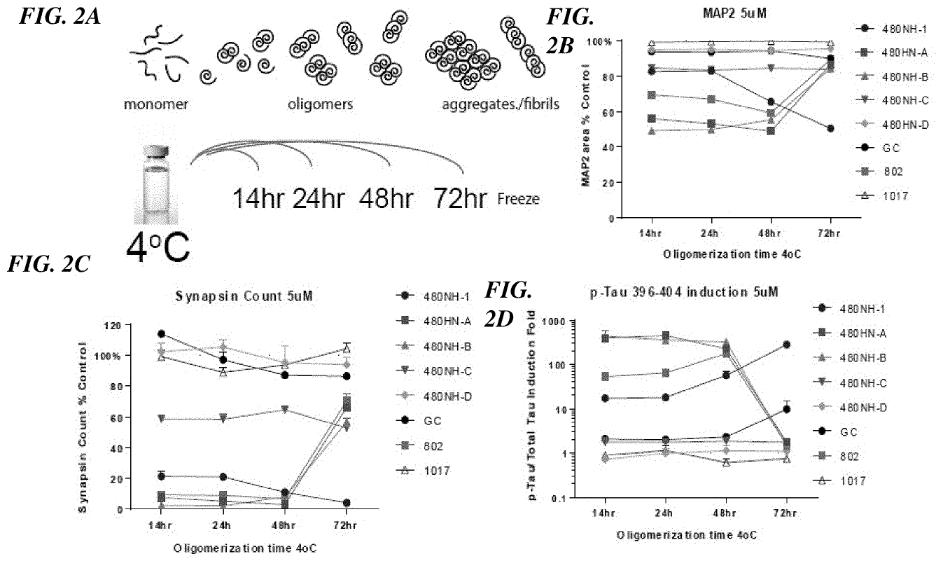

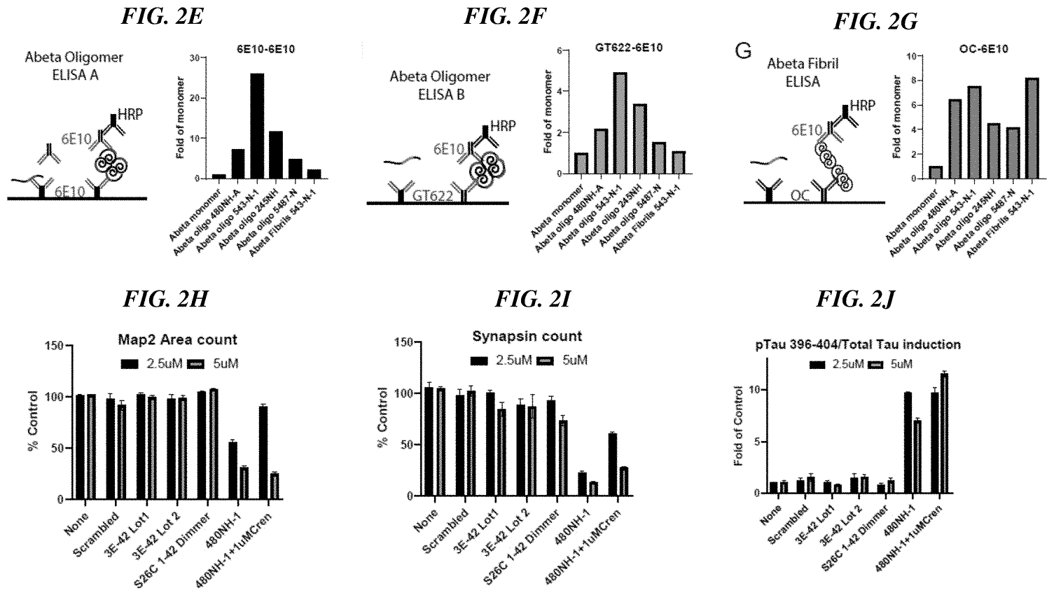

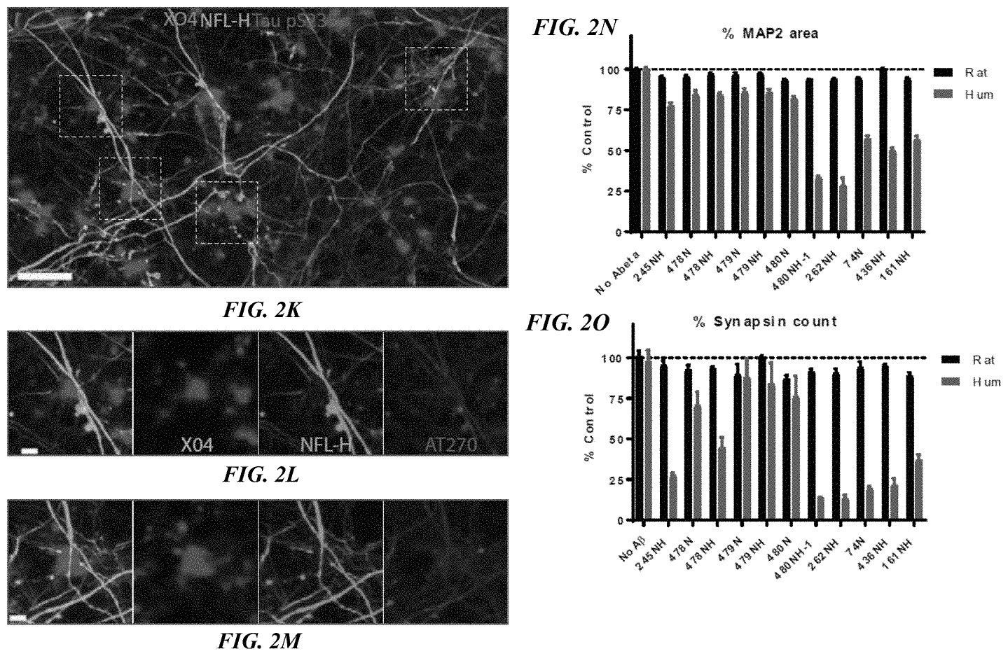

To investigate whether AD pathologies can be studied in a controlled, in vitro system of human neurons, cultured human iPSC-derived neurons were treated with synthetic Aβ42 oligomers, prepared by oligomerization of Aβ42 monomers at 4° C. following previously published protocols (FIG. 2A; following Stine et al., 2011). Briefly, AggreSure™ β-Amyloid (1-42), human monomers (Anaspec) were resuspended in DMSO followed by PBS to form a 100 μM solution. Aβ42 monomers were subsequently incubated at 4° C. for 24 hours, then frozen at −80° C. to stop the oligomerization process. Five to six lots of Aβ42 monomers were screened at time and assessed for neurotoxic and the degree of toxicity. About twenty lots for screened in the course of 4 years. For fluorescent Aβ42 oligomer experiments, Beta-Amyloid (1-42), HiLyte™ Fluor 555-labeled, Human (Anaspec) was used. For pHrodo experiments, Beta-Amyloid (1-42) buman was labeled with pHrodo™ Green AM Intracellular pH Indicator (Invitrogen) according to manufacturer's protocol.

To reduce variability between Aβ42 oligomer preparations, the oligomerization duration was optimized, and Aβ42 monomer lots that demonstrated consistent AD pathologies in neurons upon treatment were selected, displaying: synapse loss, pTau induction, and neuronal loss (FIGS. 2A-2J). Aβ oligomer selective and Aβ fibril selective ELISAs were additionally developed to confirm the generation of oligomer species (FIGS. 2E-2G). Briefly, to detect for the presence of oligomeric Aβ42, a 6E10-6E10 assay utilizing the same anti-AP42 (6E10) as both capture and detection, to selectively bind to oligomeric species containing more than one exposed 6E10 binding site, was used. To further test for oligomeric species, a GT622-6E10 assay using AD-oligomer specific antibody (GT622) as capture, and pan-Aβ antibody (6E10) as detection was used. Finally, the presence of fibril species was tested using Aβ fibril selective antibody clone (OC) as capture and pan Aβ antibody (6E10) as detection.

Aβs to be tested were prepared as previously described in Example 1. Clear, flat bottom immuno nonsterile maxisorp 384-well plates were coated with 100 ng/mL of different anti-AP42 antibodies (6E10; GTX622; OC), in 0.05 M sodium carbonate buffer, pH 9.6, and allowed to set overnight. Plates were washed 3× with 0.05% Tween-20 in 1×PBS, then blocked in 0.5% BSA+15 PPM proclin in 1×PBS, pH 7.4 for 1 hour. All samples were quantified against Aβ42 monomer diluted to 1 μg/mL in 0.5% BSA+0.05% Tween-20+0.35M NaCl+0.25% CHAPS+5 mM EDTA in 1×PBS, pH 7.4 (Assay Buffer), then diluted two-fold to 15.625 ng/mL. Sample Aβ42 oligomers were diluted to 1 μg/mL in Assay Buffer, then diluted three-fold to 37 nM. The blocked plate was then washed 3× in 0.05% Tween-20 in 1×PBS, then samples, standards, and controls were added and incubated at 4° C. overnight. After sample incubation, the plate was washed 6× with 0.05% Tween-20 in 1×PBS, then 100 ng/mL conjugate antibody in Assay Buffer (6E10) was added and incubated for 1 hour at room temperature. After incubation, the plate was washed 6× with 0.05% Tween-20 in 1×PBS, then streptavidin-poly 80 HRP detection antibody was added at a dilution of 1:10,000 in Assay Buffer and incubated for 45 minutes at room temperature. After incubation, the plate was washed 6× with 0.05% Tween-20 in 1×PBS and TMB substrate was added to each well, then incubated for 10-15 minutes. After appropriate color developed, 1M H3P04 was added to quench the reaction, Finally, the plate optical density (O.D.) was read at 450-630 nm.

The preps were determined to contain a heterogeneous mixture of both soluble oligomer and fibrils, and thus were referred to as “soluble Aβ42 species” (Aps). Aβs-induced neurotoxicity is specific to human neurons, as primary rat cortical neurons treated with several different lots of Aβ42 oligomer preparations did not show reduction in dendrites or synapses (FIGS. 2N-2O).

Prior to experimentation, media volume in wells containing neurons was equalized with liquid handling automation (Bravo) in order to ensure precise control of concentrations. All Aβ42 oligomers, anti-Aβ, small molecules, inflammatory cytokines were prepared at 10× concentration and added to Neuronal Culture Media at an appropriate volume. For the repeated dosing experiments, the media were refreshed 50% at each dosing first, before adding Aβ42 oligomers, and/or anti-Aβ antibody at the specified final concentration.

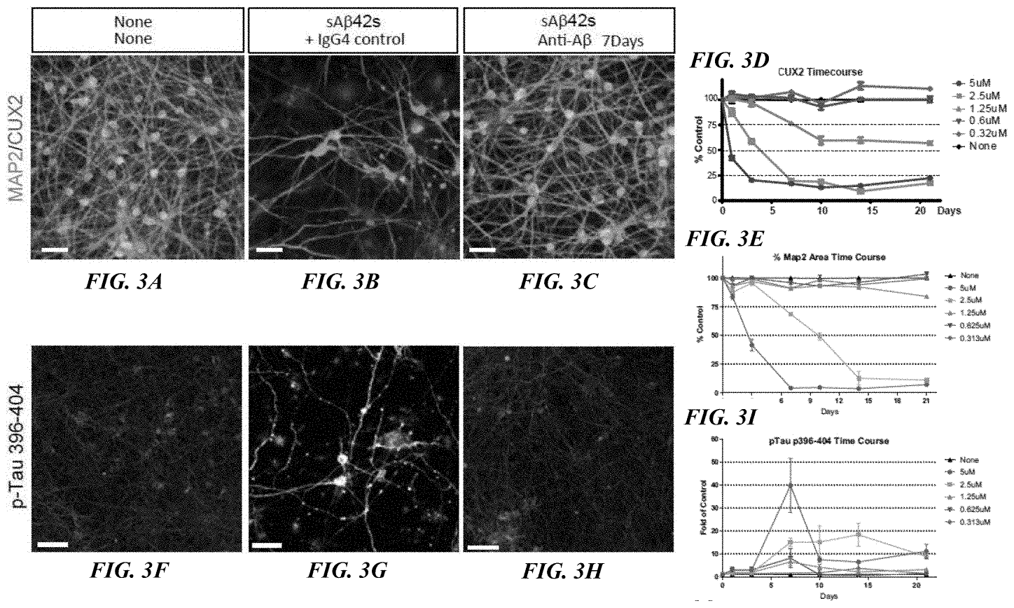

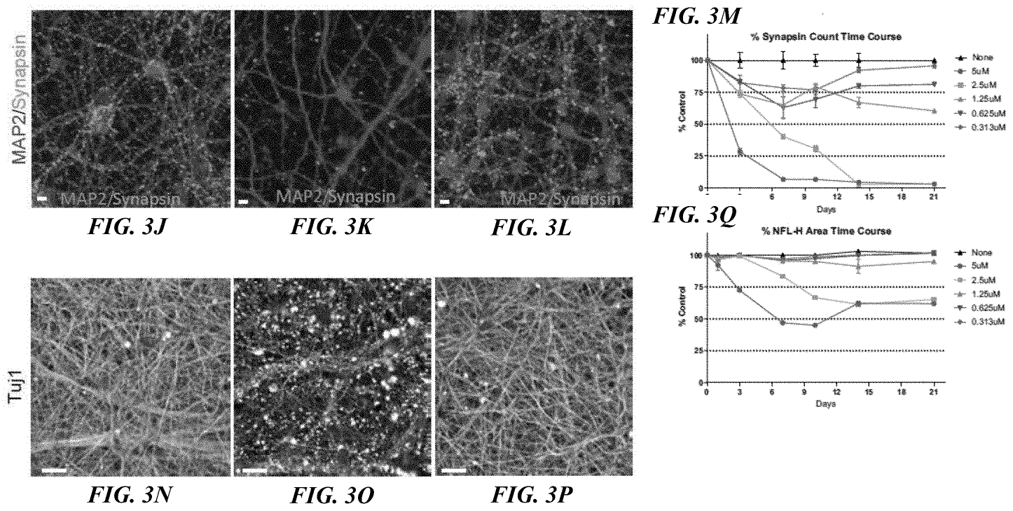

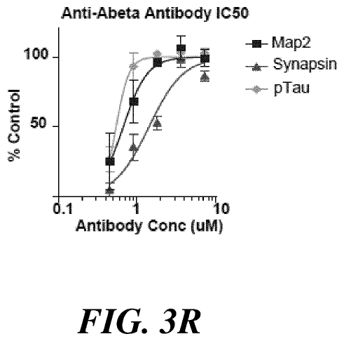

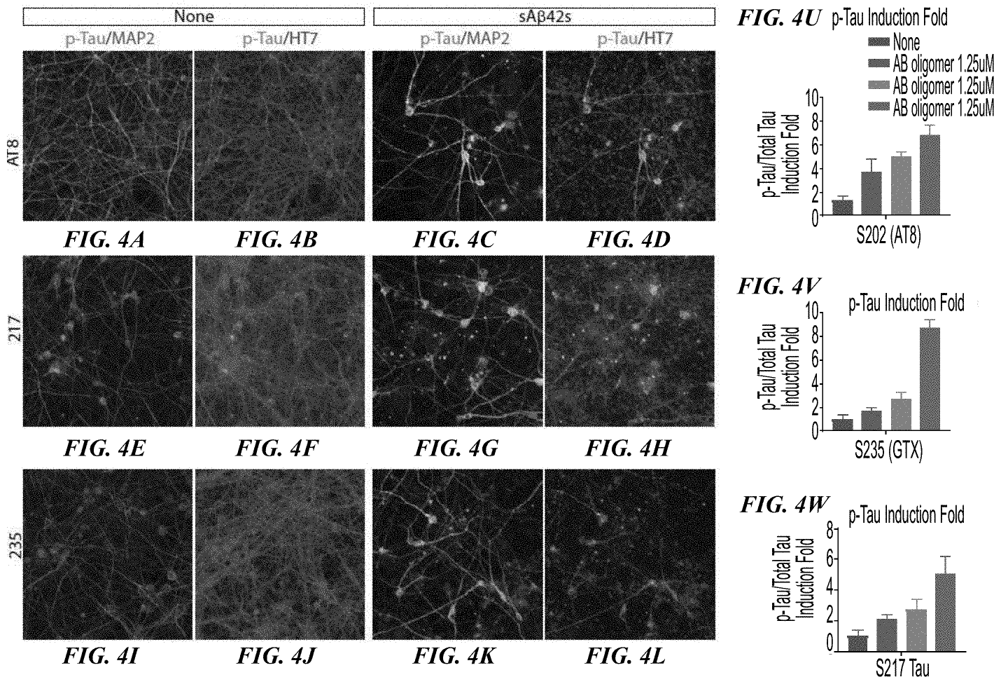

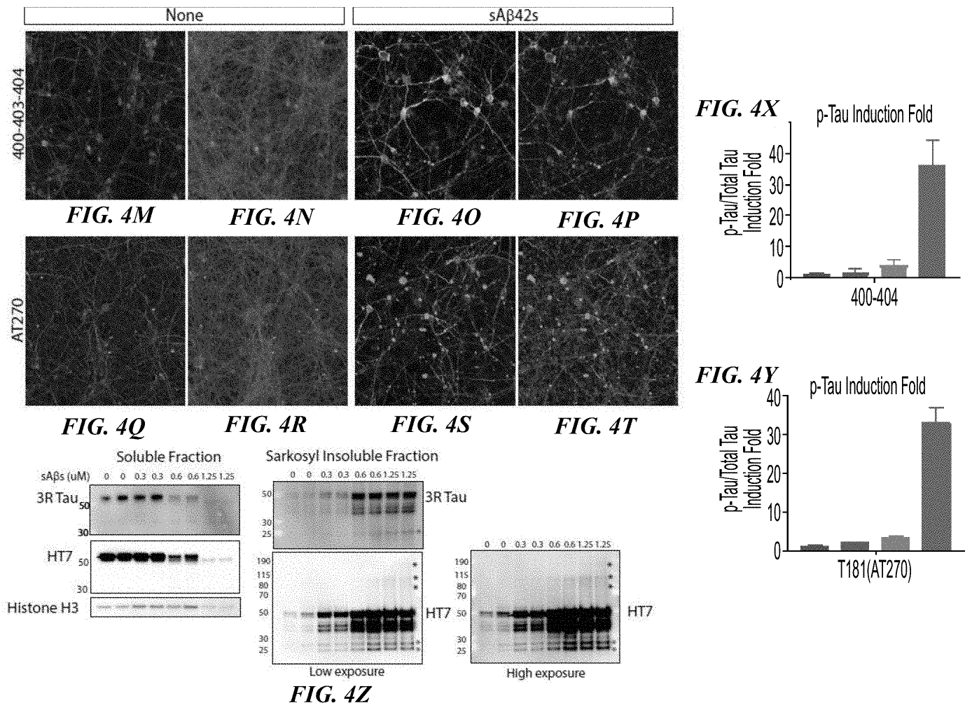

FIGS. 3A-3Y and FIGS. 4A-4W show that neurons incubated with 5 μM Aβs show marked loss of synapses, dendrite reduction, axon fragmentation, induction of tau hyperphosphorylation (S396/404), and dramatic cell death at 7 days. Several additional tau phosphorylation sites observed in AD, S396/404, S217, S235, S400/T403/S404, and T181, were hyperphosphorylated when treated with Aβ42 oligomers (FIGS. 4V-4Z). Additionally, repeated treatment of 300 nM Aβs soluble species for 3 weeks increased total tau (HT7) in the Sarkosyl insoluble fraction that are 3R-repeat positive (FIG. 4Z). The iPSC neurons were negative for 4R-repeat tau at this age (data not shown). Interestingly, both tau fragmentation and faint higher molecular tau bands in the sarkosyl-insoluble fraction were observed, suggesting the formation of detergent-insoluble higher molecular weight tau aggregates. Neurotoxicity was blocked by co-treatment with anti-Aβ antibody in a dose-dependent manner, indicating that pathological hallmarks of AD observed in the in vitro human neuron model are A-specific (FIGS. 3C, 3H, 3L, 3P, and 3R). Using this quantitative platform, the half-maximal inhibitory concentration (IC50) for anti-Aβ antibody rescue of MAP2, synapsin, and pTau induction was generated (FIG. 3R). The results indicate that synapse rescue is linear, whereas the MAP2 and p-Tau induction rescues are more indicative of a thresholded response with sharp transitions. Furthermore, the IC50 of synapse rescue is ˜1.4 μM at 5 μM soluble Aβ42 species, suggesting a stoichiometric relationship between anti-Aβ antibodies and Aps, resulting in a required 1:2 molar ratio for complete blocking.

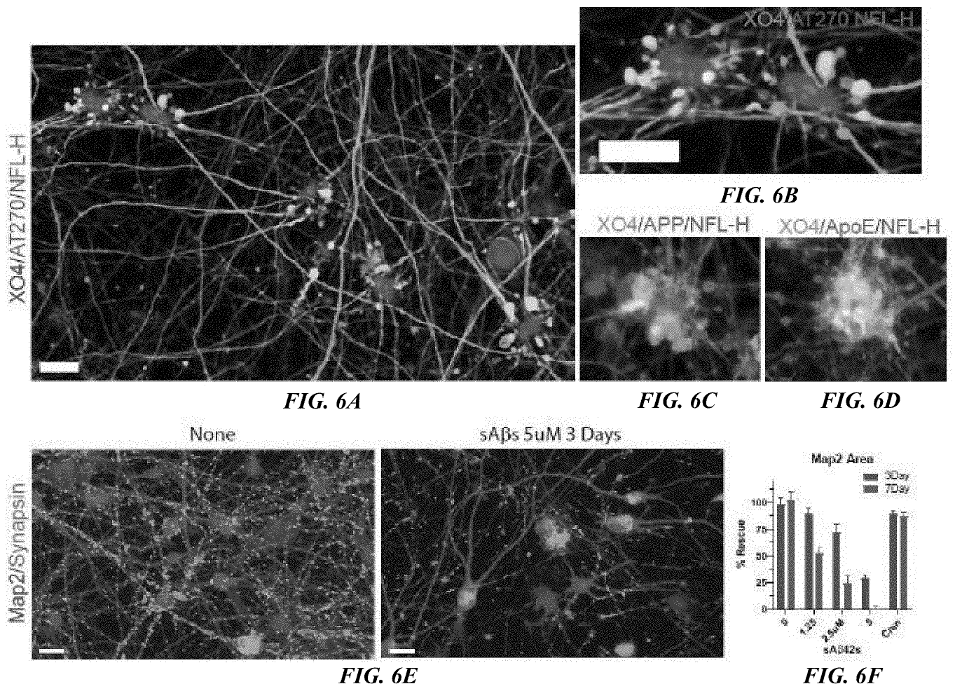

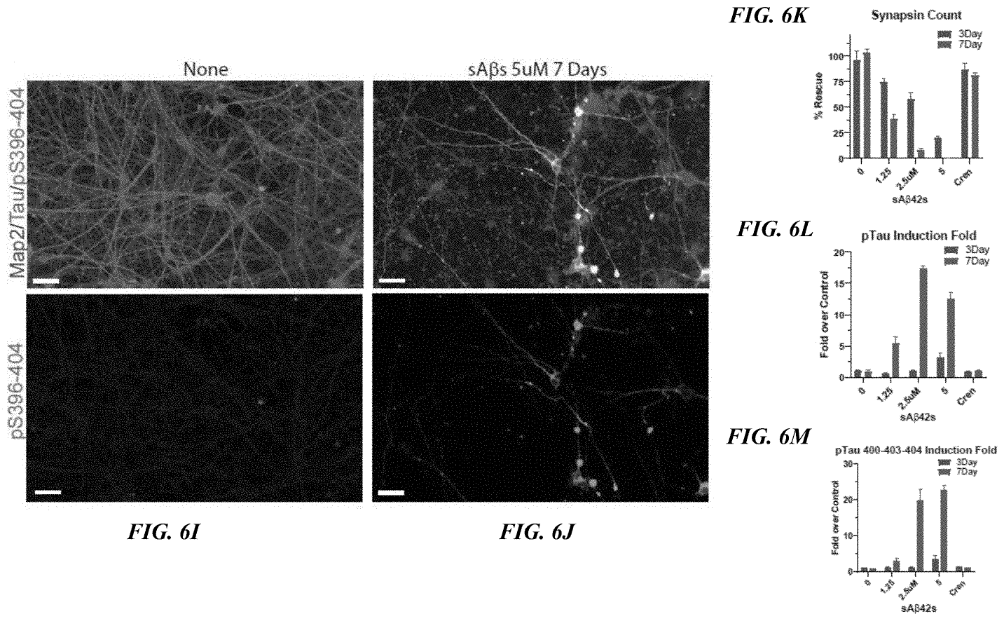

To characterize the kinetics and the effects of soluble Aβ42 species on neurotoxicity, a 21-day time course experiment with single doses of increasing Aβs concentrations was performed. Phenotypes associated with Aps-neurotoxicity were dose-dependent and progressive; higher doses resulted in faster pathology development and neuronal loss (FIGS. 3D, 3E, 3I, 3M, and 3Q). The most sensitive and earliest phenotype to appear is synapse loss; a 25% reduction in synapses at 0.3 μM Aps, whilst other neurodegenerative markers are unaffected (FIGS. 3D, 3E, 3I, and 3Q). At this lowest synapse damage, the neurons could recover after 21 days. Interestingly, the neurotoxic response of dendrite and axon reduction to the Aβs has a threshold effect, whereby at 1.25 μM there is no effect on dendrite or axon reduction even though there is a sustained loss of synapses and CUX2 nuclear expression (FIGS. 3D, 3E, 3M, and 3Q, green line). Induction of pTau induction seemed to be more proximal to neuronal death, as we were unable to capture the initial induction of pTau when neurons died rapidly at high sAβ42s concentrations. These findings were also recapitulated in a second iPSC derived neuron lines (FIGS. 6A-6M), indicating the robustness of the phenotypes.

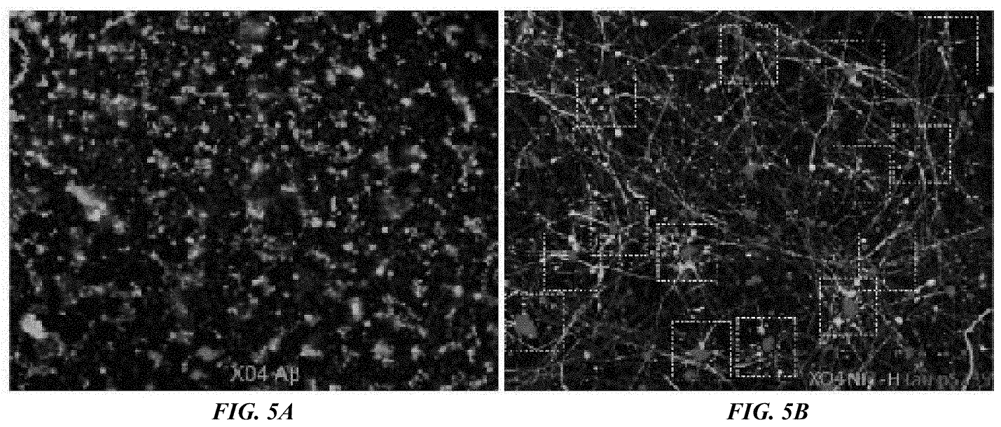

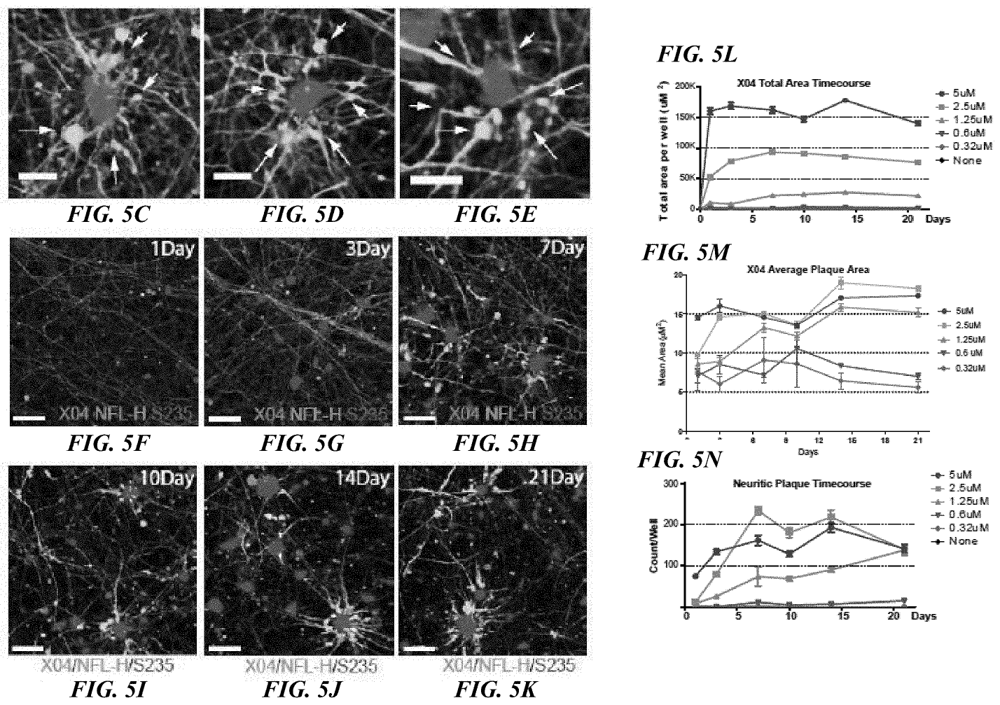

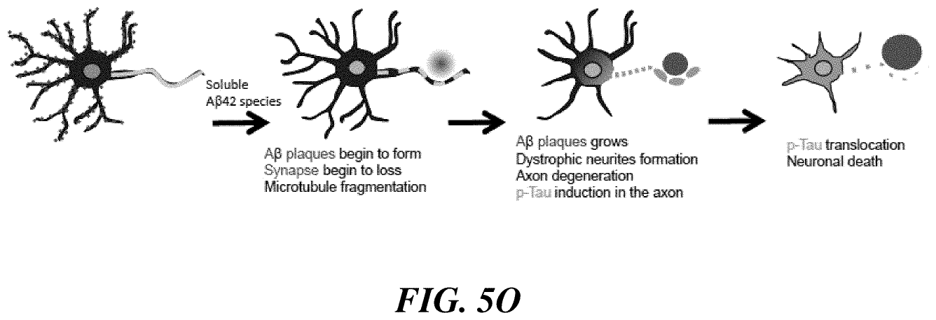

These data show that the model not only exhibits hallmarks of human AD pathologies in response to soluble Aβ42 species, but also revealed a sequence of degeneration events, beginning with synapse loss, axon fragmentation, and dendritic atrophy, followed by p-Tau induction resulting in severe neuronal loss (FIG. 5O).

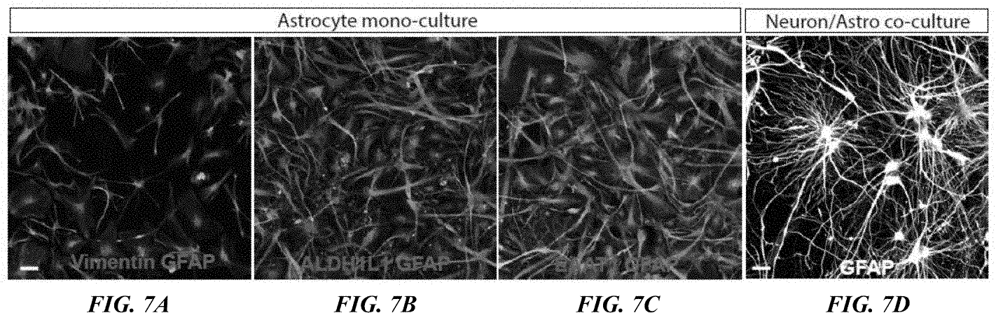

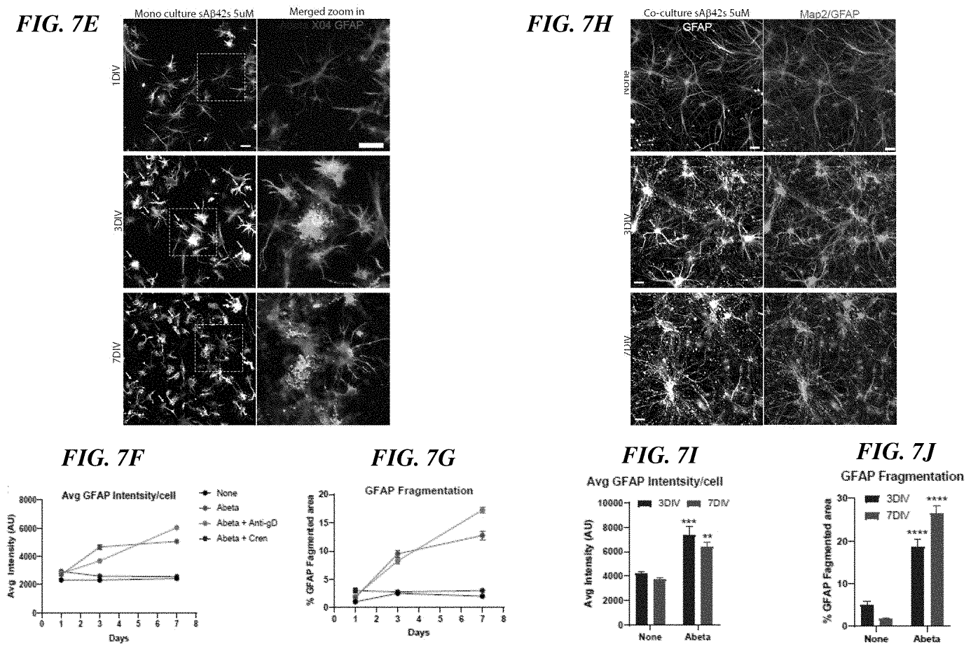

In response to CNS damage and neurodegeneration, astrogliosis can often be observed, which is commonly characterized by pronounced structural changes in astroglia that result in upregulation of glial fibrillary acidic protein (GFAP), and have been shown as a potential serum biomarker for AD. Human astrocyte cultures in the in vitro human neuron model of AD were similarly shown to express multiple astrocyte markers such as GFAP, vimentin, ALDH1L1, and EEAT1 in the characteristic astrocyte morphology (FIGS. 7A-7C). After extended culturing with human iPSC neurons, increasingly elaborated astrocyte processes are observed (FIG. 7D). In response to sAβ42s, human astrocytes show a 2-3 fold elevation in GFAP expression both in mono-culture and in co-culture with neurons (FIG. 7E-7J). Increased GFAP fragmentation was additionally observed (FIGS. 7G and 7J), which has been shown to be cleaved by caspases during CNS injury.

### Example 3. iPSC-Derived Neurons and Astrocytes Recapitulate Aβ Plaque Formation

This example shows that iPSC-derived neurons and astrocytes recapitulate Aβ plaque formation.

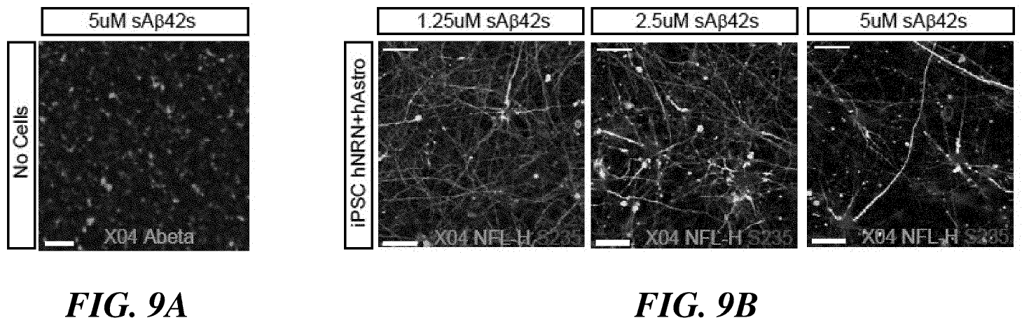

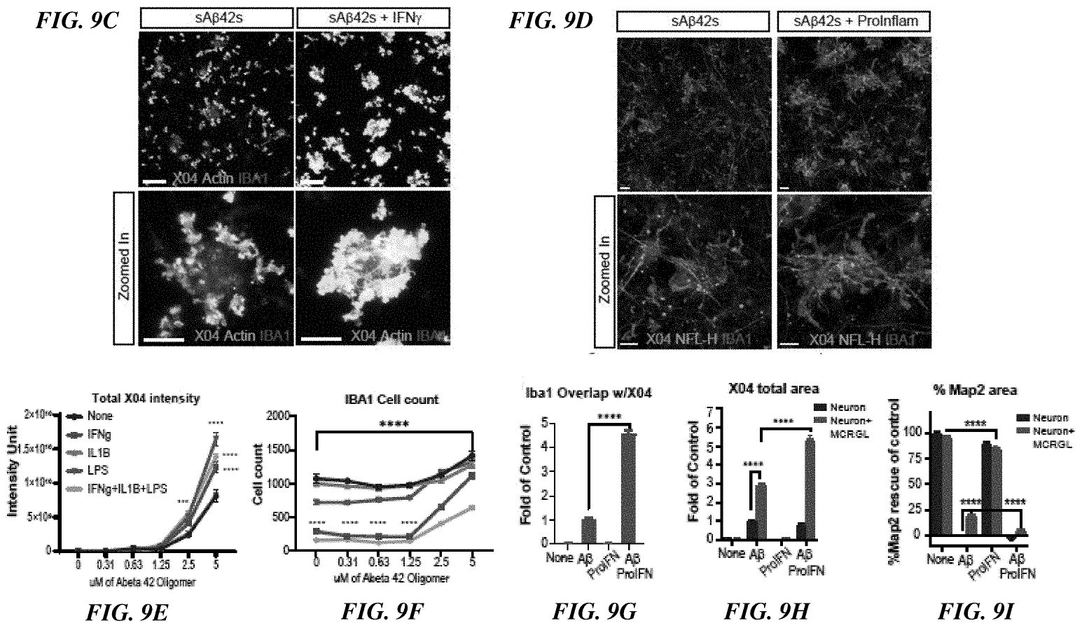

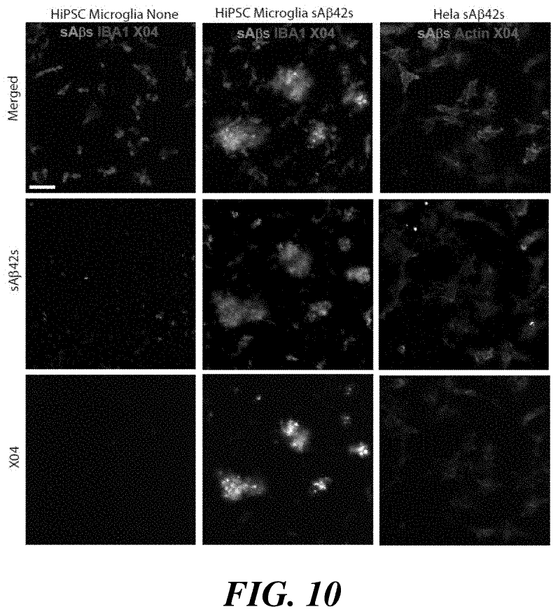

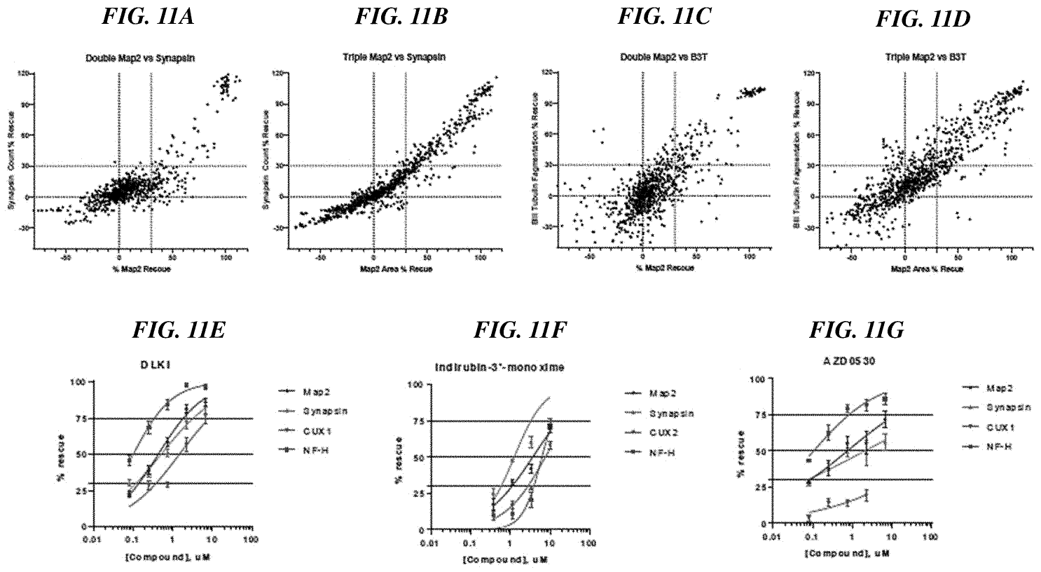

After observing hallmark AD pathologies in the in vitro human neuron model upon treatment with Aβ oligomers, the model was next evaluated for the ability to recapitulate Aβ plaque formation. In the presence of iPSC-derived neurons and primary astrocytes, A3-aggregated structures were positive for Methoxy-X04, a commonly used Aβ plaque-binding small molecule dye (FIG. 5A). To confirm that the plaque-like structures were made by cells, the empty culture wells treated with Aβ oligomers were also stained. Smaller, morphologically distinct aggregates of Aβ that were X04-dye negative were observed in the empty culture wells (FIG. 9A). These distinct aggregates are likely the result of Aβ oligomers continuing to oligomerize and fall out of solution onto the culture plates. In contrast, incubation with Hela cells with Aβs do not form the same AD-aggregated structures positive for Methoxy-X04 as we observed in human iPSC neurons (FIG. 10).

Further characterization showed that a subset of X04-positive Aβ plaque-like structures were surrounded by dystrophic neurites marked by neurofilament heavy chain (NFL-H) axonal swelling and phosphorylated Tau (S235) positive blebbings (FIGS. 5B-5E). These structures are very similar to Aβ plaques with neuritic dystrophy observed in human AD postmortem brain sections. Importantly, neuritic plaque-like structures were also observed in the second iPSC NSC line derived neurons (FIGS. 6A-6M), indicating the robustness of this phenotypes.

The in vitro AD neuritic plaque like structures were also positive for ApoE and APP (FIGS. 6C-6D). To further characterize this finding, a time course experiment with increasing concentrations of Aβs was performed. Time course analysis showed that individual Aβ plaques grew in size and then peaked over 7 days (FIGS. 5F-5L). The growth of Aβ plaques was accompanied by emergence of dystrophic neurite marker morphology 3 days after plaque formation which worsened over time, indicating that neurons might form dystrophic neurites as a reaction to direct Aβ plaque exposure (FIGS. 5F-5N). Interestingly, astrocyte monocultures were also reactive to soluble Aβ species, and formed large X04 positive Aβ structures. These structures were large and fibrous (FIG. 7E), and were not of the characteristic compacted neuritic plaque morphology.

Taken together, the data suggests that Aβ42 soluble species, in the presence of neurons and astrocytes, leads to X04-positive neuritic plaque formation, ultimately resulting in neuritic dystrophy.

### Example 4. Human iPSC-Derived Microglia Lose Neuroprotection in a Neuroinflammatory Context

This example shows that the microglia of human iPSC-derived neurons lose neuroprotection in a neuroinflammatory environment, such as the environment surrounding AD plaques observed in human AD.

Since Aβ plaques observed in human AD are often surrounded by microglial cells in a neuroinflammatory environment, it was investigated whether iPSC-derived human microglia alone could generate and surround Aβ plaques, and whether neuroinflammatory cytokines could modulate microglial behavior.

iPSC derived microglia were obtained and screened for microglia marker expression. iPSC microglia were then differentiated. Briefly, iPSCs were treated with BMP, FGF and activin for 2-4 days to induce mesoderm fate, then treated with VEGF and supportive hematopoietic cytokines for 6-10 days to generate hematopoietic progenitors (HPCs). HPCs were seeded onto matrigel-coated flasks, and treated with IL-34, IDE1 (TGF-β1 agonist), and M-CSF for 3-4 weeks to differentiate into microglia. Human iPSC microglia were validated by immunostaining of the following markers: Goat anti-TREM2 (1:500), Mouse anti-MERTK (1:500), Rabbit anti-IBA1 (1:1000), Rabbit anti-TMEM119 (1:500), CD33 (1:500), CX3CR1 (1:500), CD64 (1:500), P2RY12 (1:500), CD32 (1:500), PU.1 (1:500).

Frozen cells were thawed and immediately seeded at a density of 8,000 cells/well of a 384-well plate onto 8-week old neuron-astrocyte co-culture in Microglia media (BrainPhys™ neuronal media (Stem Cell Technologies) supplemented with 1× B27 with vitamin A, 1×N2 Plus Media Supplement (R&D Systems), 20 ng/mL BDNF, 20 ng/mL GDNF, 1 mM creatine, 200 nM L-ascorbic acid, 1 μg/mL mouse laminin, 0.5 mM GlutaMAX™ (Thermofisher), 0.5× penicillin-streptomycin, 1× normocin, 5 ng/mL TGF-β, 100 ng/mL human IL-34, 1.5 μg/mL cholesterol, 1 ng/mL gondoic acid, 100 ng/mL oleic acid, 460 μM thioglycerol, 1× insulin-transferrin-selenium, 25 ng/mL rhM-CSF, 5.4 μg/mL human insulin solution).

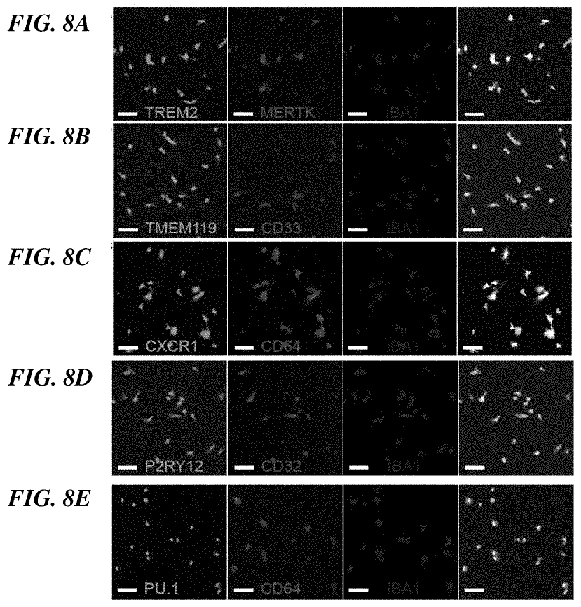

iPSC-derived microglia used in this study expressed known markers and exhibited typical ramified morphology (FIGS. 8A-8E), and were also capable of generating and surrounding X04-positive Aβ plaques in vitro in a dose-dependent manner (FIGS. 9C and 9E). iPSC-derived microglia stimulated with pro-inflammatory cytokines interferon-gamma (IFNγ), interleukin 1β (IL-1β), and lipopolysaccharide (LPS), demonstrated increased plaque formation as measured by total X04-positive area and intensity, and additionally surrounded Aβ plaques more closely (FIGS. 9C and 9E). Furthermore, microglial cell number was increased, as measured via ionized calcium-binding adapter molecule 1 (IBA1)-positive cell count, suggesting a microgliosis response (FIG. 9F).

After confirming that iPSC microglia demonstrated behavior similar to in vivo observations, microglia was co-cultured with neuron-astrocyte AD model conditions, and inflammatory cytokines were added, to understand cell-cell dynamics in an inflammatory, Aβ-neurotoxic environment. In triculture, neuritic plaque formation with surrounding microglia was observed (FIG. 9D), similar to that observed in human AD postmortem brain sections. The addition of microglia to the co-culture system conferred a ˜25% basal protection to neuronal health and formed three-fold more Aβ plaques, suggesting that Aβ plaque formation and compaction may be neuroprotective (FIGS. 9H-91). When pro-inflammatory cytokines and Aβ42 oligomers were added to the triculture system, microglial-plaque association was increased and plaque formation increased six-fold, but there was a loss of neuroprotection (FIGS. 9D, and 9G-91). This suggests that microglial activation in response to Aβ might be beneficial in plaque compaction and neural protection, but over-activation could counteract these benefits through toxic microglial activities such as cytokine secretion.

These findings show that iPSC-derived neurons and microglia were capable of successfully modeling neuritic Aβ plaque formation surrounded by pTau positive dystrophic neurites, and encircled by microglia in close contact with the plaque; a key hallmark of AD pathology. These effects became exacerbated in a neuroinflammatory state, similar to that observed in late-stage human AD pathology.

### Example 5. Focused Small Molecule Screen Identifies DLK-JNK-cJun Pathway Inhibition Protects Human Neurons from Aβ Oligomer Toxicity

This example shows a focused small molecule drug screen, to further validate the AD model and demonstrate the screenablity of the platform. In particular, this example shows that DLKI-JNK-cJun pathway inhibition could protect human neurons from Aβ oligomer toxicity.

In order to demonstrate screenability of the system, and investigate whether observed AD pathology preserved molecular signaling events previously demonstrated in AD, a focused screen of 70 small molecules that have previously been shown to confer neuroprotection in various neurotoxic contexts, in addition to AD, was conducted (Table 1).

TABLE 1
Small molecules used in the focused screen.

| | | | | Bioactivity | | | |
|---|---|---|---|---|---|---|---|
| | | Cat. | | Information/ | | | Neuroprotective |
| No. | Name | No. | Vendor | Description | Dose | | citation |
| 1 | PD0325901 | 4192 | Tocris | MEK1/2 inhibitor; | 5 | mg/kg | Ku et al. 2018 |
| | | | | Prevents neuronal | | | |
| | | | | death caused by | | | |
| | | | | oxidative stress | | | |
| 2 | LM22A4 | 4607 | Tocris | TrkB agonist; | 0.001-1000 | nM | Massa et al. 2010 |
| | | | | Neurotrophic | | | |
| 3 | 7,8- | 3826 | Tocris | TrkB agonist. | 5 | mg/kg | Andero et al. 2012 |
| | Dihydroxyflavone | | | Neurotrophic | | | |
| 4 | LM11A 31 | 5046 | Tocris | p75NTR agonist; | 50 | mg/kg | Simmons et al. 2014 |
| | dihydrochloride | | | Increase survival | | | |
| | | | | signaling and inhibit | | | |
| | | | | amyloid-β-induced | | | |
| | | | | degenerative signaling | | | |
| 5 | (S)-(−)- | 1852 | Tocris | Myosin II ATPase | 1 | μM | Wang et al. 2017 |
| | Blebbistatin | | | inhibitor; Prevents | | | |
| | | | | oxidative | | | |
| | | | | stressinduced | | | |
| | | | | neuronal apoptosis | | | |
| 6 | BI-6C9 | Sc- | Santa Cruz | tBID inhibitor; | 10 | μM | Landshamer et al. |
| | | 210915A | Biotech | Protects from | | | 2008 |
| | | | | glutamate- | | | |
| | | | | induced neuronal | | | |
| | | | | death | | | |
| 7 | Bongkrekic acid | B6179 | Sigma | ANT inhibitor; | 4-16 | μg/kg | Muranyi et al. 2005 |
| | solution | | | Protects against | | | |
| | | | | NMDA receptor- | | | |
| | | | | mediated neuronal | | | |
| | | | | apoptosis | | | |
| 8 | Sodium butyrate | 3850 | Tocris | HDAC inhibitor; Anti- | 1.2 | g/kg | Kilgore et al. 2010 |
| | | | | inflammatory and | | | |
| | | | | neuroprotective | | | |
| 9 | Trichostatin A | 1406 | Tocris | HDAC inhibitor; Anti- | 5-10 | mg/kg | Fleiss et al. 2012 |
| | | | | inflammatory and | | | |
| | | | | neuroprotective | | | |
| 10 | Calpeptin | sc- | Santa Cruz | Calpain-2 inhibitor; | 2 | μM | Das et al. 2006 |
| | | 202516 | Biotech | Prevents neuronal | | | |
| | | | | apoptosis | | | |
| 11 | Kynurenic Acid | 3694 | Tocris | Nonspecific antagonist | 300 | mg/kg | Leib et al. 1996 |
| | Sodium Salt | | | of excitatory amino acid | | | |
| | | | | receptors; Protects | | | |
| | | | | from glutamate-induced | | | |
| | | | | neuronal death | | | |
| 12 | Necrostatin-1 | sc- | Santa Cruz | RIPK1 inhibitor; | 0.1-100 | μM | Degterev et al. 2005 |
| | | 200142 | Biotech | Block necroptosis and | | | |
| | | | | protect dopaminergic | | | |
| | | | | neurons | | | |
| 13 | BAX Inhibiting | B1436 | Sigma | BAX inhibitor; Inhibits | 5 μL, 5 | | Wang et al. 2010 |
| | Peptide V5 | | | neuronal apoptosis | mg/mL | | |
| 14 | Ivachtin | 2788-5 | BioVision | Caspase-3 inhibitor; | 0.5-50 | μM | Poksay et al. 2017 |
| | | | | Inhibits neuronal | | | |
| | | | | apoptosis | | | |
| 15 | Cdk2 Inhibitor II | 219445 | Calbiochem | CDK2 inhibitor; | 4 | μM | Ye et al. 2010 |
| | | | | Inhibits neuronal | | | |
| | | | | apoptosis triggered by | | | |
| | | | | inappropriate | | | |
| | | | | activation of CDK | | | |
| 16 | SB 218078 | 2560 | Tocris | Chk1 inhibitor | 5 | μM | Gonzalez et al. 2015 |
| 17 | PD 0332991 | 4786 | Tocris | CDK inhibitor; | 100 | mg/kg | Marathe et al. 2015 |
| | isethionate | | | Inhibits neuronal | | | |
| | | | | apoptosis triggered by | | | |
| | | | | inappropriate | | | |
| | | | | activation of CDK | | | |
| 18 | Purvalanol A | 1580 | Tocris | CDK inhibitor; | 75 | nM | Kuruva et al. 2016 |
| | | | | Inhibits neuronal | | | |
| | | | | apoptosis triggered by | | | |
| | | | | inappropriate | | | |
| | | | | activation of CDK | | | |
| 19 | Olomoucine | 1284 | Tocris | CDK inhibitor; | 1-100 | μM | Di Giovanni et al. |
| | | | | Inhibits neuronal | | | 2005 |
| | | | | apoptosis triggered by | | | |
| | | | | inappropriate | | | |
| | | | | activation of CDK | | | |
| 20 | GW8510 | G7791 | Sigma | CDK2 inhibitor; | 1-10 | μM | Johnson et al. 2005 |
| | | | | Inhibits neuronal | | | |
| | | | | apoptosis triggered by | | | |
| | | | | Inappropriate | | | |
| | | | | activation of CDK | | | |
| 21 | SB216763 | S1075 | Selleckchem | GSK-3β Inhibitor; | 3 | μM | Liang and Chuang |
| | | | | Protects against axon | | | 2006 |
| | | | | degeneration | | | |
| 22 | TDZD-8 | ALX- | Enzo | GSK-3β Inhibitor; | 3.3 & 10 | μM | Martinez et al. 2002 |
| | | 270-354- | | Protects against axon | | | |
| | | M005 | | degeneration | | | |
| 23 | IM-12 | SML0084 | Sigma | GSK-3β Inhibitor; | 1 | μM | Shan et al. 2017 |
| | | | | Protects against axon | | | |
| | | | | degeneration | | | |
| 24 | CHIR 99021 | 4953 | Tocris | GSK-3β Inhibitor; | 3.1-25 | mg/kg | Pan et al. 2011 |
| | trihydrochloride | | | Protects against axon | | | |
| | | | | degeneration | | | |
| 25 | Saracatinib | S1006 | Selleckchem | Fyn inhibitor; | 2-1000 | nM | Nygaard, Dyck and |
| | (AZD0530) | | | Neuroprotective | | | Strittmatter 2014 |
| 26 | SU6656 | S7774 | Selleckchem | Fyn inhibitor; | 5 | μM | Johnson et al. 2005 |
| | | | | Neuroprotective | | | |
| 27 | sun11602 | 4826 | Tocris | Fyn inhibitor; | 1 & 3 | μM | Murayama et al. 2013 |
| | | | | Neuroprotective | | | |
| 28 | GM 6001 | 2983 | Tocris | Matrix metalloproteinase | 5 | μg/mouse | Shichi et al. 2011 |
| | | | | inhibitor | | | |
| 29 | Indirubin-3′- | 1813 | Tocris | GSK3β and CDK | 0.04-20 | μM | Rudhard et al. 2015 |
| | monoxime | | | inhibitor; Protects | | | |
| | | | | against axon | | | |
| | | | | degeneration; Anti- | | | |
| | | | | apoptotic and | | | |
| | | | | neuroprotective | | | |
| 30 | AS601245 | ALX- | Tocris | JNK inhibitor.; Anti- | 0.04-20 | μM | Rudhard et al. 2015 |
| | | 270-443- | | inflammatory | | | |
| | | M005 | | And neuroprotective | | | |
| 31 | P7C3 | 4076 | Tocris | NAMPT activator; | 5-40 mg/kg + | | Pieper et al. 2010 |
| | | | | Proneurogenic and | J33F33:I33F33:H33 | | |
| | | | | neuroprotective | | | |
| 32 | Daunorubicin | 1467 | Tocris | increases gangliosides | 0.04-20 | μM | Rudhard et al. 2015 |
| | hydrochloride | | | (especially GQ1b) | | | |
| | | | | expression in | | | |
| | | | | differentiating | | | |
| | | | | neuronal cells | | | |
| 33 | MG-132 | 1748 | Tocris | Calpain and protease | 0.04-20 | μM | Rudhard et al. 2015 |
| | | | | inhibitor | | | |
| 34 | Capsazepine | 0464 | Tocris | Vanilloid receptor | 0.04-20 | μM | Rudhard et al. 2015 |
| | | | | antagonist; Anti- | | | |
| | | | | inflammatory | | | |
| 35 | SU 11248 | 3768 | Tocris | Inhibitor of multiple | 0.04-20 | μM | Rudhard et al. 2015 |
| | | | | receptor transduction | | | |
| | | | | kinases | | | |
| 36 | SU 6668 | 3335 | Tocris | PDGFR, VEGFR and | 0.04-20 | μM | Rudhard et al. 2015 |
| | | | | FGFR inhibitor | | | |
| 37 | Ac-Leu-Leu-Nle- | BML- | Tocris | calpain I, calpain II, | 0.04-20 | μM | Rudhard et al. 2015 |
| | CHO | P120- | | cathepsin L inhibitor; | | | |
| | | 0005 | | Prevents neuronal | | | |
| | | | | apoptosis | | | |
| 38 | MDL 28170 | 1146 | Tocris | Calpain and Cathepsin | 0.04-20 | μM | Rudhard et al. 2015 |
| | | | | B inhibitor; Prevents | | | |
| | | | | neuronal apoptosis | | | |
| 39 | SB 239063 | 1962 | Tocris | p38 MAPK inhibitor; | 0.04-20 | μM | Rudhard et al. 2015 |
| | | | | Protects against axon | | | |
| | | | | degeneration | | | |
| 40 | BAY 11-7082 | 1744 | Tocris | NF-κB inhibitor; Anti- | 0.04-20 | μM | Rudhard et al. 2015 |
| | | | | inflammatory | | | |
| | | | | And neuroprotective | | | |
| 41 | Luteolin | 2874 | Tocris | Anti-inflammatory, | 0.04-20 | μM | Rudhard et al. 2015 |
| | | | | antioxidant | | | |
| | | | | and free radical | | | |
| | | | | scavenger. Induces Nrf2 | | | |
| | | | | and inhibits caspase-3 | | | |
| | | | | activation | | | |
| 42 | Teniposide | SML0609 | Sigma | Topoisomerase II | 0.04-20 | μM | Rudhard et al. 2015 |
| | | | | inhibitor; Inhibits | | | |
| | | | | DNA synthesis | | | |
| 43 | 2-TEDC | 0645 | Tocris | 5-, 12-, -15- | 0.04-20 | μM | Rudhard et al. 2015 |
| | | | | lipoxygenase inhibitor; | | | |
| | | | | Protects against axon | | | |
| | | | | degeneration | | | |
| 44 | SB 415286 | 1617 | Tocris | GSK-3β inhibitor; | 0.04-20 | μM | Rudhard et al. 2015 |
| | | | | Protects against axon | | | |
| | | | | degeneration | | | |
| 45 | FK 506 | 3631 | Tocris | Calcineurin inhibitor; | 0.5-1 | mg/kg | Sierra-Paredes and |
| | | | | Neuroprotective | | | Sierra-Marcuno |
| 46 | STEARDA | 2204 | Tocris | 5-LO (5-lipoxygenase) | 0.04-20 | μM | Rudhard et al. 2015 |
| | | | | inhibitor; Protects | | | |
| | | | | against axon degeneration | | | |
| 47 | Arctigenin | 1777 | Tocris | MKK1 and IKBa inhibitor, | 0.04-20 | μM | Rudhard et al. 2015 |
| | | | | neuroprotective by | | | |
| | | | | binding to kainate | | | |
| | | | | receptors | | | |
| 48 | Lycorine | HY- | MedChemExpress | p21CIP1/WAF1 | 0.04-20 | μM | Rudhard et al. 2015 |
| | hydrochloride | N0289 | | activator; Inhibits | | | |
| | | | | caspase-3 and prevents | | | |
| | | | | apoptosis | | | |
| 49 | NKH 477 | 1603 | Tocris | Adenylyl cyclase activator | 0.04-20 | μM | Rudhard et al. 2015 |
| 50 | Demeclocycline | HY- | MedChemExpress | Calpain inhibitor; | 0.04-20 | μM | Rudhard et al. 2015 |
| | hydrochloride | 17560 | | Prevents neuronal | | | |
| | | | | apoptosis | | | |
| 51 | PDI Inhibitor | SML0021 | Sigma | PDI Inhibitor. Prevents | 0.5-100 | μM | Hoffstrom et al. 2010 |
| | 16F16 | | | apoptosis induced by | | | |
| | | | | misfolded proteins | | | |
| 52 | JWH 015 | 1341 | Tocris | cannabinoid (CB2) | 0.04-20 | μM | Rudhard et al. 2015 |
| | | | | receptor agonist | | | |
| 53 | GW 5074 | 1381 | Tocris | cRaf1 kinase inhibitor | 0.04-20 | μM | Rudhard et al. 2015 |
| 54 | GBR 12783 | 0513 | Tocris | Dopamine uptake inhibitor | 0.04-20 | μM | Rudhard et al. 2015 |
| | dihydrochloride | | | | | | |
| 55 | Baicalein | 1761 | Tocris | 5-, and 12-lipoxygenase | 0.04-20 | μM | Rudhard et al. 2015 |
| | | | | Inhibitor; Protects | | | |
| | | | | against axon degeneration | | | |
| 56 | GNE-3511 | HY- | MedChemExpress | DLK inhibitor; Protects | 0.04-20 | μM | Pichon et al |
| | | 12947 | | against neuronal and | | | |
| | | | | synaptic loss | | | |
| 57 | Edaravone | 0786 | Tocris | Free radical scavenger; | 1 & 3 | mg/kg | Kawasaki et al. 2007 |
| | | | | Protects from ROS-induced | | | |
| | | | | neurotoxicity | | | |
| 58 | C 646 | 4200 | Tocris | p300/CBP (HAT) inhibitor | 20 | μM | Formisano et al. 2015 |
| 59 | Zileuton | 3308 | Tocris | 5-lipoxygenase (5-LOX) | 0.04-20 | μM | Rudhard et al. 2015 |
| | | | | inhibitor | | | |
| 60 | TRO 19622 | 2906 | Tocris | Mitochondrial | 0.1-10 | μM | Bordet et al. 2007 |
| | | | | permeability transition | | | |
| | | | | pore inhibitor; | | | |
| | | | | Neuroprotective | | | |
| 61 | Resveratrol | 1418 | Tocris | Cyclooxygenase | 0.1-50 | μM | Bastianetto, Menard, |
| | | | | inhibitor. Antioxidant, | | | and Quirion |
| | | | | neuroprotective | | | |
| | | | | against Aβ-related | | | |
| | | | | neurotoxicity | | | |
| 62 | IU1 | 4088 | Tocris | Deubiquitinating | 400 | μg/kg | Min et al. 2017 |
| | | | | enzyme USP14 | | | |
| | | | | inhibitor; Reduce | | | |
| | | | | protein aggregates and | | | |
| | | | | protects from neuronal | | | |
| | | | | loss | | | |
| 63 | ISR Inhibitor, | 509584 | Calbiochem | Integrated stress | 0.5-100 | nM | Hosoi et al. 2016 |
| | ISRIB | | | response (ISR) | | | |
| | | | | Inhibitor; Prevents | | | |
| | | | | neuronal cell death | | | |
| | | | | through inhibition of | | | |
| | | | | amyloid β-induced | | | |
| | | | | ATF4 induction | | | |
| 64 | CTPB | ALX- | Enzo Life | p300 histone | 0.5-200 | μM | Hegarty et al. 2016 |
| | | 420-033- | Sciences | acetyltransferase | | | |
| | | M005 | | (HAT) activator | | | |
| 65 | Fluorobexarotene | 4064 | Tocris | RXR agonist; Stimulates | 20 | mg/kg | Bachmeier et al. 2013 |
| | | | | the metabolic clearance | | | |
| | | | | of Aβ | | | |
| 66 | AK 7 | 4754 | Tocris | SIRT2 inhibitor; | 10, 20, & 30 | | Chopra et al. 2012 |
| | | | | Neuroprotective in | mg/kg | | |
| | | | | Huntington's and | | | |
| | | | | Parkinson's murine | | | |
| | | | | models | | | |
| 67 | Epicatechin | HY- | MedChemExpress | Anti-oxidant and anti- | 10 | mg/kg | Pinto et al 2015 |
| | | N0001 | | inflammatory; | | | |
| | | | | Neuroprotective | | | |
| 68 | Guggulsterone | 2013 | Tocris | Steroid receptors | 30 & 60 | mg/kg | Chen, Huang, Ding |
| | | | | antagonist; Anti- | | | 2016 |
| | | | | inflammatory in | | | |
| | | | | microglia | | | |
| 69 | Clusterin Protein | 2937-HS | R&D systems | Prevents Aβ | | | Pucci et al. 2008 |
| | | | | aggregation and | | | |
| | | | | Fibrillization; Anti- | | | |
| | | | | apoptotic | | | |
| 70 | Neuropathiazol | 5186 | Tocris | Neuronal differentiation | 0.6-5 | μM | Wurdak et al. 2010 |
| | | | | inducer in | | | |
| | | | | hippocampal neural | | | |
| | | | | progenitors | | | |

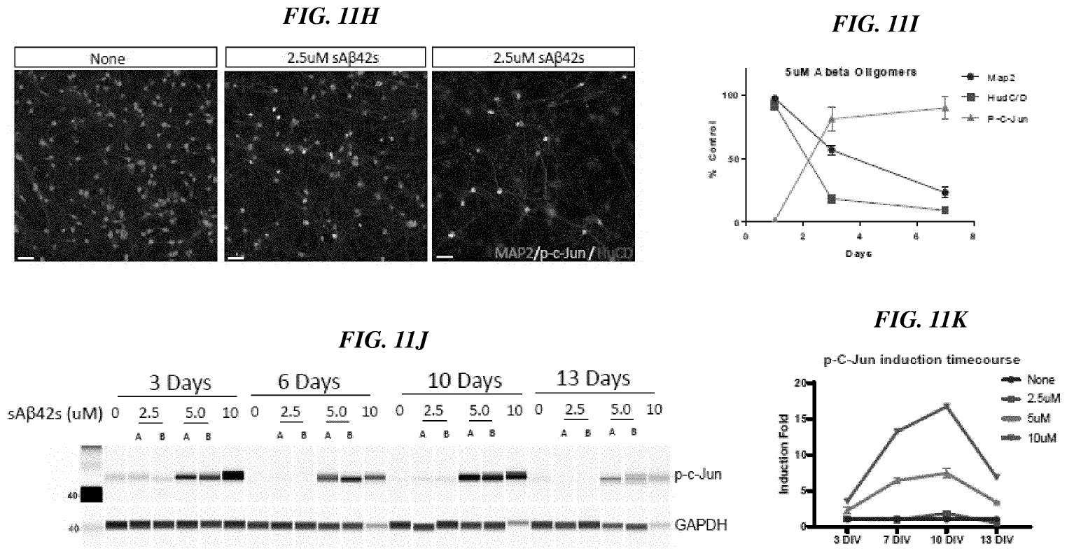

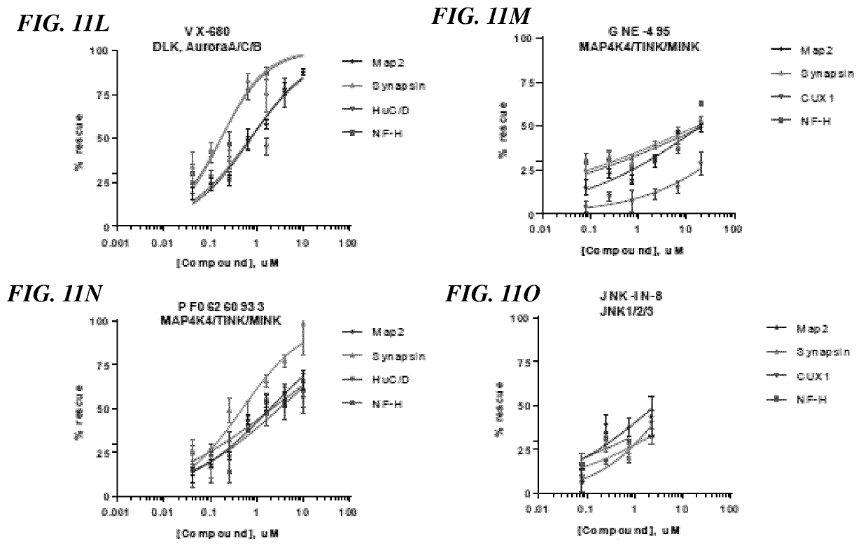

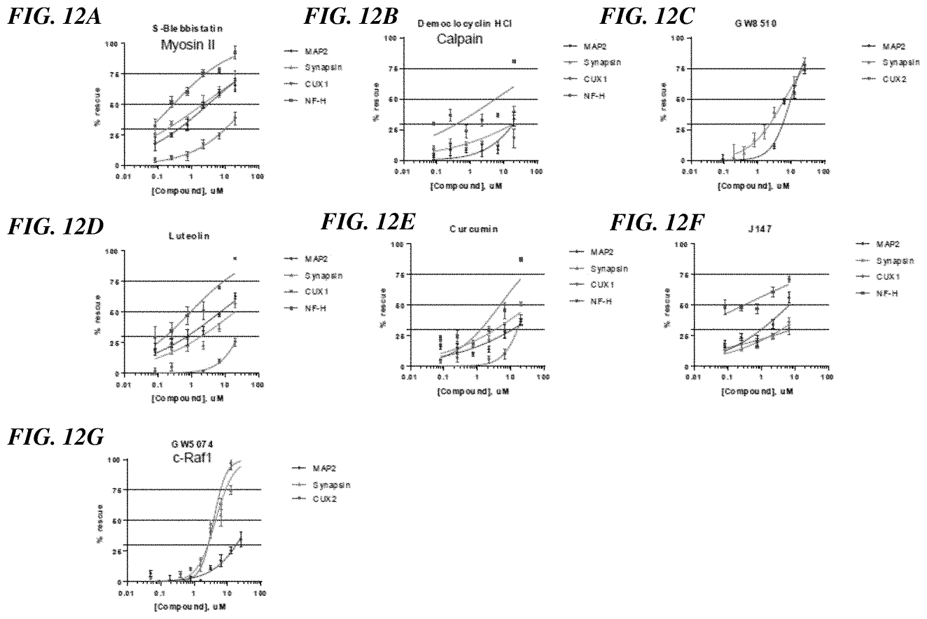

In double (neuron, astrocyte) and triple (neuron, astrocyte, microglia) cultures, each small molecule listed in Table 1 was tested in the AD model paradigm at up to four concentrations. Molecules that characterized ≥30% rescue in any of the four measurements of dendrites area (MAP2), synapses count (synapsin 1/2), or cell count (CUX2), or axon area (Beta III Tubulin; “BT3”)) were classified as hits (FIGS. 11A-11D, Table 2, and Table 3).

TABLE 2
Focused screen results.

| Number | | Known Axon | | | | | |
|---|---|---|---|---|---|---|---|
| (from | | Protection | Conc. | % MAP2 | % Cux2 | % Synapse | % B3T |
| Table 1) | Compound | Effect? | (μM) | rescue | rescue | rescue | rescue |
| 5 | (S)-(−)- | No | 50 | 34% | 23% | 12% | 49% |
| | Blebbistatin | | 25 | 34% | 5% | 13% | 38% |
| | | | 12.5 | 25% | 3% | 15% | 34% |
| | | | 6.25 | 21% | 10% | 19% | 41% |
| 43 | 2-TEDC | Yes | 50 | 22% | 16% | 10% | 31% |
| 3 | 7,8-Dihydroxyflavone | No | 50 | 6% | 42% | −2% | −14% |
| 37 | Ac-Leu-Leu- | Yes | 50 | 23% | 78% | 2% | 0% |
| | Nle-CHO | | 25 | 20% | 87% | −5% | −11% |
| | | | 12.5 | 18% | 87% | −4% | 0% |
| | | | 6.25 | 31% | 85% | 0% | 14% |
| 10 | Calpeptin | Yes | 50 | 36% | 96% | 15% | 20% |
| | | | 25 | 39% | 91% | 9% | 7% |
| | | | 12.5 | 27% | 79% | 11% | 4% |
| | | | 6.25 | 22% | 70% | 6% | 9% |
| 50 | Demeclocydine | Yes | 50 | 39% | 33% | 27% | 63% |
| | hydrochloride | | 25 | 23% | 6% | 15% | 35% |
| 38 | MDL 28170 | Yes | 50 | 26% | 57% | 23% | 47% |
| | | | 25 | 36% | 84% | 11% | 25% |
| | | | 12.5 | 33% | 74% | 9% | 22% |
| | | | 6.25 | 33% | 81% | 10% | 18% |
| 66 | AK 7 | No | 50 | −17% | 0% | −5% | 42% |
| 30 | AS601245 | Yes | 50 | 64% | 123% | 19% | −16% |
| | | | 25 | 73% | 32% | 36% | 11% |
| | | | 12.5 | 67% | 13% | 57% | 20% |
| | | | 6.25 | 49% | 9% | 37% | 14% |
| 40 | BAY 11-7082 | Yes | 25 | 17% | 82% | −13% | 14% |
| | | | 12.5 | 36% | 85% | −3% | 31% |
| | | | 6.25 | 34% | 70% | −2% | 38% |
| 41 | Luteolin | Yes | 50 | 55% | 19% | 37% | 66% |
| | | | 25 | 26% | 12% | 13% | 29% |
| 33 | MG-132 | Yes | 50 | 25% | 90% | 4% | 57% |
| | | | 25 | 26% | 91% | 8% | 30% |
| | | | 12.5 | 24% | 87% | 4% | 7% |
| | | | 6.25 | 18% | 80% | −6% | −3% |
| 58 | C 646 | No | 50 | 54% | 25% | 19% | 49% |
| 32 | Daunorubicin | Yes | 50 | −124% | −39% | −27% | 101% |
| | hydrochloride | | 25 | −122% | −23% | −27% | 73% |
| 56 | GNE-3511 | Yes | 50 | 87% | 81% | 47% | 58% |
| | | | 25 | 49% | 36% | 14% | 17% |
| | | | 6.25 | 32% | 18% | 7% | 15% |
| 53 | GW 5074 | Yes | 50 | 54% | 162% | 8% | 48% |
| | | | 25 | 19% | 115% | 8% | 14% |
| | | | 12.5 | 12% | 76% | 6% | 9% |
| | | | 6.25 | 17% | 95% | 8% | 16% |
| 20 | GW8510 | No | 50 | 52% | −22% | 49% | 47% |
| | | | 25 | 37% | −18% | 38% | 31% |
| | | | 12.5 | 37% | 16% | 38% | 20% |
| | | | 6.25 | 48% | −1% | 52% | 32% |
| 17 | PD 0332991 isethionate | No | 50 | −97% | −37% | −15% | 69% |
| 12 | Necrostatin-1 | Yes | 50 | 37% | 10% | 15% | 11% |
| 49 | NKH 477 | Yes | 50 | 44% | 45% | −5% | 38% |
| | | | 25 | 43% | 21% | −4% | 35% |
| | | | 12.5 | 45% | 24% | 5% | 38% |
| | | | 6.25 | 39% | 21% | 7% | 38% |
| 51 | PDI Inhibitor | No | 50 | −12% | 34% | −13% | −22% |
| | 16F16 | | 25 | −10% | 66% | −8% | −46% |
| | | | 12.5 | 1% | 45% | −1% | 3% |
| | | | 6.25 | 6% | 46% | 5% | 27% |
| 25 | Saracatinib | No | 25 | 65% | 18% | 10% | 30% |
| | (AZD0530) | | 12.5 | 71% | 13% | 34% | 56% |
| | | | 6.25 | 61% | 7% | 32% | 62% |
| 26 | SU6656 | No | 50 | 3% | 106% | 11% | −188% |
| | | | 25 | 3% | 126% | 15% | −37% |
| | | | 12.5 | 4% | 120% | 11% | 3% |
| | | | 6.25 | 5% | 101% | 13% | 4% |
| 16 | SB 218078 | No | 50 | −92% | −39% | −15% | 72% |
| 44 | SB 415286 | Yes | 50 | 19% | −13% | 12% | 39% |
| 35 | SU 11248 | Yes | 25 | 16% | 59% | 4% | −19% |
| | | | 50 | 7% | 115% | 21% | 4% |

TABLE 3
Focused screen results: triple culture.

| Number | | Known Axon | | | | |
|---|---|---|---|---|---|---|
| (from | | Protection | Conc. | % MAP2 | % Synapse | % B3T |
| Table 1) | Compound | Effect? | (μM) | rescue | rescue | rescue |
| 60 | TRO 19622 | No | 12.50 | −36 | −13% | 33% |
| | | | 3.13 | −25% | −10% | 34% |
| 66 | AK 7 | No | 50.00 | 28% | 17% | 56% |
| | | | 12.50 | 53% | 42% | 81% |
| | | | 3.13 | 18% | 13% | 77% |
| | | | 0.78 | 1% | 3% | 79% |
| 61 | Resveratrol | No | 50.00 | 84% | 78% | 78% |
| | | | 12.50 | 57% | 49% | 54% |
| | | | 3.13 | 33% | 27% | 30% |
| | | | 0.78 | 33% | 24% | 27% |
| 67 | Epicatechin | No | 50.00 | 28% | 20% | 30% |
| 62 | IU1 | No | 12.50 | 32% | 23% | 41% |
| 68 | Guggulsterone | No | 50.00 | 19% | 17% | 36% |
| 63 | ISR Inhibitor, ISRIB | No | 50.00 | 31% | 21% | 22% |
| 58 | C 646 | No | 50.00 | 37% | 39% | 53% |
| | | | 12.50 | 41% | 38% | 42% |
| | | | 0.78 | 37% | 28% | 27% |
| 70 | Neuropathiazol | No | 50.00 | 27% | 28% | 35% |
| | | | 12.50 | 44% | 42% | 35% |
| 65 | Fluorobexarotene | No | 12.50 | 40% | 33% | 33% |
| | | | 3.13 | 43% | 34% | 50% |
| 56 | GNE-3511 | Yes | 50.00 | 66% | 56% | 65% |
| | | | 12.50 | 102% | 81% | 82% |
| | | | 3.13 | 95% | 77% | 81% |
| | | | 0.78 | 91% | 79% | 85% |
| 37 | Ac-Leu-Leu-Nle-CHO | Yes | 12.50 | 48% | 18% | 44% |
| | | | 3.13 | 55% | 38% | 57% |
| | | | 0.78 | 21% | 14% | 38% |
| 43 | 2-TEDC | Yes | 50.00 | 48% | 40% | 73% |
| | | | 12.50 | 25% | 17% | 43% |
| | | | 3.13 | 22% | 15% | 40% |
| 49 | NKH 477 | Yes | 50.00 | 53% | 38% | 81% |
| | | | 12.50 | 9% | 8% | 49% |
| | | | 3.13 | 17% | 12% | 42% |
| 38 | MDL 28170 | Yes | 50.00 | 47% | 41% | 73% |
| | | | 12.50 | 31% | 8% | 33% |
| | | | 0.78 | 19% | 10% | 30% |
| 44 | SB 415286 | Yes | 50.00 | 42% | 40% | 78% |
| 45 | FK 506 | No | 12.50 | 34% | 34% | 52% |
| 51 | PDI Inhibitor 16F16 | No | 50.00 | −5% | −1% | 42% |
| | | | 12.50 | −6% | 8% | 43% |
| | | | 3.13 | 44% | 43% | 71% |
| 40 | BAY 11-7082 | Yes | 50.00 | 21% | −1% | 44% |
| 46 | STEARDA | No | 12.50 | 24% | 21% | 33% |
| | | | 3.13 | 28% | 24% | 33% |
| | | | 0.78 | 30% | 24% | 27% |
| 52 | JWH 015 | No | 50.00 | 38% | 26% | 42% |
| | | | 12.50 | 50% | 43% | 60% |
| 41 | Luteolin | Yes | 50.00 | 73% | 75% | 91% |
| 42 | Teniposide | Yes | 50.00 | −19% | 3% | 52% |
| | | | 12.50 | −17% | 3% | 50% |
| | | | 3.13 | −10% | 5% | 42% |
| | | | 0.78 | −7% | 1% | 35% |
| 48 | Lycorine hydrochloride | Yes | 50.00 | 88% | 75% | 79% |
| | | | 12.50 | 93% | 77% | 83% |
| | | | 3.13 | 99% | 80% | 77% |
| | | | 0.78 | 57% | 43% | 37% |
| 19 | Olomoucine | No | 50.00 | −8% | −15% | 69% |
| 25 | Saracatinib (AZD0530) | No | 12.50 | −12% | −2% | 35% |
| | | | 3.13 | 73% | 65% | 99% |
| | | | 0.78 | 72% | 65% | 99% |
| 31 | P7C3 | No | 50.00 | −8% | 2% | 43% |
| | | | 3.13 | 2% | 3% | 30% |
| 20 | GW8510 | No | 50.00 | −8% | −15% | 63% |
| | | | 12.50 | 28% | 32% | 68% |
| | | | 3.13 | 15% | 25% | 70% |
| | | | 0.78 | −4% | 8% | 45% |
| 26 | SU6656 | No | 50.00 | 40% | 36% | 48% |
| | | | 12.50 | 53% | 49% | 71% |
| | | | 3.13 | 72% | 64% | 92% |
| | | | 0.78 | 58% | 51% | 80% |
| 32 | Daunorubicin hydrochloride | Yes | 0.78 | 74% | 52% | 68% |
| 21 | SB216763 | No | 50.00 | −24% | −15% | 46% |
| 27 | sun11602 | No | 50.00 | 14% | 11% | 47% |
| | | | 12.50 | 19% | 15% | 43% |
| | | | 3.13 | 10% | 11% | 33% |
| 33 | MG-132 | Yes | 50.00 | 25% | −11% | 46% |
| | | | 12.50 | 18% | −7% | 35% |
| 22 | TDZD-8 | No | 50.00 | −29% | −8% | 54% |
| 34 | Capsazepine | Yes | 12.50 | 44% | 44% | 50% |
| 29 | Indinibin-3′-monoxime | Yes | 50.00 | 23% | 34% | 49% |
| | | | 12.50 | 37% | 44% | 74% |
| | | | 3.13 | 6% | 9% | 31% |
| 35 | SU 11248 | Yes | 3.13 | 88% | 77% | 74% |
| | | | 0.78 | 73% | 68% | 69% |
| 24 | CHIR 99021 trihydrochloride | No | 50.00 | 58% | 46% | 66% |
| 30 | AS601245 | Yes | 50.00 | 95% | 45% | 44% |
| | | | 12.50 | 74% | 26% | 26% |
| | | | 3.13 | 54% | 17% | 18% |
| 36 | SU 6668 | No | 50.00 | 80% | 75% | 77% |
| | | | 3.13 | 31% | 26% | 34% |
| 10 | Calpeptin | Yes | 50.00 | 34% | 34% | 66% |
| 16 | SB 218078 | No | 50.00 | 53% | 19% | −11% |
| | | | 0.78 | 10% | 14% | 38% |
| 6 | BI-6C9 | No | 50.00 | 58% | 55% | 65% |
| 1 | PD0325901 | No | 50.00 | 27% | 27% | 59% |
| | | | 12.50 | 8% | 8% | 43% |
| | | | 3.13 | 23% | 21% | 52% |
| | | | 0.78 | 19% | 15% | 48% |
| 13 | BAX Inhibiting Peptide V5 | No | 12.50 | −6% | 14% | 32% |
| 9 | | No | 50.00 | −13% | 1% | 41% |
| | Trichostatin A | | 12.50 | −18% | 2% | 31% |
| 5 | (S)-(−)-Blebbistatin | No | 50.00 | 24% | 20% | 36% |
| 17 | PD 0332991 isethionate | No | 50.00 | −79% | −29% | 90% |
| 18 | Purvalanol A | No | 3.13 | 15% | 24% | 33% |

Overlapping hits from double and triple cultures were observed, indicating those small molecules promising top hits. Nine hits from both screens were confirmed with IC50 curves in double culture, including inhibitors of well-known active kinases in AD such as DLKi, Indirubin-3′-monoxime (GSK3β and CDK5 inhibitor), and AZD0530 (Fyn inhibitor). Importantly, GSK3, CDK5, and Fyn are known Tau-acting kinases, and two natural products, luteolin and curcumin, have shown to provide a protective effect in AD. Curcumin and its derivative, J147, were validated with IC50 curves (FIGS. 12A-12G, Table 2, Table 3). In addition, multiple calpain inhibitors from the primary screens were identified, and demeclocycline HCl was validated with an IC50 curve (FIGS. 11E-11G, FIGS. 12A-12G, Table 2 Table 3).

Since DLK inhibition was the most protective compound, and JNK inhibition (AS601245) was also protective to a lesser extent in focused screens in both double and triple cultures, the next step was to validate this pathway and investigate whether the DLK-JNK-cJun signaling pathway is activated in the AD model. Induction of the phosphorylation of cJun was observed when human neurons were treated with Aβ42 oligomer (FIG. 11J). This effect was persistent (up to 13 days), and increased in a dose-dependent manner with soluble Aβ42 species concentration (FIGS. 11H-11K).

To further validate this pathway, several known inhibitors of kinases in the DLK signaling pathway were tested to determine whether they could also be neuroprotective in the AD model. Inhibition with VX-680 (a different DLK inhibitor), GNE-495 (MAP4K4 inhibitor upstream of DLK), PF06260933 (a different MAP4K4 inhibitor), and JNK-IN-8 (JNK1/2/3 inhibitor), all conferred neuronal protection against Aβ in a dose-dependent manner (FIGS. 11L-11O).

The focused screen resulted in the identification and validation of several compounds targeting proteins in several known mechanisms in human AD, such as DLK, GSK3, CDK5, and Fyn kinase, all of which are current pathways of interest in drug development. The results show that the in vitro human neuron-based AD model not only demonstrated phenotypes of AD not previously seen in vitro, but also recapitulated important pathological signaling events that contributed to these observed phenotypes. Overall, the validation of known molecular signaling pathways previously shown to be important drug targets suggests that the in vitro human neuron AD model is a translationally relevant molecular neurobiology, and could be used as a high throughput screening tool that can facilitate target discovery and characterization and larger drug development efforts.

### Example 6. Cellular Mechanism of Microglia Amyloid Plaque Formation

This example shows the cellular process of microglial plaque formation in the AD model system.

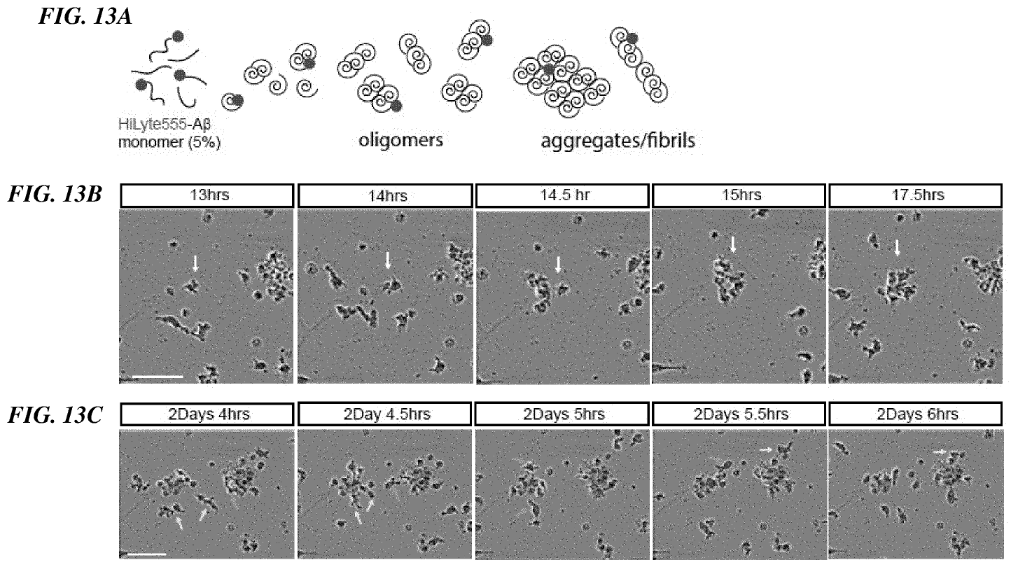

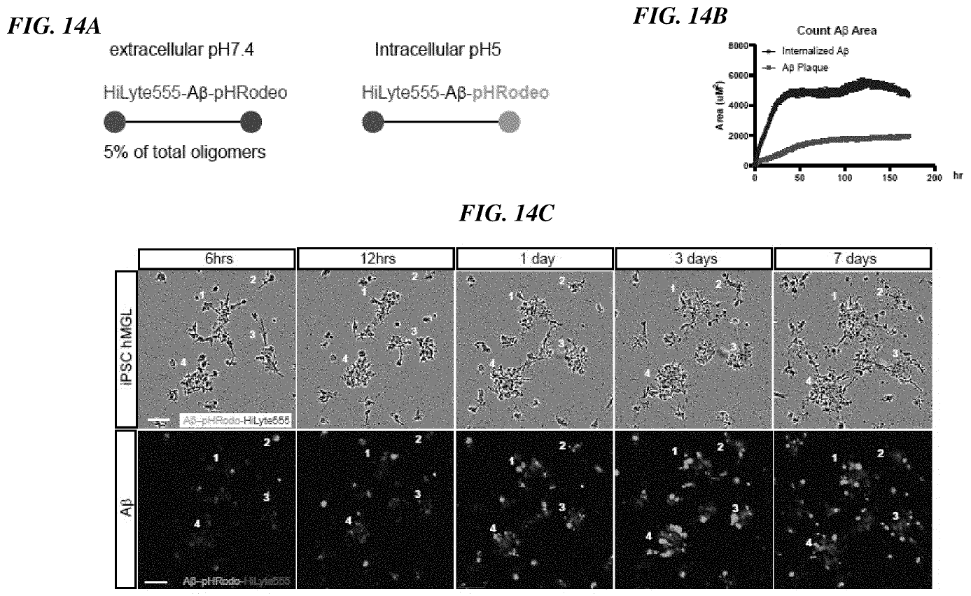

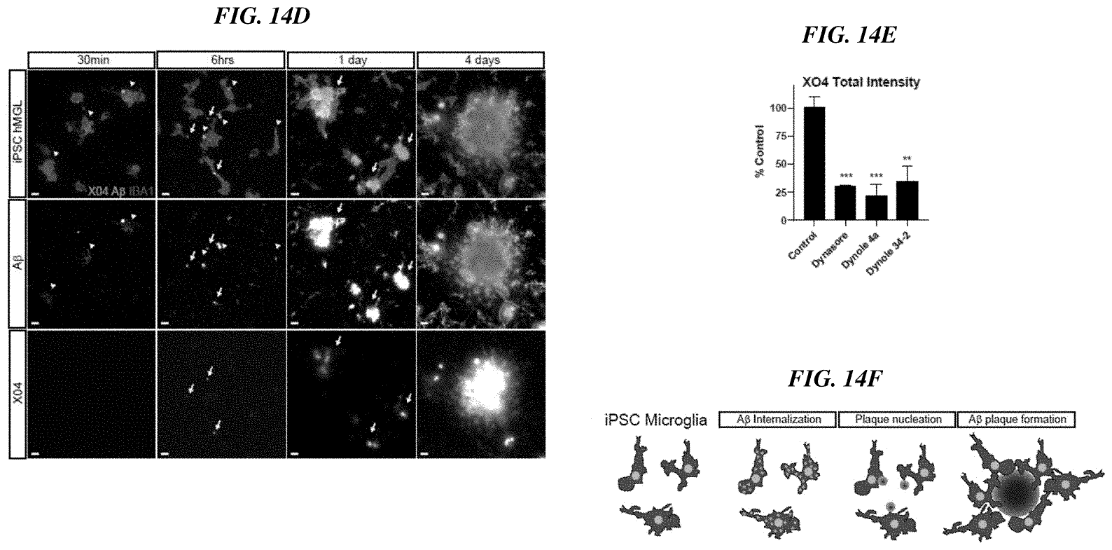

Since the AD model system recapitulated amyloid plaque formation robustly, the next step was to understand the cellular process of plaque formation. To observe plaque formation by microglia, time-lapse studies at 30 minutes intervals for 7 days with microglia were conducted, and compared to a similar cell type (e.g., human CD14-derived macrophages). HiLytem-555 labeled Aβ42 monomers were used to generate red soluble Aβ42 species (FIG. 13A). Microglia were uniquely highly motile during and after plaque formation compared with the macrophages, extending and retracting their processes and moving dynamically in and out of plaques (FIG. 7C). Aβ plaque formation appeared to form extracellularly within clusters of microglial cells and grew larger (FIG. 13B). In contrast, human macrophages were relatively stationary and continuously internalized red Aβ42 oligomers. pHrodo® green dye was then incorporated into HiLytem-555 labeled oligomers, to allow for a concurrent observation of Aβ42 internalization (green) and plaque formation (red) (FIG. 14A). Microglia first internalized Aβs prior to plaque formation (FIGS. 14B-14C). These results taken together suggest that microglia may internalize soluble Aβ42 species first, and then exocytose and package them as plaque structures (FIG. 10).

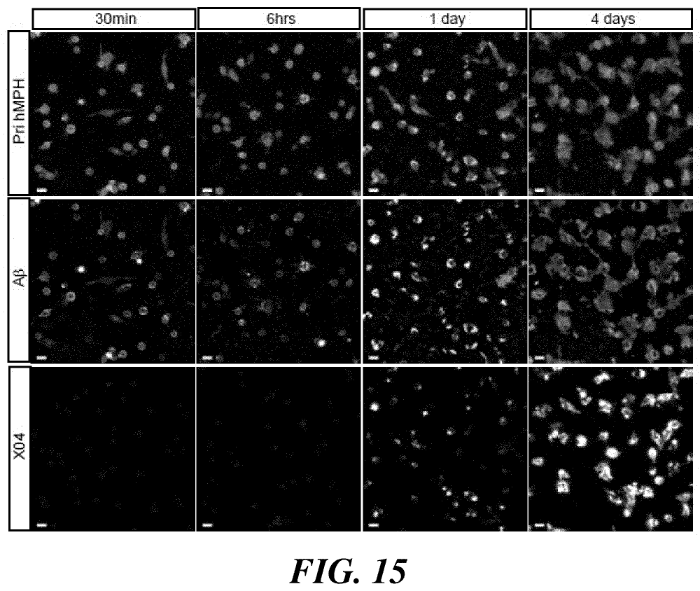

To further confirm time-lapsed result, an immunostaining time-course study was performed. Microglia took up Aβs within 30 minutes (FIG. 14D). After 6 hours, small internalized puncta disappeared and a larger, faintly X04 positive, Aβ42 aggregate appeared at the edge near each cell (FIG. 14D). After 1 day, larger Aβ42 aggregates with higher X04 staining intensity were seen next to microglia, and additional microglia began to surround these aggregates. At 4 days X04 dye positive plaque structures were present with surrounding microglia. This behavior appeared unique to microglial cells, as human macrophages appear to continuously internalize Aps, and then appear to die (FIG. 15). Finally, to test if endocytosis is involved in this process, microglia were treated with dynamin inhibitors which reduce endocytosis. There was a 75% reduction of plaque formation upon treatment with dynamin, indicating that microglia internalization of Aβ42 is critical for amyloid plaque formation (FIG. 14E).

### Example 7. Modeling AD Progression and Anti-Aβ Antibody Intervention

This example shows a model of the progression of AD and continuous Aβ exposure, which may be modulated to generate a progressive AD disease model with precise temporal control of neurodegeneration speed. In particular, this example shows the mechanism of action of a large molecule therapeutic anti-Aβ antibody, and further optimization of the AD model to use 8 fold less Aβs to simulate AD progression and evaluate anti-Aβ antibody intervention.

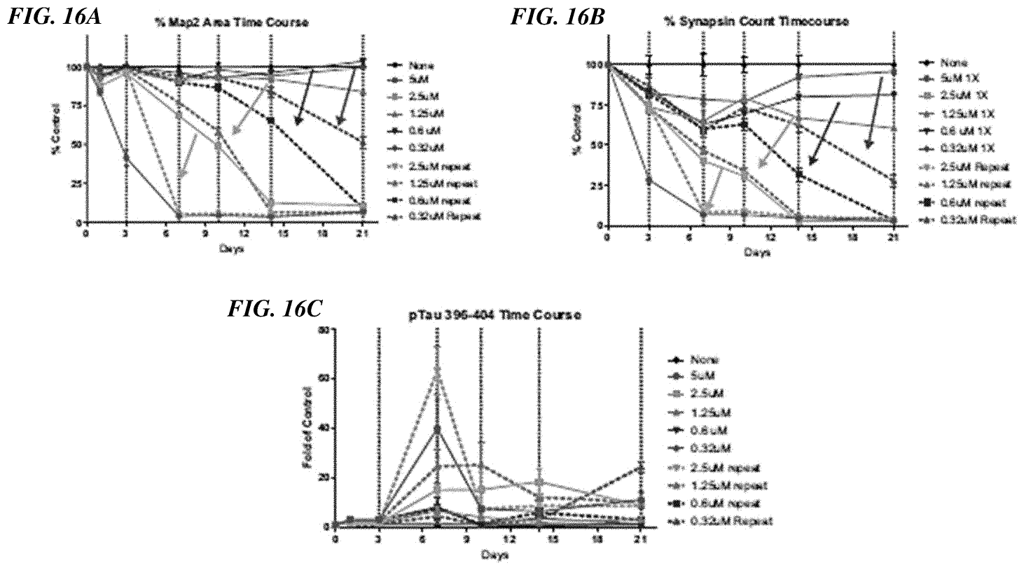

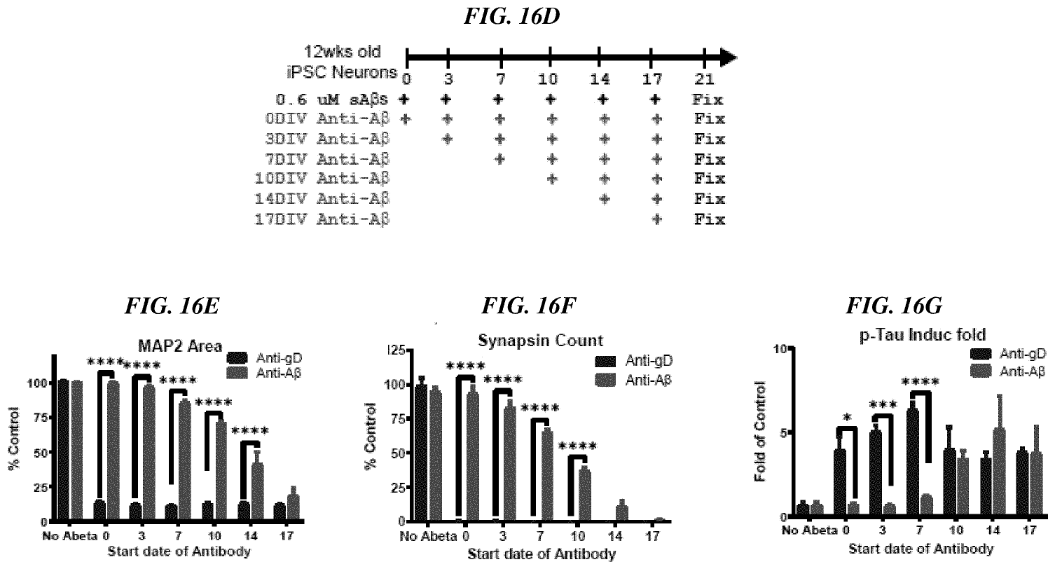

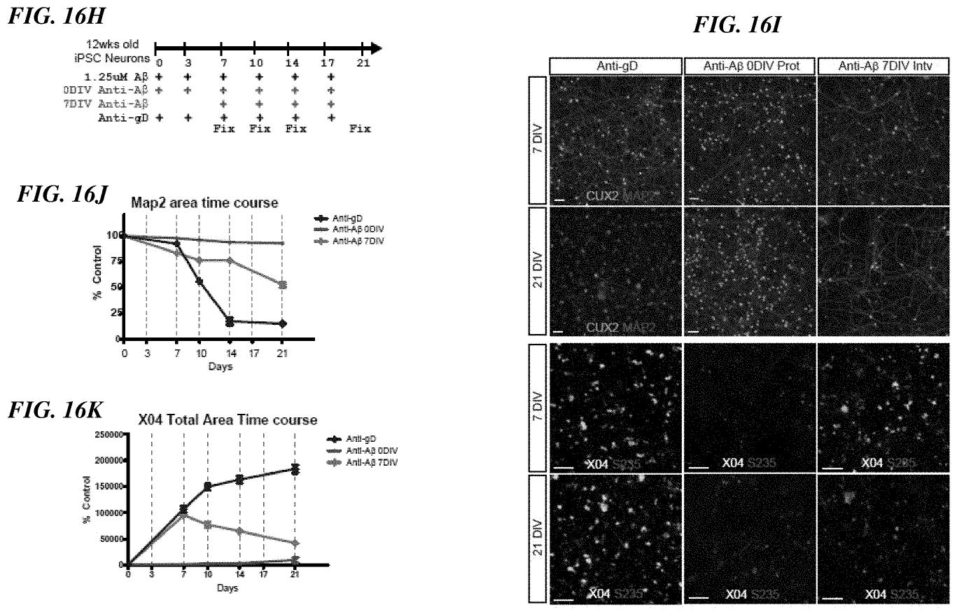

To model the progression of AD and continuous Aβ exposure at a physiological concentration (e.g., a lower elevation of Aβ42 oligomers over an extended time rather than a high single dose of Aβs (5 μM)), repeated doses of Aβ oligomers were added to neuron/astrocyte cultures twice a week after media changes, at several concentrations (0.3 μM-5 μM) over a 21-day time course study. Repeated low dosing of Aβ42 oligomer led to prolonged, increased neuronal toxicity, compared with single exposure (FIG. 16A-16C, solid vs dotted lines). Repeated doses of 0.625 μM were chosen to model AD progression, which took 21 days to cause cell death.

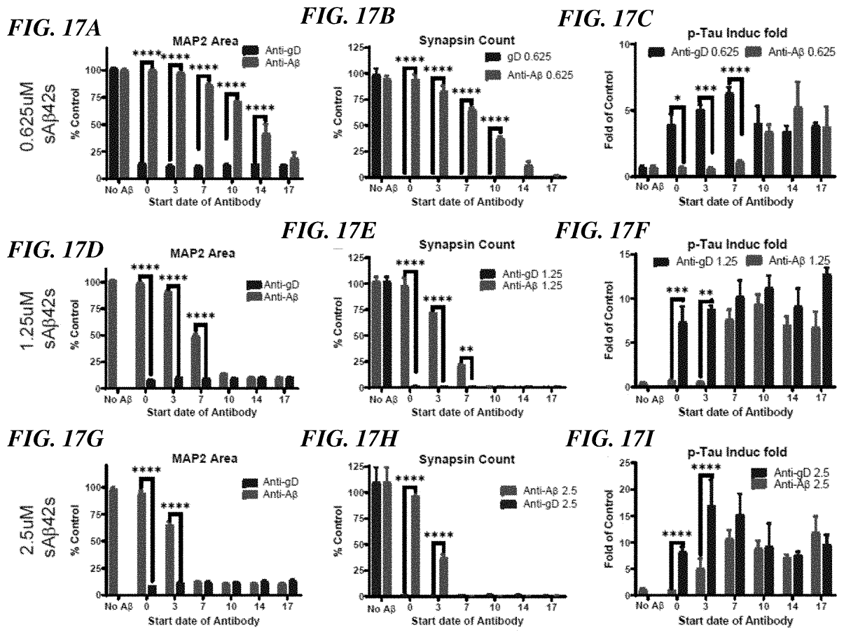

As observed earlier, anti-Aβ antibodies were protective at high concentrations when added at the onset of Aβ exposure (prophylactically). However, in clinical settings, some degree of neuronal damage may have already occurred by the time of therapeutic intervention. To test whether anti-Aβ antibody treatment is effective when AD-induced neurotoxicity has occurred prior to therapeutic treatment, an antibody intervention model was created wherein anti-Aβ treatment began after varying lengths of Aβs exposure (FIG. 16D). Around two-thirds of the way through the disease progression course there is a window where anti-Aβ antibody treatment provides neural protection in dendrites, synapses, and pTau induction (FIGS. 16E-16G). Interestingly, the window of protection against pTau induction is shorter than that of synapsin (7 versus 14 days, respectively), suggesting that anti-Aβ antibody may be most effective prior to pTau induction. Furthermore, the intervention window is shortened when neurodegeneration is sped up by using an increasing amount of Aβs per bi-weekly dose (FIGS. 17A-17I).

Next, a time-course analysis of the MAP2 area as a measure of neuronal health was conducted, with Methoxy-X04 staining for plaque formation, and pTau (S235) as a measure of pTau induction and dystrophic neurites (FIGS. 16H-16K). Anti-Aβ antibodies reduced the progression of neurodegeneration and plaque formation compared with the anti-gD control antibody (FIGS. 16I-16K).

These data demonstrate that the AD model could be modulated to generate a progressive AD disease model with precise temporal control of neurodegeneration speed. When the neuroprotective capabilities of anti-A antibodies were evaluated, it was shown that earlier intervention conferred greater protection.

### Example 8. Anti-Aβ Antibodies Protect Neurons by Keeping Aβ Oligomers in Soluble Supernatant

This example shows that anti-Aβ antibodies confer neuronal protection by restricting Aβ oligomers to the supernatant, where they remain soluble and bound to the antibodies.

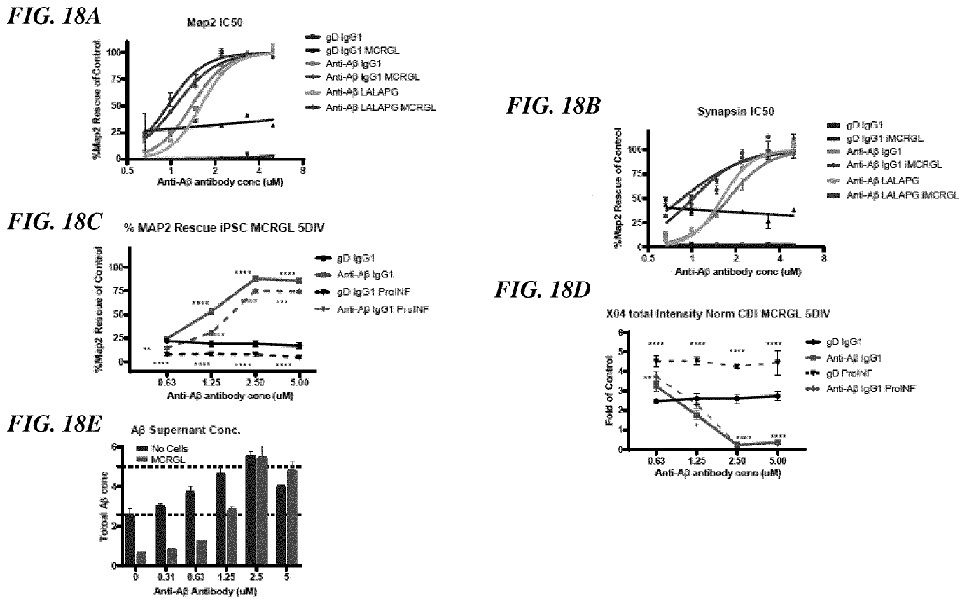

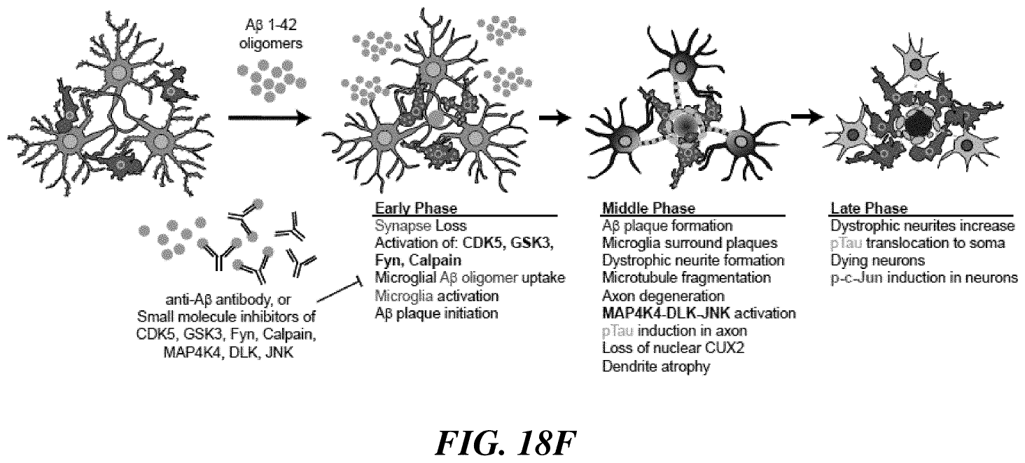

In order to investigate how anti-Aβ antibodies confer neuronal protection in a complete triculture system with microglia, neurons, and astrocytes, the triculture model was treated with Aβ42 oligomers, several anti-Aβ antibodies, and anti-gD antibody controls, with different effector functions: immunoglobulin GI (IgG1; high effector function) and effector-less (LALAPG) antibodies. Antibody IC50 was calculated as a measure of neuronal protection. The anti-gD antibodies were evaluated in the presence or absence of microglia to understand microglial baseline protection, and the antibodies showed ˜25-40% protection of neural synapses and dendrites (FIGS. 18A-18B). Anti-Aβ antibodies showed an increased protection as well as in the presence of microglia, suggesting that microglial neuroprotection and anti-Aβ antibody protection are additive (FIGS. 18A-18B). Comparisons with antibodies with an effector or effector-less function revealed no significant difference, suggesting antibody effector function may not play a role in this model.

To determine whether a neuroinflammatory environment affects antibody protection in the presence of microglia, proinflammatory cytokines IFNγ, IL-1β, and LPS were added to cultures to activate microglia, and neuronal health (MAP2) and plaque formation (Methoxy-X04) were measured. The control anti-gD antibody replicated previous observations (FIGS. 11G-11I), that microglial neuroprotection was lost in a neuroinflammatory state (FIG. 18C). Interestingly, anti-Aβ antibodies were observed to be protective in both contexts, with the IC50 curve shifting to the right, presumably due to the loss of microglial protection (FIG. 18C).

Since the presumed mechanism of action of anti-A antibodies is to bind AD, it was investigated whether Aβs remained solubilized in the supernatant, bound to anti-Aβ antibodies, and/or neutralized from causing toxicity to neurons. The supernatant containing 5 μM Aβs was analyzed with increasing antibody concentrations, and showed that anti-Aβ antibodies increased soluble Aβ in the supernatant while decreasing plate bound Aβ (FIG. 18E). Soluble Aβ present in the supernatant was reduced in the presence of microglia, most likely due to increased plaque formation (FIG. 9C). However, with increasing antibody concentrations, Aβ in the supernatant increased to the original input of 5 μM. This suggests that anti-Aβ antibodies bound and solubilized AD, reducing contact with neurons and microglia, thereby conferring neuronal protection independent of microglia (FIGS. 18D-18E), which is consistent with the observation that anti-Aβ antibodies treatment resulted in decreased plaque formation.

Taken together, the results show the successful generation of an in vivo human iPSC AD model comprised of human neurons, astrocytes and microglia. In this high throughput, triple culture system, the addition of Aβ42 oligomers not only recapitulated the hallmarks of AD, but also developed in a sequential order of events that is similar to human AD disease progression (FIG. 18F).

1. An automated cell culture system for facilitating neuronal differentiation and/or promoting long-term neuronal growth, wherein the automated cell culture system comprises one or more rounds of automated culture media replacements; and wherein the automated cell culture system sustains differentiation, maturation and/or growth of neuronal cells for at least about 30 days.

2. The automated cell culture system of claim 1,
wherein the automated culture media replacement comprises automated culture media aspiration and automated culture media replenishment; and/or
wherein the cell culture system comprises one or more 96-well plates; or one or more 384-well plates.

3. The automated cell culture system of claim 2, wherein the automated culture media aspiration comprises aspiration with a pipet tip, wherein:
the distal end of the pipet tip is at about 1 mm above the bottom surface of the well before, during and/or after the aspiration.

4. The automated cell culture system of claim 2, wherein the automated culture media aspiration comprises aspiration with a pipet tip, wherein:
the pipet tip is at an angle of about 90° to the bottom surface of the well before, during and/or after the aspiration.

5. The automated cell culture system of claim 2, wherein the automated culture media aspiration comprises aspiration with a pipet tip, wherein: the pipet tip has a displacement of no more than 0.1 mm from the center of the well before, during and/or after the aspiration.

6. The automated cell culture system of claim 2, wherein the automated culture media aspiration comprises aspiration with a pipet tip, wherein:
(a) the speed of media aspiration is no more than about 7.5 μl/s; and/or
(b) the start of media aspiration is about 200 ms subsequent to the pipet tip being placed 1 mm above the bottom surface of the well.

7. The automated cell culture system of claim 2, wherein the automated culture media aspiration comprises aspiration with a pipet tip, wherein:
(a) the pipet tip is inserted into the well at a speed of about 5 mm/s prior to aspiration; and/or
(b) the pipet tip is withdrawn from the well at a speed of about 5 mm/s after the aspiration.

8. The automated cell culture system of claim 2, wherein the cell culture system comprises a 384-well plate; further wherein the automated cell culture system comprises automated discarding of a used rack of 384-pipet tips and automated engagement of a new rack of 384-pipet tips subsequent to each round of media aspiration.

9. (canceled)

10. The automated cell culture system of claim 2, wherein the automated culture media replenishment comprises dispensing media with a pipet tip, wherein:
(a) the distal end of the pipet tip is at about 1 mm above the bottom surface of the well before the dispensing; and/or
(b) the pipet tip is withdrawn from the well at a speed of about 1 mm/s during the dispensing.

11. The automated cell culture system of claim 2, wherein the automated culture media replenishment comprises dispensing media with a pipet tip, wherein:
the pipet tip is at an angle of about 90° to the bottom surface of the well before and/or during the dispensing.

12. The automated cell culture system of claim 2, wherein the automated culture media replenishment comprises dispensing media with a pipet tip, wherein:
the pipet tip has a displacement of no more than 0.1 mm from the center of the well before, and/or during the dispensing.

13. The automated cell culture system of claim 2, wherein the cell culture system comprises a 384-well tissue plate; wherein the automated culture media replenishment comprises dispensing media with a pipet tip, wherein:
(a) the pipet tip is displaced to contact a first side of the well 1 mm from the center in a first direction, at a height of about 12.40 mm above the bottom of the well at a speed of about 100 mm/s; and/or
(b) the pipet tip is displaced to contact a second side of the well 1 mm from the center in a second direction, at a height of about 12.40 mm above the bottom of the well at a speed of about 100 mm/s.

14. The automated cell culture system of claim 2, wherein the automated culture media replenishment comprises dispensing media with a pipet tip, wherein:
(a) the speed of media dispensing is no more than about 1.5 μl/s;
(b) the acceleration of media dispensing is about 500 μl/s²;
(c) the deceleration of media dispensing is about 500 μl/s²; and/or
(d) the start of media dispensing is about 200 ms subsequent to the pipet tip being placed 1 mm above the bottom surface of the well.

15. The automated cell culture system of claim 2, wherein the automated culture media replenishment comprises dispensing media with a pipet tip, wherein:
(a) the pipet tip is inserted into the well at a speed of about 5 mm/s prior to dispensing; and/or
(b) the pipet tip is withdrawn from the well at a speed of about 5 mm/s after the dispensing.

16. The automated cell culture system of claim 2, wherein the cell culture system comprises a 384-well plate; further wherein the automated cell culture system comprises automated discarding of a used rack of 384-pipet tips and automated engagement of a new rack of 384-pipet tips subsequent to each round of media dispensing.

17. (canceled)

18. The automated cell culture system of claim 1, wherein the time interval between two rounds of culture media replacements is about 1 to about 10 days.

19. The automated cell culture system of claim 1, wherein the time interval between two rounds of culture media replacements is about 3 or 4 days.

20. The automated cell culture system of claim 1, wherein about 30% to about 80% of culture media is replaced in one or more rounds of culture media replacement.

21.-23. (canceled)

24. A method of generating homogenous and terminally differentiated neurons from pluripotent stem cells, comprising:
(a) generating a pluripotent stem cell- (PSC-) derived neural stem cell (NSC) line expressing NGN2, and ASCL1 under an inducible system;
(b) culturing the NSC line under conditions to induce the expression of NGN2 and ASCL1, in combination with a cell cycle inhibitor for at least about 7 days, thereby generating PSC-derived neurons;
(c) replating the PSC-derived neurons in presence of primary human astrocytes;
(d) differentiating and maturing the PSC-derived neurons for at least about 60 to about 90 days in an automated cell culture system.

25.-32. (canceled)

33. A homogenous population of terminally differentiated neurons derived from pluripotent stem cells, wherein at least 95% of the neurons express: Map2; Synapsin 1 and/or Synapsin 2; and beta-III tubulin.

34. A homogenous population of terminally differentiated neurons derived from pluripotent stem cells, wherein:
(a) at least 95% of the neurons express one or more pre-synaptic markers selected from vGLUT2, Synapsin 1, and Synapsin 2; and/or
(b) at least 95% of the neurons express one or more post-synaptic markers selected from: PSD95, SHANK, PanSHANK, GluR1, GluR2, PanSAPAP, and NR1; and/or
(c) at least 100 postsynaptic endings of a neuron overlap with presynaptic endings of other neurons and/or at least 100 presynaptic endings of the neuron overlap with postsynaptic endings of other neurons.

35.-48. (canceled)

49. A pluripotent stem cell-derived neuronal culture system for use in modeling neurodegenerative diseases,
wherein the neuronal culture system comprises substantially defined culture media and
wherein the neuronal culture system is amenable to modular and tunable inputs of:
one or more disease-associated components and/or
one or more neuroprotective components.

50.-82. (canceled)

83. A method of screening compounds that increase neuroprotection, comprising: contacting the compound with the neuronal culture in the neuronal culture system of claim 49, and quantifying improvements in neuroprotection.

84.-87. (canceled)
